(19) United States
(12) Patent Application Publication
(10) Pub. No.: US 2009/0209621 A1
(43) Pub. Date: Aug. 20, 2009

(54) Compositions and methods for decreasing microrna expression for the treatment of neoplasia

(75) Inventors: Joshua T. Mendell, Baltimore, MD (US); Kathryn A. O'Donnell, Baltimore, MD (US); Karen I. Zeller, Baltimore, MD (US); Erik A. Wentzel, Millersville, MD (US); Chi V. Dang, Baltimore, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, BALTIMORE, MD (US)

(21) Appl. No.: 11/921,532

(22) PCT Filed: Jun. 2, 2006
(86) PCT No.: PCT/US2006/021613

§ 371 Date: Mar. 4, 2009

Related U.S. Application Data
(60) Provisional application No. 60/687,488, filed on Jun. 3, 2005.
(60) Provisional application No. 60/687,756, filed on Jun. 6, 2005.

(51) Int. Cl. A61K 31/7088; C07H 21/04; C12N 15/63; C12N 5/00; C12N 5/02; C12Q 1/68

(52) U.S. Cl. 514/44 A; 536/24.5; 435/320.1; 435/325; 435/375; 435/6

(57) ABSTRACT

The invention generally features compositions and methods that are useful for treating or diagnosing a neoplasia. The invention is based in part on the observation that c-Myc activated expression of a cluster of six miRNAs on human chromosome 13. Accordingly, the invention provides therapeutic compositions and methods for altering the expression of a microRNA of the invention thereby treating a neoplasia, as well as compositions and methods for diagnosing a neoplasia.

### CROSS-REFERENCE TO RELATED APPLICATION

This application claims the benefit of the following U.S. Provisional Application Nos. 60/687,488, which was filed on Jun. 3, 2005, and 60/687,756, which was filed on Jun. 6, 2005, the entire disclosures of which are hereby incorporated in its entirety.

### STATEMENT OF RIGHTS TO INVENTIONS MADE UNDER FEDERALLY SPONSORED RESEARCH

This work was supported by the following grants from the National Institutes of Health, Grant Nos: CA51497 and CA 57341. The government may have certain rights in the invention.

### BACKGROUND OF THE INVENTION

MicroRNAs (miRNAs) are 21-23 nucleotide RNA molecules that regulate the stability or translational efficiency of target mRNAs. miRNAs have diverse functions including the regulation of cellular differentiation, proliferation, and apoptosis. Although strict tissue- and developmental-stage-specific expression is critical for appropriate miRNA function, few mammalian transcription factors that regulate miRNAs have been identified. The proto-oncogene c-MYC encodes a transcription factor that regulates cell proliferation, growth, and apoptosis. Dysregulated expression or function of c-Myc is one of the most common abnormalities in human malignancy.

Cancer causes one in every four US deaths and is the second leading cause of death among Americans. Accordingly, improved compositions and methods for the treatment or prevention of neoplasia are required.

### SUMMARY OF THE INVENTION

As described below, the present invention features compositions and methods for treating or diagnosing a neoplasia in a subject.

In one aspect, the invention provides an inhibitory nucleic acid molecule that is complementary to or corresponds to a microRNA encoded by the miR-17 cluster, where the inhibitory nucleic acid molecule decreases the expression of the microRNA in a cell. In one embodiment, the microRNA is any one or more of mir-17-5p, mir-18a, mir-19a, mir-20a, mir-19b-1, and mir-92-1. In another embodiment, the nucleic acid molecule is an antisense nucleic acid molecule. In yet another embodiment, the microRNA is mir-17-5p or mir-20a. In yet another embodiment, the antisense nucleic acid molecule comprises, consists essentially of, or has at least about 85%, 90%, 95%, or 100% nucleic acid sequence identity to the following nucleic acid sequences:

| | |
|---|---|
| miR-17-5p AS, | 5′-AGUACCUGCACUGUAAGCACUUUG-3′; |
| or | |
| miR-20a AS, | 5′-CUACCUGCACUAUAAGCACUUUA.3′. |

In another aspect, the inhibitory nucleic acid molecule is a double-stranded nucleic acid molecule that corresponds to a microRNA encoded by the miR-17 cluster, wherein the inhibitory nucleic acid molecule decreases the expression of the microRNA in a cell. In one embodiment, the inhibitory nucleic acid molecule is an shRNA or an siRNA. In yet another embodiment, the nucleic acid molecule comprises at least one modification, such as a non-natural internucleotide linkage, modified backbone, or substituted sugar moiety.

In a related aspect, the invention provides an expression vector encoding an inhibitory nucleic acid molecule of any previous aspect. In one embodiment, the vector is a retroviral, adenoviral, adeno-associated viral, or lentiviral vector. In another embodiment, the vector comprises a promoter suitable for expression in a mammalian cell, wherein the promoter is operably linked to the inhibitory nucleic acid molecule.

The invention further provides a cell (e.g., a human neoplastic cell in vivo) comprising the vector of the previous aspect or an inhibitory nucleic acid molecule of any previous aspect.

In another aspect, the invention provides a vector comprising a nucleic acid sequence encoding a reporter gene, wherein the vector further comprises a nucleic acid sequence complementary to a microRNA of the mir-17 cluster (e.g., any one or more of mir-17-5p, mir-18a, mir-19a, mir-20a, mir-19b-1, and mir-92-1), where the complementary sequence is positioned to regulate expression of the reporter gene. In one embodiment, the vector is a sensor vector useful in methods of screening. In another embodiment, the complementary sequence is present in a 3′ untranslated region of the reporter gene.

In a related aspect, the invention provides a cell comprising the above-described vector.

In another aspect, the invention provides a method of decreasing expression of a microRNA of the mir-17 cluster in a cell, the method involving contacting the cell with an effective amount of an inhibitory nucleic acid molecule complementary to at least a portion of a microRNA nucleic acid molecule selected from the group consisting of mir-17-5p, mir-18a, mir-19a, mir-20a, mir-19b-1, and mir-92-1, wherein the inhibitory nucleic acid molecule decreases expression of a microRNA of the mir-17 cluster in the cell. In one embodiment, the inhibitory nucleic acid molecule is an antisense nucleic acid molecule. In another embodiment, the inhibitor nucleic acid molecule decreases expression of mir-17-5p or mir-20a in the cell. In yet another embodiment, the antisense nucleic acid molecule comprises, consists essentially of, or has at least about 85%, 90%, 95%, or 100% nucleic acid sequence identity to the nucleobase sequence of:

| | |
|---|---|
| miR-17-5p AS, | 5′-ACUACCUGCACUGUAAGCACUUUG-3′; |
| or | |
| miR-20a AS, | 5′-CUACCUGCACUAUAAGCACUUUA-3′. |

In another embodiment, the cell is contacted by two or more antisense nucleic acid molecules each of which decreases the expression of a different microRNA.

In yet another aspect, the invention provides a method of treating a subject having a neoplasm, the method comprising administering to the subject an effective amount of an inhibitory nucleic acid molecule complementary to a microRNA of the mir-17 cluster, wherein the inhibitory nucleic acid molecule reduces expression of a microRNA selected from the group consisting of mir-17-5p, mir-18a, mir-19a, mir-20a, mir-19b-1, and mir-92-1 thereby treating the neoplasm. In one embodiment, the microRNA is mir-17-5p or mir-20a.

In yet another aspect, the invention provides a method of treating a subject having a neoplasm (e.g., cancer, such as B-cell lymphoma), the method comprising administering to the subject an effective amount of two inhibitory nucleic acid molecules each of which is complementary to a different microRNA of the mir-17 cluster simultaneously or within 1, 3, 5, 7, 10, 12, 14, or 21 days of each other in amounts sufficient to treat a neoplasm. In one embodiment, the two inhibitory nucleic acid molecules are administered concurrently or within about 14 days of each other in amounts sufficient to inhibit the growth of the neoplasm. In yet another embodiment, one of the inhibitory nucleic acid molecule is complementary to mir-17-5p and one is complementary to mir-20a.

In various embodiments of any of the above aspects, the inhibitory nucleic acid molecule is administered at a dosage of between about 100 to 300 mg/m²/day (e.g., 100, 125, 150, 175, 200, 225, 250, 275, or 300 mg/m²/day).

In another aspect, the invention provides a method of identifying an agent that treats a neoplasm, the method involving contacting a cell that expresses a microRNA of the mir-17 cluster with an agent, and comparing the level of microRNA expression in the cell contacted by the agent with the level of expression in a control cell, wherein an agent that decreases microRNA expression thereby treats a neoplasm. In one embodiment, the decrease in expression is by at least about 5%, 10%, 25%, 50%, 75%, or even by 100%.

In another aspect, the invention provides a method for diagnosing a subject as having or having a propensity to develop a neoplasia (e.g., a cancer, such as B-cell lymphoma), the method involving measuring the level of a marker selected from the group consisting of mir-17-5p, mir-18a, mir-19a, mir-20a, mir-19b-1, mir-92-1, c-Myc, E2F1, and p21 in a biological sample from the subject, and detecting an alteration in the level of the marker in the sample relative to the level in a control sample, wherein detection of an alteration in the marker level indicates the subject has or has a propensity to develop a neoplasia. In one embodiment of the above aspect, the expression of one, two, three, four, five, six, seven, eight, or nine of the markers is measured. In another embodiment, the level of expression is determined in a microarray assay. In yet another embodiment, the method involves measuring the level of mir-17-5p, mir-20a, E2f1 and p21 nucleic acid molecule or polypeptide markers.

In another aspect, the invention features a kit for the diagnosis of a neoplasia in a subject, the kit containing a nucleic acid molecule selected from the group consisting of: mir-17-5p, mir-18a, mir-19a, mir-20a, mir-19b-1, mir-92-1, c-Myc, E2F1, and p21, or a fragment or complement thereof, and written instructions for use of the kit for detection of a neoplasia in a biological sample from the subject.

In another aspect, the invention features a method for identifying an agent that inhibits a neoplasia (e.g., cancer, such as B-cell lymphoma), the method involving contacting a cell (e.g., a HeLa cell or other cell expressing a microRNA of the mir-17 cluster) containing a sensor construct (e.g., a construct of an above aspect) with an agent, wherein the sensor construct comprises a reporter gene linked to a site complementary to a microRNA of the mir-17 cluster; and measuring an alteration in the expression of the reporter gene relative to the expression of the reporter gene from a control vector, wherein the alteration identifies the agent as treating a neoplasia. In one embodiment, the microRNA is miR-17-5p or miR-20a.

In another embodiment, the alteration identifies a compound that downregulates endogenous miR-17-5p or miR-20a expression. In yet another embodiment, the complementary site is present in the 3′ untranslated region (UTR) of the reporter gene (e.g., luciferase).

In yet another aspect, the invention features a method of identifying an agent that inhibits a neoplasia, the method involving contacting a cell (e.g., a cell in vivo or in vitro) that expresses a microRNA of the mir-17 cluster with the agent; and comparing the level of expression of the microRNA in the cell contacted by the candidate compound with the level of expression in a control cell, wherein a decrease in the expression of the microRNA thereby identifies the agent as inhibiting a neoplasia.

In yet another aspect, the invention features a method of identifying an agent that inhibits a neoplasia, the method involving contacting a cell comprising a microRNA of the mir-17 cluster present in an expression vector that includes a reporter construct; and detecting the level of reporter gene expression in the cell contacted with the candidate compound with a control cell not contacted with the candidate compound, wherein a decrease in the level of the reporter gene expression identifies the candidate compound as a candidate compound that inhibits a neoplasia.

In yet another aspect, the invention provides a pharmaceutical composition for treating a neoplasia (e.g., a cancer, such as a B cell lymphoma) in a subject comprising a therapeutically effective amount of an inhibitory nucleic acid molecule that is complementary to at least a fragment of a microRNA of the mir-17 cluster in a pharmaceutically acceptable excipient. In one embodiment, the inhibitory nucleic acid molecule is administered at a dosage of between about 100 to 300 mg/m²/day (e.g., 100, 125, 150, 175, 200, 225, 250, 275, or 300 mg/m²/day). In another embodiment, the inhibitory nucleic acid molecule decreases expression of mir-17-5p or mir-20a, for example, by at least about 5%, 10%, 20%, 30%, 40%, 50%, 60%, 70%, 80%, 90% or more.

In another aspect, the invention provides a pharmaceutical composition for treating a neoplasm (e.g., cancer, such as B cell lymphoma) in a subject (e.g., a human patient) containing an effective amount of an inhibitory nucleic acid molecule that corresponds to or is complementary to at least a fragment of mir-17-5p or mir-20a in a pharmaceutically acceptable excipient. In one embodiment, the composition comprises an inhibitory nucleic acid molecule complementary to mir-17-5p and mir-20a.

In another aspect, the invention provides a packaged pharmaceutical containing an effective amount of an inhibitory nucleic acid molecule complementary to at least a fragment of a microRNA of the mir-17 cluster, and that decreases expression of the microRNA in a cell, and instructions for use in treating a subject having a neoplasm.

In yet another aspect, the invention provides a packaged pharmaceutical comprising an effective amount of an inhibitory nucleic acid molecule corresponding to or complementary to at least a fragment of a microRNA of the mir-17 cluster, and that decreases expression of the microRNA in a cell, and instructions for use in treating or preventing neoplasia in a subject.

In various embodiments of the above aspects, the method further involves obtaining the inhibitory nucleic acid molecule.

In yet another aspect, the invention provides a nucleic acid probe that binds a microRNA sequence and that has a nucleic acid sequence selected from those listed in Table 1 or that is complementary to a microRNA sequence encoded by the mir-17 cluster.

In yet another aspect, the invention provides a nucleic acid probe that hybridizes with a microRNA sequence and that has at least about 85% identity to, comprises, or consists essentially of a nucleic acid sequence selected from the group consisting of:

miR-17 cluster probe:

| | |
|---|---|
| sense | 5′-ACATGGACTAAATTGCCTTTAAATG-3′, |
| antisense | 5′-AATCTTCAGTTTTACAAGGTGATG-3; |
| and | |

miR-106a cluster probe:

| | |
|---|---|
| sense | 5′-CATCCTGGGTTTTACATGCTCC-3′, |
| antisense | 5′-CAAAATTTTAAGTCTTCCAGGAGC-3′. |

In various embodiments of any of the above aspects, the inhibitory nucleic acid molecule is an antisense molecule, an shRNA molecule, or an siRNA molecule that corresponds to or is complementary to a microRNA encoded by the mir-17 cluster. In other embodiments of the above aspects, such inhibitory nucleic acids molecules are used to decrease the expression of a microRNA encoded by the mir-17 cluster in a cell, such as the cell of a subject (e.g., a human patient) for the treatment of a neoplasm (e.g., a cancer, such as lung cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer). In still other embodiments of the above aspects, an inhibitory nucleic acid molecule complementary to mir-17-5p is used to treat a subject having breast, colon, lung, pancreas, or prostate cancer (e.g., a solid tumor affecting these organs). In other embodiments of the above aspects, an inhibitory nucleic acid molecule complementary to mir-20a is used to treat colon, pancreas, or prostate cancer (e.g., a solid tumor affecting one of those organs). In other embodiments of the above aspects, the antisense nucleic acid molecule comprises, consists essentially of, or has at least about 85% sequence identity to the following nucleic acid sequences:

| | |
|---|---|
| miR-17-5p AS, | 5′-ACUACCUGCACUGUAAGCACUUUG-3′; |
| or | |
| miR-20a AS, | 5′-CUACCUGCACUAUAAGCACUUUA-3′. |

In other embodiment of any of the above aspects, the expression of one, two, three, four, five, six, seven, eight, or nine of the markers is measured in a biological sample obtained from a subject for the diagnosis of a neoplasia. In another embodiment, the cell is contacted by two or more antisense nucleic acid molecules each of which decreases the expression of a microRNA. In other embodiments of the above aspects, the expression of one, two, three, four, five, six, seven, eight, or nine of the markers is measured. In yet other embodiments, the level of expression is determined in a microarray assay. In still other embodiments, the method involves measuring the level of mir-17-5p, mir-20a, E2f1 and p21 nucleic acid molecule or polypeptide markers.

Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide, polynucleotide, or fragment, or analog thereof, small molecule, or other biologically active molecule.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “antisense molecule” is meant a non-enzymatic nucleic acid molecule or analog or variant thereof that binds to a target nucleic acid molecule sequence by means of complementary base pairing, such as an RNA-RNA or RNA-DNA interactions and alters the expression of the target sequence. Typically, antisense molecules are complementary to a target sequence along a single contiguous sequence of the antisense molecule. In certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of a target sequence.

The phrase “in combination with” is intended to refer to all forms of administration that provide an inhibitory nucleic acid molecule together with a second agent, such as a second inhibitory nucleic acid molecule or a chemotherapeutic agent, where the two are administered concurrently or sequentially in any order.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “control” is meant a standard or reference condition.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more.

By “an effective amount” is meant the amount of an agent required to ameliorate the symptoms of a disease relative to an untreated patient. The effective amount of active agent(s) used to practice the present invention for therapeutic treatment of a neoplasia varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibits a neoplasia” is meant decreases the propensity of a cell to develop into a neoplasia or slows, decreases, or stabilizes the growth or proliferation of a neoplasia.

By “inhibitory nucleic acid molecule” is meant a single stranded or double-stranded RNA, siRNA (short interfering RNA), shRNA (short hairpin RNA), or antisense RNA, or a portion thereof, or an analog or mimetic thereof, that when administered to a mammalian cell results in a decrease (e.g., by 10%, 25%, 50%, 75%, or even 90-100%) in the expression of a target sequence. Typically, a nucleic acid inhibitor comprises or corresponds to at least a portion of a target nucleic acid molecule, or an ortholog thereof, or comprises at least a portion of the complementary strand of a target nucleic acid molecule.

By “marker” is meant any protein or polynucleotide having an alteration in expression level or activity that is associated with a disease or disorder.

The term “microarray” is meant to include a collection of nucleic acid molecules or polypeptides from one or more organisms arranged on a solid support (for example, a chip, plate, or bead).

By “miR-17 cluster” is meant the cluster of microRNAs located on chromosome 13 that encodes miR5-17-5p, 18a, 19a, 20a, 19-b1, and 92-1. The sequence of the primary transcript containing all the microRNAs present in the cluster is provided at GenBank Accession No. AB176708.

By “mir-17-5p” is meant a microRNA comprising or having at least 85% identity to the nucleic acid sequence provided at Genbank Accession No. AF480529.

By “mir-18a” is meant a microRNA comprising or having at least 85% identity to the nucleic acid sequence provided at GenBank Accession No. AJ421736. By “mir-119a” is meant a microRNA comprising or having at least 85% identity to the nucleic acid sequence provided at GenBank Accession No. AJ421737.

By “mir-20a” is meant a microRNA comprising or having at least 85% identity to the nucleic acid sequence provided at Genbank Accession No. AJ421738.

By “mir-19b-1” is meant a microRNA comprising or having at least 85% identity to the nucleic acid sequence provided at Genbank Accession No. AJ421739.

By “mir-92-1 is meant a microRNA comprising or having at least 85% identity to the nucleic acid sequence provided at Genbank Accession No. AF480530.

By “modification” is meant any biochemical or other synthetic alteration of a nucleotide, amino acid, or other agent relative to a naturally occurring reference agent.

By “neoplasia” is meant any disease that is caused by or results in inappropriately high levels of cell division, inappropriately low levels of apoptosis, or both. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia Vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant microRNA molecule described herein).

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “reference” is meant a standard or control condition.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

The term “siRNA” refers to small interfering RNA; a siRNA is a double stranded RNA that “corresponds” to or matches a reference or target gene sequence. This matching need not be perfect so long as each strand of the siRNA is capable of binding to at least a portion of the target sequence. SiRNA can be used to inhibit gene expression, see for example Bass, 2001, Nature, 411, 428 429; Elbashir et al., 2001, Nature, 411, 494 498; and

Zamore et al., Cell 101:25-33 (2000).

The term “subject” is intended to include vertebrates, preferably a mammal. Mammals include, but are not limited to, humans.

The term “pharmaceutically-acceptable excipient” as used herein means one or more compatible solid or liquid filler, diluents or encapsulating substances that are suitable for administration into a human.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and still more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “targets” is meant alters the biological activity of a target polypeptide or nucleic acid molecule.

By “transformed cell” is meant a cell into which (or into an ancestor of which) has been introduced, by means of recombinant DNA techniques, a polynucleotide molecule encoding (as used herein) a protein of the invention.

By “vector” is meant a nucleic acid molecule, for example, a plasmid, cosmid, or bacteriophage, that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

### BRIEF DESCRIPTION OF THE DRAWINGS

FIGS. 1A and 1B show microRNA expression profiling of P493-6 cells with high and low c-Myc expression. FIG. 1A is a Western blot analysis of c-Myc in untreated cells or in cells treated with tetracycline (tet). Blots were stripped and reprobed for α-tubulin to demonstrate equal loading. FIG. 1B shows microRNA expression arrays hybridized with RNA from tet-treated (+tet) or untreated (−tet) cells. Magnified panels show miRNAs that were consistently upregulated in the high c-Myc state. A probe complementary to threonine tRNA (tRNA^{thr}) served as a control for equal hybridization.

FIGS. 2A-2D show that c-Myc induces expression of the miR-17 cluster. FIG. 2A provides a schematic representation of the miR-17, miR-106a, and miR-106b clusters. miR-18b and miR-20b are predicted based on homology to miR-18a and miR-20a, respectively (Tanzer et al., J Mol Biol 339, 327-35 (2004)). FIG. 2B shows a Northern blot analysis of mRNAs in P493-6 cells. Duplicate samples are shown. miR-30 served as a loading control. Blots were also probed for miR-16 and miR-29 as loading controls and similar results were obtained (data not shown). FIG. 2C shows a Northern blot analysis of total RNA from P493-6 cells with a probe specific for the miR-17 cluster. 7SK RNA served as a loading control. FIG. 2D shows a Northern blot analysis of miRNAs in wild-type rat fibroblasts (+/+), rat fibroblasts with a homozygous deletion of c-Myc (−/−), or knockout fibroblasts reconstituted with wild-type c-Myc [−/−(c-Myc)]. Quantitation is shown on the right.

FIGS. 3A-3E shows that c-Myc binds directly to the miR-17 cluster genomic locus. FIG. 3A provides a schematic representation of the genomic interval encompassing the miR-17 cluster. Putative c-Myc binding sites are indicated (CACGTG or CATGTG); those in gray are conserved between human and mouse. The location and structure of the C13orf25 transcript is indicated. Real-time PCR amplicons are represented by numbered lines. FIG. 3B is a graph showing a real-time PCR analysis of c-Myc chromatin immunoprecipitates. Amplification of a validated c-Myc binding site in intron 1 of the B23 gene served as a positive control (Zeller et al. J Biol Chem 276, 48285-91 (2001)). FIG. 3C shows a Western blot analysis of c-Myc protein levels following serum stimulation of primary human fibroblasts. FIGS. 3D and 3E are graphs showing a real-time PCR analysis miR-17 cluster expression (FIG. 3D) and c-Myc chromatin immunoprecipitates (FIG. 3E) in serum-stimulated fibroblasts. Error bars for all panels represent standard deviations derived from at least three independent measurements.

FIGS. 4A-4H show that miR-17-5p and miR-20a regulate E2F1 translational yield.

FIG. 4A is a graph that quantitates the inhibition of miR-17-5p and miR-20a by 2′-O-methyl-oligoribonucleotides. Sensor or control luciferase constructs were transfected into HeLa cells alone (mock) or with the following oligonucleotides: 20 or 40 pmol of scrambled (scramble 20 or scramble 40), 20 pmol of miR-17-5p or miR-20a antisense individually (miR-17-5p AS, miR-20a AS) or pooled (miR-17-5p+20a AS). The ratio of normalized sensor to control luciferase activity is shown. Error bars represent standard deviations. FIG. 4B shows a Western blot and FIG. 4C shows a northern blot analysis of E2F1 in antisense-treated HeLa cells. FIG. 4D shows a Northern blot analysis of miR-20 in transfected HeLa cells. FIG. 4E shows a Western blot and FIG. 4F shows a northern blot analysis of E2F1 in transfected HeLa cells. FIG. 4G shows a Northern blot and FIG. 4H shows a western blot analysis of E2F1 in P493-6 cells. Fold changes shown are mean values derived from three experiments.

FIGS. 5A-5C show that E2F1 mRNA is directly regulated by miR-17-5p and miR-20a. FIG. 5A is a schematic representation of the E2F1 transcript. Predicted miR-17-5p and miR-20a binding sites are depicted (site 1 and site 2). The numbers (+387-393) and (+980-986) represent the nucleotides (numbered relative to the position of the E2F1 termination codon) that are predicted to base-pair with nucleotides 2-7 of the miRNA (the miRNA “seed sequence”) (Lewis et al., Cell 115: 787-98, 2003). FIG. 5B shows the sequences of the predicted miRNA binding sites in five mammalian genomes. Highly conserved nucleotides are shown in gray. The mutations introduced into luciferase reporter constructs are shown below the alignments in gray font. FIG. 5C is a box plot showing the normalized luciferase activity resulting from transfection of wild-type or mutant reporter constructs. As a positive control, cells were transfected with wild-type or mutant constructs containing a portion of the PTEN 3′ UTR which has previously been shown to be regulated by miR-19a (Bartel et al., Cell 116, 281-97 (2004)). Each box represents the range of activities observed (n=10-12). The ends of the boxes represent the 25th and 75th percentiles, the bars indicate the 10th and 90th percentiles, and a line shows the median. The number associated with the mutant boxes shows the median fold increase in activity compared to the wild-type construct.

FIGS. 6A-6C show the delayed accumulation of E2F1, but not c-Myc. FIG. 6A shows a Northern blot analysis of E2F1 and c-Myc mRNA during a serum stimulation time-course. FIG. 6B shows a Western blot analysis of E2F1 protein during a serum stimulation time-course. FIG. 6C shows the quantitation of blots shown in panels 6A and 6B. c-Myc protein levels were derived from blot shown in FIG. 3C.

FIG. 7 shows predicted miRNA binding sites in the p213′ UTR The numbers above the sites (+474 and +1154) indicate the position of the nucleotide that is predicted to base-pair with the first nucleotide of miR-20a (numbered relative to the termination codon of p21). Note that the sequence of miR-17-5p is nearly identical to miR-20a and thus this miRNA is also predicted to bind to these sites

FIG. 8A-8C overexpression or inhibition of the miR-17 cluster leads to altered expression of p21. FIG. 8A is a Northern blot demonstrating increased miR-20 expression in TRE-miR17 HeLa cells upon removal of tetracycline (tet). FIG. 8B shows a Western blot demonstrating downregulation of p21 when the miR-17 cluster is induced by tet withdrawal. FIG. 8C is a Western blot demonstrating increased expression of p21 when miR-17-5p or miR-20a are inhibited with 2′-O-methyl antisense oligos. Cells were mock transfected, transfected with 20 or 40 pmol of scrambled oligo (scramble 20, scramble 40), or transfected with 20 pmol of miR-17-5p or miR-20a antisense, either individually (miR-17-5p AS or miR-20a AS) or pooled (miR-17-5p+20a AS).

FIGS. 9A-9B show that the p21 transcript is directly regulated by mir-17-5p and mir-20a. FIG. 9A shows the nucleic acid sequences at site 1 (MUT1) and site 2 (MUT2) of the p21 reporter construct with mutations at those sites shown in gray. FIG. 9B is a box plot showing the normalized luciferase activity resulting from transfection of wild-type or mutant reporter constructs. As a positive control, cells were transfected with wild-type or mutant constructs containing a portion of the PTEN 3′ UTR, which has previously been shown to be regulated by miR-19a. Each box represents the range of activities observed (n=10-12). The ends of the boxes represent the 25th and 75th percentiles, the bars indicate the 10th and 90th percentiles, and a line shows the median. The number associated with the mutant boxes shows the median fold increase in activity compared to the wild-type construct.

### DETAILED DESCRIPTION OF THE INVENTION

The invention generally features compositions and methods that are useful for treating or diagnosing a neoplasia. The invention is based in part on the observation that c-Myc activated expression of a cluster of six miRNAs on human chromosome 13. Chromatin immunoprecipation demonstrated that c-Myc binds directly to this locus. The transcription factor E2F1 is an additional target of c-Myc that promoted cell cycle progression. Evidence that expression of E2F1 and p21 is regulated by two miRNAs in this cluster, miR-17-5p and miR-20a, is also presented. Accordingly, the invention provides compositions and methods for altering the expression of a microRNA of the invention thereby treating a neoplasia.

MicroRNAs of the mir-17 Cluster

MicroRNAs (miRNAs) are 21-23 nucleotide RNA molecules that regulate the stability or translational efficiency of target mRNAs. miRNAs have diverse functions including the regulation of cellular differentiation, proliferation, and apoptosis (Ambros, Nature 431, 350-5 (2004)). Although strict tissue- and developmental-stage-specific expression is critical for appropriate miRNA function, few mammalian transcription factors that regulate miRNAs have been identified. The proto-oncogene c-MYC encodes a transcription factor that regulates cell proliferation, growth, and apoptosis (Levens, Proc Natl Acad Sci USA 99, 5757-9 (2002). Dysregulated expression or function of c-Myc is one of the most common abnormalities in human malignancy (Cole et al., Oncogene 18, 2916-24 (1999)). As reported herein, c-Myc activated expression of a cluster of six miRNAs on human chromosome 13. Chromatin immunoprecipation demonstrated that c-Myc bound directly to this locus. The transcription factor E2F1 is an additional target of c-Myc that promotes cell cycle progression (Bracken et al., Trends Biochem Sci 29, 409-17 (2004); Leone et al., Nature 387, 422-6 (1997); and Fernandez, et al. Genes Dev 17, 1115-29 (2003)). Further evidence reported herein indicated that E2F1 is regulated by two miRNAs in this cluster, miR-17-5p and miR-20a. These findings expand the known classes of transcripts within the c-Myc target gene network and reveal a novel mechanism through which c-Myc simultaneously activated and limited expression of a target gene, allowing a tightly-controlled proliferative signal.

### Inhibitory Nucleic Acid Molecules

Given that c-Myc activation of microRNAs of the mir-17 cluster is associated with cancer, the invention provides compositions that inhibit the expression of these microRNAs as well as methods of using such compositions for the treatment of cancer. In one embodiment, the invention provides inhibitory nucleic acid molecules, such as antisense nucleic acid molecules, that decrease the expression of at least one microRNA of the mir-17 cluster. Inhibitory nucleic acid molecules are essentially nucleobase oligomers that may be employed to decrease the expression of a target nucleic acid sequence, such as a nucleic acid sequence that encodes a microRNA of the mir-17 cluster. The inhibitory nucleic acid molecules provided by the invention include any nucleic acid molecule sufficient to decrease the expression of a nucleic acid molecule of the mir-17 cluster by at least 5-10%, desirably by at least 25%-50%, or even by as much as 75%-100%. Each of the nucleic acid sequences provided herein may be used, for example, in the discovery and development of therapeutic antisense nucleic acid molecules to decrease the expression of a microRNA encoded by the mir-17 cluster (e.g., mir-17-5p or mir-20a). If desired, antisense nucleic acid molecules that target one or more microRNAs of the mir-17 cluster are administered in combination, such that the coordinated reduction in the expression of two or more microRNAs encoded by the mir-17 cluster is achieved.

The invention is not limited to antisense nucleic acid molecules but encompasses virtually any single-stranded or double-stranded nucleic acid molecule that decreases expression of a microRNA within the mir-17 cluster. The invention further provides catalytic RNA molecules or ribozymes. Such catalytic RNA molecules can be used to inhibit expression of a microRNA nucleic acid molecule in vivo.

The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Nucleic Acids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

In another approach, the inhibitory nucleic acid molecule is a double-stranded nucleic acid molecule used for RNA interference (RNAi)-mediated knock-down of the expression of a microRNA. siRNAs are also useful for the inhibition of microRNAs. See, for example, Nakamoto et al., Hum Mol Genet, 2005. Desirably, the siRNA is designed such that it provides for the cleavage of a target microRNA of the invention. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to a microRNA of the mir-17 cluster comprises at least a fragment of the double-stranded gene, such that each strand of the double-stranded inhibitory nucleic acid molecule is capable of binding to the complementary strand of the target gene. The inhibitory nucleic acid molecule need not have perfect correspondence or need not be perfectly complementary to the reference sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

Inhibitory nucleic acid molecules of the invention also include double stranded nucleic acid “decoys.” Decoy molecules contain a binding site for a transcription factor that is responsible for the deregulated transcription of a gene of interest. The present invention provides decoys that competitively block binding to a regulatory element in a target gene (e.g., mir-17 cluster). The competitive inhibition of c-Myc binding by the decoy results in the indirect inhibition of transcription of a target microRNA of the mir-17 cluster. An overview of decoy technology is provided by Suda et al., Endocr. Rev., 1999, 20, 345-357; S. Yla-Hertttuala and J. F. Martin, The Lancet 355, 213-222, 2000). In one therapeutic method, short double-stranded DNA decoy molecules are introduced into cells (e.g., neoplastic cells) of a subject. The decoys are provided in a form that facilitates their entry into target cells of the subject. Having entered a cell, the decoy specifically binds an endogenous transcription factor, thereby competitively inhibiting the transcription factor from binding to an endogenous gene. The decoys are administered in amounts and under conditions whereby binding of the endogenous transcription factor to the endogenous gene is effectively competitively inhibited without significant host toxicity. Depending on the transcription factor, the methods can effect up- or down-regulation of gene expression. The subject compositions comprise the decoy molecules in a context that provides for pharmacokinetics sufficient for effective therapeutic use.

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day. Desirably, a human patient having a neoplasia receives a dosage between about 50 and 300 mg/m²/day (e.g., 50, 75, 100, 125, 150, 175, 200, 250, 275, and 300).

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with a nucleic acid molecule of the mir-17 cluster. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In other embodiments, a single stranded modified nucleic acid molecule (e.g., a nucleic acid molecule comprising a phosphorothioate backbone and 2′-O-Me sugar modifications is conjugated to cholesterol. Such conjugated oligomers are known as “antagomirs.” Methods for silencing microRNAs in vivo with antagomirs are described, for example, in Krutzfeldt et al., Nature 438: 685-689.

### Mir-17 Cluster Polynucleotides

In general, the invention includes any nucleic acid sequence encoding a microRNA of the mir-17 cluster as well as nucleic acid molecules containing at least one strand that hybridizes with a nucleic acid sequence of the mir-17 cluster (e.g., an inhibitory nucleic acid molecule, such as an antisense molecule, a dsRNA, siRNA, or shRNA). The inhibitory 10 nucleic acid molecules of the invention can be between 8 and 45 nucleotides in length. In some embodiments, the inhibitory nucleic acid molecules of the invention comprises 8, 10, 15, 16, 17, 18, 19, 20, 21, 22, 23, 24, 25, 26, 27, 28, 29, 30, 31, 32, 33, 34, 35, 45, or complementary nucleotide residues. In yet other embodiments, the antisense molecules are 60%, 65%, 70%, 75%, 80%, 85%, 90%, 95%, 96%, 97%, 98%, 99% complementary to the target sequence. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

### Delivery of Nucleobase Oligomers

Naked oligonucleotides are capable of entering tumor cells and inhibiting the expression of a microRNA of the mir-17 cluster (e.g., mir-17-5p or mir-20a). Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Polynucleotide Therapy

Polynucleotide therapy featuring a polynucleotide encoding an inhibitory nucleic acid molecule or analog thereof that targets a microRNA of the mir-17 cluster is another therapeutic approach for treating a neoplasia in a subject. Expression vectors encoding inhibitory nucleic acid molecules can be delivered to cells of a subject having a neoplasia. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, antiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an inhibitory nucleic acid molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of an inhibitory nucleic acid molecule therapeutic to a cell of a patient diagnosed as having a neoplasia. For example, an inhibitory nucleic acid molecule that targets a microRNA of the mir-17 cluster can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the inhibitory nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

Inhibitory nucleic acid molecule expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Pharmaceutical Compositions

As reported herein, an increase in the expression of microRNAs of the mir-17 cluster is associated with cancer. Accordingly, the invention provides therapeutic compositions that decrease the expression of a microRNAs of the mir-17 cluster to treat neoplasia. In one embodiment, the present invention provides a pharmaceutical composition comprising an inhibitory nucleic acid molecule (e.g., an antisense, siRNA, or shRNA polynucleotide) that decreases the expression of one or more nucleic acid molecules encoded by the mir-17 cluster (e.g., mir-17-5p or mir-20a). If desired, the inhibitory nucleic acid molecule is administered in combination with a chemotherapeutic agent. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

An inhibitory nucleic acid molecule of the invention, or other negative regulator of a microRNA encoded by the mir-17 cluster (e.g., mir-17-5p or mir-20a) may be administered within a pharmaceutically-acceptable diluent, carrier, or excipient, in unit dosage form. Conventional pharmaceutical practice may be employed to provide suitable formulations or compositions to administer the compounds to patients suffering from a neoplasia (e.g., cancer). Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracistemal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase oligomer of the invention is likely to depend on such variables as the type and extent of the disorder, the overall health status of the particular patient, the formulation of the compound excipients, and its route of administration.

With respect to a subject having a neoplastic disease or disorder, an effective amount is sufficient to stabilize, slow, or reduce the proliferation of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an antisense targeting the mir-17 cluster (e.g., mir-17-5p or mir-20a).

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. As described above, if desired, treatment with an inhibitory nucleic acid molecule of the invention may be combined with therapies for the treatment of proliferative disease (e.g., radiotherapy, surgery, or chemotherapy). For any of the methods of application described above, an inhibitory nucleic acid molecule of the invention is desirably administered intravenously or is applied to the site of neoplasia (e.g., by injection).

### Diagnostics

As described in more detail below, the present invention has identified increases in the expression of microRNAs of the mir-17 cluster and c-Myc, and corresponding decreases in the expression of E2F1 or p21 expression that are associated with cancer. Thus, alterations in the expression level of one or more (e.g., 1, 2, 3, 4, 5, 6, 7, 8, or 9) of the following markers is used to diagnose a neoplasia: mir-17-5p, mir-18a, mir-19a, mir-20a, mir-19b-1, mir-92-1, c-Myc, E2F1, and p21. If desired, alterations in the expression of all of these markers is used to diagnose or characterize a neoplasia.

In one embodiment, a subject is diagnosed as having or having a propensity to develop a neoplasia, the method comprising measuring markers in a biological sample from a patient, and detecting an alteration in the expression of test marker molecules relative to the sequence or expression of a reference molecule. The markers typically include a microRNA of the mir-17 cluster together with c-Myc. While the following approaches describe diagnostic methods featuring a microRNA of the mir-17 cluster, the skilled artisan will appreciate that any one or more of the markers set forth above is useful in such diagnostic methods.

Increased expression of a microRNA of the mir-17 cluster is correlated with neoplasia. Accordingly, the invention provides compositions and methods for identifying a neoplasia in a subject. The present invention provides a number of diagnostic assays that are useful for the identification or characterization of a neoplasia. Alterations in gene expression are detected using methods known to the skilled artisan and described herein. Such information can be used to diagnose a neoplasia.

In one approach, diagnostic methods of the invention are used to assay the expression of a microRNA of the mir-17 cluster in a biological sample relative to a reference (e.g., the level of microRNA of the mir-17 cluster present in a corresponding control tissue). In one embodiment, the level of a microRNA of the mir-17 cluster is detected using a nucleic acid probe that specifically binds a microRNA of the mir-17 cluster. Exemplary nucleic acid probes that specifically bind a microRNA of the mir-17 cluster are described herein. By “nucleic acid probe” is meant any nucleic acid molecule, or fragment thereof, that binds a microRNA encoded by the mir-17 cluster. Such nucleic acid probes are useful for the diagnosis of a neoplasia.

In one approach, quantitative PCR methods are used to identify an increase in the expression of a microRNA encoded by the mir-17 cluster. In another approach, PCR methods are used to identify an alteration in the sequence of a microRNA encoded by the mir-17 cluster. The invention provides probes that are capable of detecting a microRNA encoded by the mir-17 cluster. Such probes may be used to hybridize to a nucleic acid sequence derived from a patient having a neoplasia. The specificity of the probe determines whether the probe hybridizes to a naturally occurring sequence, allelic variants, or other related sequences. Hybridization techniques may be used to identify mutations indicative of a neoplasia or may be used to monitor expression levels of these genes (for example, by Northern analysis (Ausubel et al., supra).

In general, the measurement of a nucleic acid molecule in a subject sample is compared with a diagnostic amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant increase or decrease (e.g., at least about 10%, 15%, 30%, 50%, 60%, 75%, 80%, or 90%) in the level of test nucleic acid molecule or polypeptide in the subject sample relative to a reference may be used to diagnose a neoplasia. Test molecules include any one or more of mir-17-5p, mir-18a, mir-19a, mir-20a, mir-19b-1, mir-92-1, c-Myc, E2F1, and p21. In one embodiment, the reference is the level of test polypeptide or nucleic acid molecule present in a control sample obtained from a patient that does not have a neoplasia. In another embodiment, the reference is a baseline level of test molecule present in a biologic sample derived from a patient prior to, during, or after treatment for a neoplasia. In yet another embodiment, the reference can be a standardized curve.

### Types of Biological Samples

The level of markers in a biological sample from a patient having or at risk for developing a neoplasia can be measured, and an alteration in the expression of test marker molecule relative to the sequence or expression of a reference molecule, can be determined in different types of biologic samples. Test markers include any one or all of the following: mir-17-5p, mir-18a, mir-19a, mir-20a, mir-19b-1, mir-92-1, c-Myc, E2F1, and p21. The biological samples are generally derived from a patient, preferably as a bodily fluid (such as blood, cerebrospinal fluid, phlegm, saliva, or urine) or tissue sample (e.g. a tissue sample obtained by biopsy).

### Kits

The invention provides kits for the diagnosis or monitoring of a neoplasia, such as a neoplasia. In one embodiment, the kit detects an alteration in the expression of a Marker (e.g., mir-17-5p, mir-18a, mir-19a, mir-20a, mir-19b-1, mir-92-1, c-Myc, E2F1, and p21) nucleic acid molecule relative to a reference level of expression. In another embodiment, the kit detects an alteration in the sequence of a mir-17 cluster nucleic acid molecule (e.g., a micrRNA of the cluster, such as mir-17-5p, mir-18a, mir-19a, mir-20a, mir-19b-1, mir-92-1) derived from a subject relative to a reference sequence. In related embodiments, the kit includes reagents for monitoring the expression of a mir-17 cluster nucleic acid molecule, such as primers or probes that hybridize to a mir-17 cluster nucleic acid molecule.

Optionally, the kit includes directions for monitoring the nucleic acid molecule levels of a Marker in a biological sample derived from a subject. In other embodiments, the kit comprises a sterile container which contains the primer, probe, antibody, or other detection regents; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a neoplasia can be monitored using the methods and compositions of the invention. In one embodiment, the disease state of a patient can be monitored using the methods and compositions of the invention. Such monitoring may be useful, for example, in assessing the efficacy of a particular drug in a patient. Therapeutics that alter the expression of any one or more of the Markers of the invention (e.g., mir-17-5p, mir-18a, mir-19a, mir-20a, mir-19b-1, mir-92-1, c-Myc, E2F1, and p21) are taken as particularly useful in the invention.

### Screening Assays

One embodiment of the invention encompasses a method of identifying an agent that inhibits the expression or activity of a microRNA of the mir-17 cluster. Accordingly, compounds that modulate the expression or activity of a mir-17 cluster nucleic acid molecule, variant, or portion thereof are useful in the methods of the invention for the treatment or prevention of a neoplasm (e.g., breast, colon, lymph, ovary, stomach, thyroid, testis, and uterine cancer). The method of the invention may measure a decrease in transcription of one or more microRNAs of the invention or an alteration in the transcription or translation of the target of such a microRNA (e.g., p21, or E2F1). Any number of methods are available for carrying out screening assays to identify such compounds. In one approach, the method comprises contacting a cell that expresses a microRNA with an agent and comparing the level of microRNA expression in the cell contacted by the agent with the level of expression in a control cell, wherein an agent that decreases the expression of a mir-17 cluster microRNA expression thereby inhibits a neoplasia. In another approach, candidate compounds are identified that specifically bind to and alter the activity of a microRNA of the invention. Methods of assaying such biological activities are known in the art and are described herein. The efficacy of such a candidate compound is dependent upon its ability to interact with a mir-17 cluster microRNA. Such an interaction can be readily assayed using any number of standard binding techniques and functional assays (e.g., those described in Ausubel et al., supra).

Potential agonists and antagonists of a mir-17 cluster microRNA include organic molecules, peptides, peptide mimetics, polypeptides, nucleic acid molecules (e.g., double-stranded RNAs, siRNAs, antisense polynucleotides), and antibodies that bind to a nucleic acid sequence of the invention and thereby inhibit or extinguish its activity. Potential antagonists also include small molecules that bind to the mir-17 cluster microRNA thereby preventing binding to cellular molecules with which the microRNA normally interacts, such that the normal biological activity of the mir-17 cluster microRNA is reduced or inhibited. Small molecules of the invention preferably have a molecular weight below 2,000 daltons, more preferably between 300 and 1,000 daltons, and still more preferably between 400 and 700 daltons. It is preferred that these small molecules are organic molecules.

Compounds that are identified as binding to a mir-17 cluster microRNA of the invention with an affinity constant less than or equal to 10 mM are considered particularly useful in the invention. Alternatively, any in vivo protein interaction detection system, for example, any two-hybrid assay may be utilized to identify compounds that interact with mir-17 cluster microRNA. Interacting compounds isolated by this method (or any other appropriate method) may, if desired, be further purified (e.g., by high performance liquid chromatography). Compounds isolated by any approach described herein may be used as therapeutics to treat a neoplasia in a human patient.

In addition, compounds that inhibit the expression of an mir-17 cluster microRNA whose expression is increased in a subject having a neoplasia are also useful in the methods of the invention. Any number of methods are available for carrying out screening assays to identify new candidate compounds that alter the expression of a mir-17 cluster microRNA.

The invention also includes novel compounds identified by the above-described screening assays. Optionally, such compounds are characterized in one or more appropriate animal models to determine the efficacy of the compound for the treatment of a neoplasia. Desirably, characterization in an animal model can also be used to determine the toxicity, side effects, or mechanism of action of treatment with such a compound. Furthermore, novel compounds identified in any of the above-described screening assays may be used for the treatment of a neoplasia in a subject. Such compounds are useful alone or in combination with other conventional therapies known in the art.

### Test Compounds and Extracts

In general, compounds capable of inhibiting the growth or proliferation of a neoplasia by decreasing the expression or biological activity of a mir-17 cluster microRNA (e.g., mir-17-5p or mir-17-20a) are identified from large libraries of either natural product or synthetic (or semi-synthetic) extracts or chemical libraries according to methods known in the art. Methods for making siRNAs are known in the art and are described in the Examples. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptide libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. EngL. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

In an embodiment of the invention, a high throughput approach can be used to screen different chemicals for their potency to affect the activity of a mir-17 cluster microRNA (e.g., mir-17-5p or mir-17-20a). A cell based sensor approach, as described in the Examples can be used to identify agents that inhibit expression of a mir-7 cluster microRNA (e.g., mir-17-5p or mir-17-20a). In one embodiment, the invention provides a method for identifying an agent that inhibits a neoplasia, the method comprising contacting a cell containing a sensor construct with an agent (polynucleotide, polypeptide, or small molecule), where the sensor construct contains a reporter gene linked to a site complementary to a microRNA of the mir-17 cluster; and measuring an alteration in the expression of the reporter gene relative to the expression of the reporter gene present in a control vector (e.g., a control vector not having a site complementary to a microRNA of the mir-17 cluster), wherein an alteration in the level of reporter expression identifies the agent as treating a neoplasia.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to alter the biological activity of a mir-17 cluster microRNA (e.g., mir-17-5p or mir-17-20a) variant, or fragment thereof, further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. if desired, compounds shown to be useful agents for the treatment of a neoplasm are chemically modified according to methods known in the art.

The present invention further provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neoplastic disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a neoplasia may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasoa, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### EXAMPLES

### Example 1

### MicroRNAs of the miR-17 Cluster are Upregulated in Cells Expressing High Levels of c-Myc

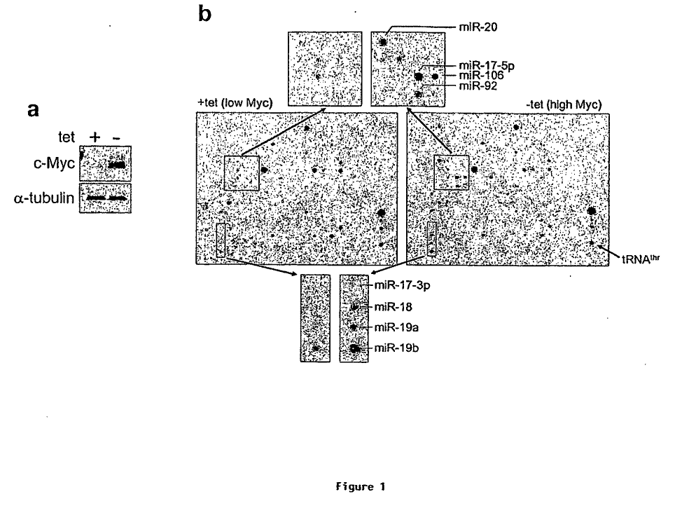

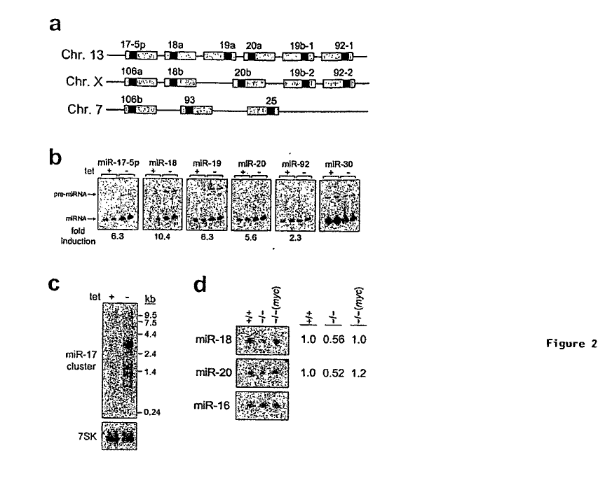

c-Myc is a helix-loop-helix leucine zipper transcription factor which regulates an estimated 10-15% of genes in the human and Drosophila genomes (Fernandez et al., Genes Dev 17, 1115-29 (2003); Li, Z. et al. Proc Natl Acad Sci USA 100, 8164-9 (2003); and Orian, A. et al., Genes Dev 17, 1101-14 (2003). Both c-Myc and miRNAs have been shown to influence cell proliferation and death and select miRNAs exhibit abnormal expression in human cancers (Cole et al., Oncogene 18, 2916-24 (1999); McManus et al., Semin Cancer Biol 13, 253-8 (2003)). To determine whether c-Myc regulates miRNA expression a spotted-oligonucleotide array capable of measuring the expression of 235 human, mouse, or rat miRNAs was generated and used to analyze a previously described human B-cell line, P493-6, that harbors a tetracycline-repressible c-MYC transgene (FIGS. 1A and 1B) (Pajic et al., Int J Cancer 87, 787-93 (2000)). miRNA expression profiles were analyzed in tet-treated (low c-Myc) or untreated (high c-Myc) cells. Six upregulated miRNAs were consistently observed in the high c-Myc state: miR5-17-5p, 18, 19, 20, 92, and 106. These miRNAs are encoded by three paralogous clusters located on chromosome 13 (the miR-17 cluster), the X chromosome (the miR-106a cluster), and chromosome 7 (the miR-106b cluster, FIG. 2A). Since the array did not detect upregulation of miR-25, also encoded by the miR-106b cluster, our analyses focused on the miR-17 and miR-106a clusters. Northern blotting confirmed that the miRNAs contained within these clusters were upregulated in the high c-Myc state (FIG. 2B). miR-17-3p, which has been reported to be expressed from the miR-17 cluster, was not detectable in P493-6 cells, suggesting that it may be a miRNA* sequence (FIG. 1B). The “*” denotes that the miR-17-3p strand is likely to be a passenger strand. During their biogenesis, microRNAs exist in a transient double-stranded form that resembles an siRNA. Only one strand of this duplex becomes a mature microRNA. The other strand, called the miRNA* or passenger strand, is rapidly degraded and rarely detectable in vivo. miRNAs are transcribed by RNA polymerase II as long primary transcripts (pri-miRNAs) that undergo sequential processing to produce mature miRNAs (Lee, et al., EMBO J. 23, 4051-60 (2004); Cai et al., RNA 10, 1957-66 (2004); Rodriguez et al., Genome Res 14, 1902-10 (2004); Lee et al. Nature 425, 415-9 (2003); Cullen et al., Mol Cell 16, 861-5 (2004)). Northern probes were designed to detect the pri-miRNA transcripts encompassing the miR-17 and miR-106a clusters. These probes were complementary to unique sequence immediately upstream of the first pre-miRNA hairpin in each cluster. The miR-17 cluster-specific probe detected three transcripts of approximately 3.2, 1.3, and 0.8 kilobases in size that were induced in the high c-Myc state (FIG. 2C). It has been reported that the miR-17 cluster is contained within an alternatively-spliced host transcript termed C13orf25 (Ota et al. Cancer Res 64, 3087-95 (2004)). The observed transcripts represented alternatively-spliced 5′-cleavage products of C13orf25 that remain following excision of pre-miRNAs. A similar probe complementary to sequence immediately upstream of the miR-106a cluster did not detect any transcripts in P493-6 cells. These data demonstrated that the miR-17 cluster is upregulated in the high c-Myc state.

In order to confirm that the regulation of the miR-17 cluster by c-Myc was not restricted to P493-6 cells, levels of miR-18 and miR-20 were examined in previously described wild-type rat fibroblasts (TGR), rat fibroblasts containing a homozygous deletion of c-myc (HO15.19), or null fibroblasts reconstituted with wild-type c-Myc (HO15.19-MYC) (Guo et al., Cancer Res 60, 5922-8 (2000); Mateyak et al., Cell Growth Differ 8, 1039-48 (1997)). miR-18 and miR-20 were expressed at approximately 50% of wild-type levels in the absence of c-Myc. Moreover, wild-type expression of these miRNAs was restored in the c-Myc-reconstituted null cells (FIG. 2D).

### Example 2

### The miR-17 Cluster is Directly Regulated by c-Myc

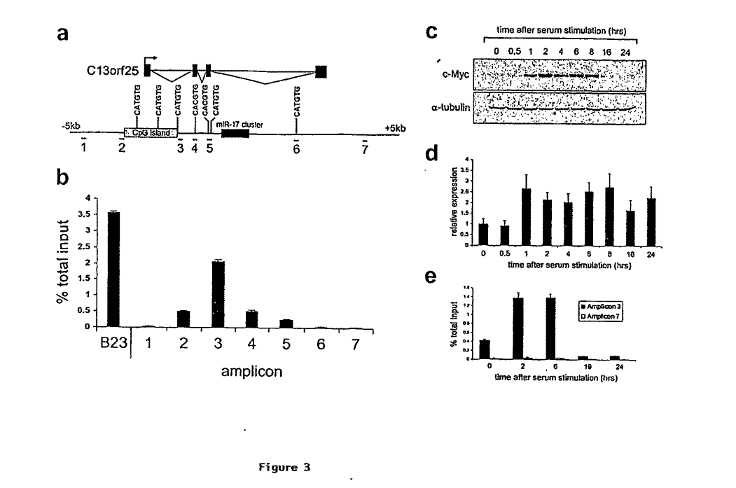

Chromatin immunoprecipitation (ChIP) was next performed in P493-6 cells to determine if human c-Myc binds directly to the miR-17 cluster genomic locus. First, 10 kb of sequence on chromosome 13 surrounding the miR-17 cluster was examined for putative c-Myc binding sites. c-Myc is known to bind to the canonical E-box sequence CACGTG as well as to non-canonical sequences including CATGTG (Zeller et al., Genome Biol 4, R69 (2003)). Seven putative binding sites matching these sequences were identified. Four of these sites were conserved between human and mouse and located within a 30 base-pair window of at least 65% nucleotide identity between these species (FIG. 3A, labeled in gray). Real-time PCR amplicons were designed to assay for all putative binding sites (both conserved and nonconserved) in ChIP samples. Background signals were very low at all tested amplicons in negative control samples immunoprecipitated without antibody or with an antibody directed against hepatocyte growth factor (HGF) which is not expressed in these cells Clear evidence for in vivo association of c-Myc with a region containing a conserved CATGTG sequence 1480 nucleotides upstream of miR-17-5p was obtained (FIG. 3B, amplicon 3). This site is located in intron 1 of the C13orf25 transcript. c-Myc is known to frequently bind within the first intron of its transcriptional target genes (Zeller et al., supra). Since amplicons between nucleotides −1500 to −3280 were not designed due to the presence of a CpG island which prevented efficient amplification, the possibility that c-Myc also binds within this region cannot be ruled out. These data demonstrated that c-Myc binds directly to the miR-17 cluster genomic locus, providing strong evidence that these miRNAs are directly regulated by this transcription factor.

Seven putative binding sites in the vicinity of the miR-106a cluster were also identified and assayed for c-Myc binding. No ChIP signals were observed, consistent with the northern data demonstrating an absence of detectable transcripts produced from this locus in P493-6 cells.

The behavior of the miR-17 cluster was also examined during serum stimulation in primary human fibroblasts. Serum deprivation followed by serum stimulation of fibroblasts results in a transient induction of c-Myc (Matsumura et al., Cell Cycle 2, 333-8 (2003)). (FIG. 3C). Real-time PCR analysis demonstrated that expression of the miR-17 host transcript is induced with similar kinetics under these conditions (FIG. 3D). Consistent with the behavior of other known c-Myc target genes, expression levels remain elevated after c-Myc levels decrease (Zeller et al., J Biol Chem 276, 48285-91 (2001)). Furthermore, ChIP analysis demonstrated that association of c-Myc with the miR-17 genomic locus mirrors c-Myc expression and coincides with induction of expression of the miRNA cluster (FIG. 3E). These results indicated that the miR-17 cluster is directly regulated by c-Myc and demonstrated that induction of these miRNAs is a physiologic response to growth stimuli.

### Example 3

### miR-17-5p and miR-20a Negatively Regulate E2F1 Translation

To study the functional consequences of induction of the miR-17 cluster by c-Myc, mRNAs that are predicted targets of these miRNAs were examined. The transcription factor E2F1, predicted to be regulated by miR-17-5p and miR-20a (Lewis et al., Cell 115, 787-98 (2003), was initially chosen for further analysis. E2F1 expression promotes G1 to S phase progression in mammalian cells by activating genes involved in DNA replication and cell cycle control (Bracken et al., Trends Biochem Sci 29, 409-17 (2004)). Expression of the E2F1 gene has been demonstrated to be induced by c-Myc (Leone et al., Nature 387, 422-6 (1997); Fernandez et al. Genes Dev 17, 1115-29 (2003)). c-Myc expression is also induced by E2F1, revealing a putative positive feedback circuit (Matsumura et al., Cell Cycle 2, 333-8 (2003). Negative regulation of E2F1 translation by miR-17-5p and miR-20a was hypothesized to provide a mechanism to dampen this reciprocal activation, thus promoting tightly-controlled expression of these gene products.

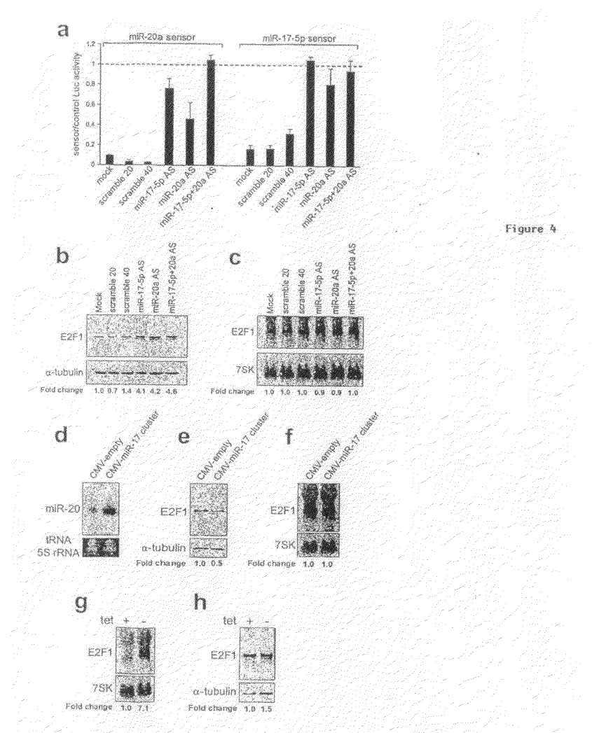

To determine if E2F1 is a target of miR-17-5p and miR-20a, HeLa cells that naturally express the miR-17 cluster were utilized. 2′-O-methyl antisense oligoribonucleotides, which have been demonstrated to block miRNA function (Meister et al., RNA 10, 544-50 (2004); Hutvagner et al., PLoS Biol 2, E98 (2004)), were designed to inhibit miR-17-5p and miR-20a. To monitor the degree of inhibition of these miRNAs, sensor constructs with sites perfectly complementary to miR-17-5p or miR-20a in the 3′ untranslated region (UTR) of firefly luciferase were generated. When introduced into HeLa cells, these constructs exhibited an 80-90% reduction in luciferase activity as compared to control constructs containing the reverse-complement sequence of the miRNA binding sites, demonstrating efficient downregulation by endogenous miR-17-5p and miR-20a. Co-transfection of these plasmids with miR-17-5p or miR-20a antisense oligonucleotides, but not scrambled oligonucleotides, enhanced expression of the sensor constructs, indicating inhibition of these miRNAs (FIG. 4A). Because of nucleotide similarity between miR-17-5p and miR-20a, both were inhibited by antisense oligonucleotides directed against either miRNA. Transfection of miR-17-5p and miR-20a antisense oligonucleotides, but not scrambled oligonucleotides, resulted in an approximately 4-fold increase in E2F1 protein levels without altering E2F1 mRNA abundance (FIGS. 4B and 4C).

The consequence of overexpression of the miR-17 cluster on E2F1 expression was also determined. The entire miR-17 cluster and approximately 150 nucleotides of flanking sequence were cloned into a mammalian expression vector. When transfected into HeLa cells, this construct (CMV-miR-17 cluster) produced the appropriately processed miRNAs, as assessed by northern blotting (FIG. 4D). Transient overexpression of these miRNAs resulted in a 50% decrease in E2F1 protein levels (FIG. 4E) without affecting E2F1 mRNA abundance (FIG. 4F).

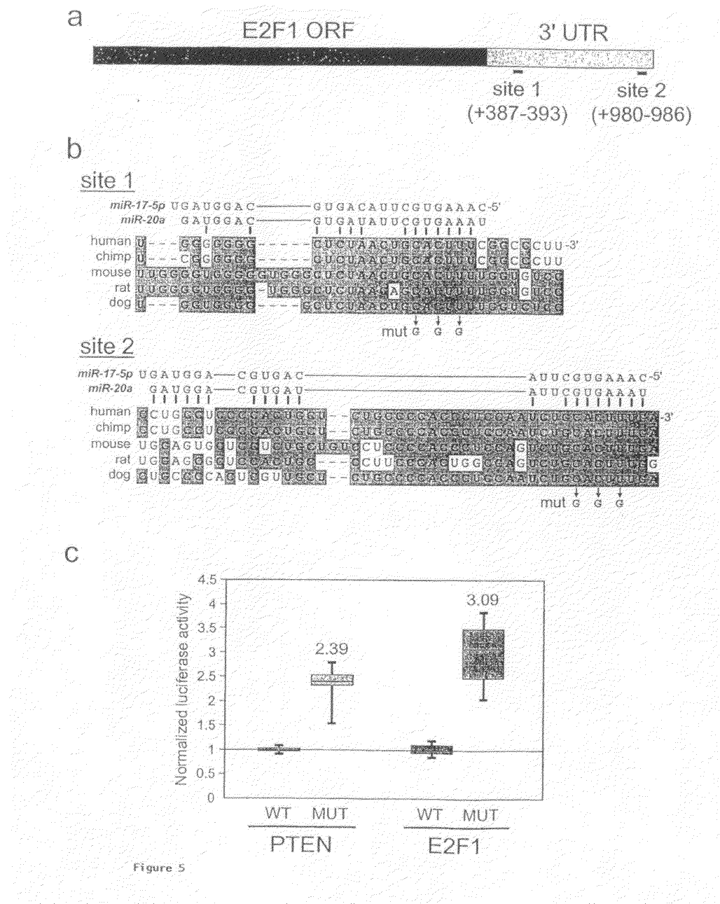

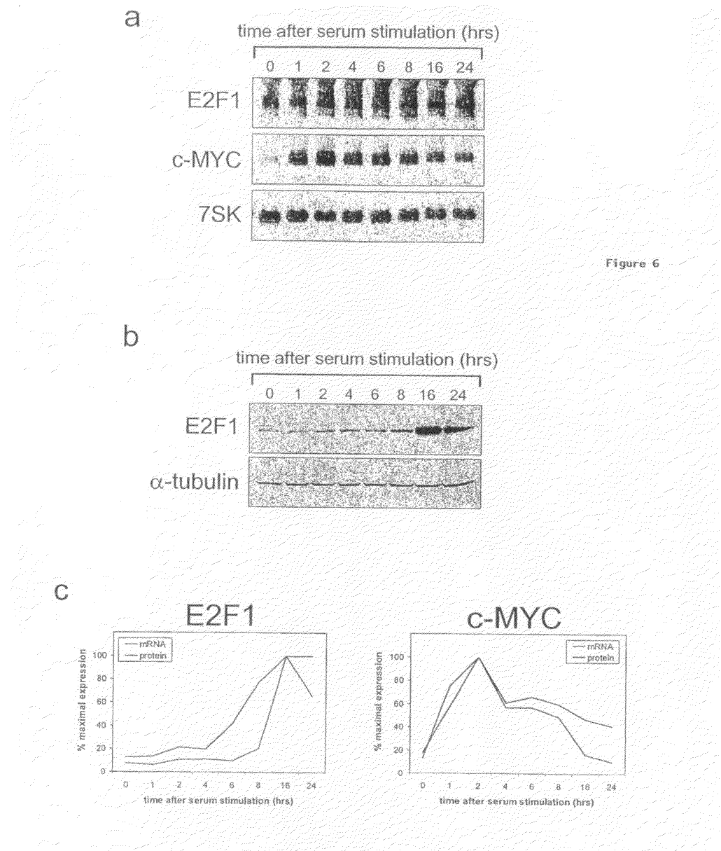

To demonstrate that miR-17-5p and miR-20a directly regulate E2F1 expression, luciferase reporter constructs containing a portion of the E2F1 3′UTR were generated and mutations were introduced into the predicted miRNA-binding sites (FIGS. 5A and 5B). The mutant construct yielded approximately 3-fold higher luciferase expression compared to the wild-type construct when transfected into HeLa cells, providing evidence that the endogenously expressed miRNAs decrease E2F1 expression by recognizing these sites (FIG. 5C). Finally, E2F1 mRNA and protein levels were examined in P493-6 cells with high and low c-Myc expression (and consequently high and low expression of the miR-17 cluster). Consistent with reported data (Leone et al., Nature 387, 422-6 (1997); Fernandez et al., Genes Dev 17, 1115-29 (2003)), c-Myc potently induced E2F1 mRNA (FIG. 4G). Remarkably, E2F1 protein levels were only modestly induced under these conditions, suggesting a greatly reduced translational yield (FIG. 4H). Taken together with the results from HeLa cells, without wishing to be bound by theory, it is likely that miR-17-5p and miR-20a limit c-Myc-mediated induction of E2F1 expression, preventing uncontrolled reciprocal activation of these gene products. Since E2F1 protein is known to accumulate late in G1 and 10 c-Myc and consequently the miR-17 cluster is activated early in G1 (Bracken et al., Trends Biochem Sci 29, 409-17 (2004); Matsumura et al., Cell Cycle 2, 333-8 (2003)), it is likely that E2F1 translational efficiency is decreased, but not completely inhibited by these miRNAs during normal cell-cycle progression. Consistent with a dampened translational efficiency, E2F1 protein accumulation is delayed relative to E2F1 mRNA induction during a serum stimulation time-course in primary fibroblasts. In contrast, c-Myc protein levels closely mirrored mRNA levels under these conditions (FIGS. 6A-6C). As several other documented c-Myc target genes are also predicted targets of the miR-17 cluster (e.g. RPS6KA5, BCL11B, PTEN, and HCF2) (Zeller, et al., Genome Biol 4, R69 (2003); Lewis et al., Cell 115, 787-98 (2003)), a widespread mechanism may exist through which c-Myc and other transcription factors precisely control expression of target genes by simultaneously activating their transcription and limiting their translation.

These results identified miRNAs as novel c-Myc targets, expanding the known classes of transcripts within the c-Myc target gene network. Furthermore, they suggested that the miR-17 cluster, by decreasing E2F1 expression, tightly regulates c-Myc-mediated cellular proliferation. In this context, these miRNAs are likely to exhibit tumor suppressor activity. Accordingly, loss-of-heterozygosity of the chromosomal region encompassing the miR-17 cluster (13q31) has been observed in human malignancies (Lin et al., Eur J Cancer 35, 1730-4 (1999)). Amplification of this region and overexpression of C13orf25, the host transcript of the miR-17 cluster, has been described in diffuse large B-cell lymphoma (Ota et al., Cancer Res 64, 3087-95 (2004)) and miR-19a and miR-92-1 have been shown to be upregulated in B cell chronic lymphocytic leukemia (Calin et al., Proc Natl Acad Sci USA 101, 11755-60 (2004)). These observations suggest that these miRNAs may also possess oncogenic activity. It is thus likely that these miRNAs influence cell proliferation and tumorigenesis in a cell-type-specific manner, depending on the mileu of target mRNAs that are expressed. The results described here provide an experimental framework for further functional dissection of this miRNA cluster in order to fully delineate its role in normal cellular physiology and malignancy.

These data demonstrate that the miR-17 cluster is transcriptionally activated by c-Myc, an oncogenic transcription factor that is frequently dysregulated in cancer cells (O'Donnell et al., 2005). Furthermore, they show that the critical cell-cycle regulatory factor E2F1 is downregulated by these microRNAs (miRNAs). Further studies described below indicated that another key regulator of the cell-cycle, the protein p21, is regulated by two miRNAs in the miR-17 cluster: miR-17-5p and miR-20a. Downregulation of p21 by these miRNAs is expected to profoundly influence normal cell-cycle control and promote proliferation of cancer cells.

Transition through the G1/S and G2/M checkpoints is controlled by cyclin-dependent kinases (cdks) which phosphorylate target proteins and promote cell-cycle progression. p21 is the founding member of a class of proteins which bind to cdks and arrest the cell cycle (el-Deiry et al., Cell 75, 817-825, 1993). The p53 tumor suppressor is a transcription factor that senses DNA damage and subsequently arrests the cell cycle and/or induces cell death. Loss of function of p53 is one of the most common abnormalities in human cancer cells (Vogelstein et al., Nature 408, 307-310, 2000). The first transcriptional target of p53 that was identified was p21. It was subsequently demonstrated that the G1/S checkpoint arrest that p53 induces in response to DNA damage absolutely requires induction of p21 (Waldman et al., Cancer Res 55, 5187-5190, 1995). Fully efficient p53-mediated arrest at the G2/M checkpoint also requires p21. Thus, any cellular perturbation that prevents upregulation of p21 in response to DNA damage is expected to partially phenocopy p53 loss-of-function, leading to enhanced cellular proliferation and tumorigenesis.

### Example 4

### Expression of the miR-17 Cluster Downregulated p21

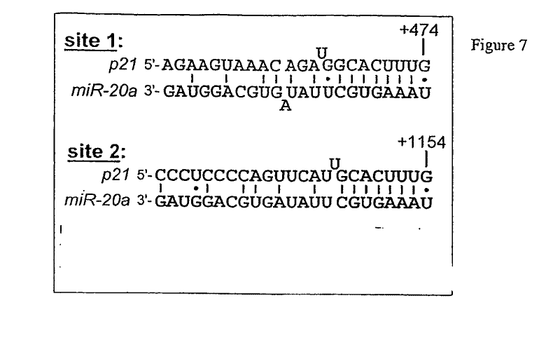

Because the miR-17 cluster is activated by c-Myc and is frequently overexpressed in cancer cells, it might promote tumorigenesis by downregulating p21. To test this hypothesis, the sequence of the p21 transcript was searched for putative binding sites for the miR-17 cluster miRNAs. Two sites in the p21 3′ untranslated region (3′ UTR) were identified having perfect complementarity to nucleotides 2-8 of miR-17-5p and miR-20a (FIG. 7). Binding of this portion of a miRNA to an mRNA target is believed to be necessary and sufficient for miRNA-mediated regulation (Lewis et al., Cell 115, 787-798, 2003). Site 1 was also recognized in a genome-wide search for the predicted binding sites for all known miRNAs (Lewis et al., Cell 120, 15-20, 2005). Nevertheless, site 1 is nonfunctional, whereas site 2, which has never been described prior to this report, is functional.

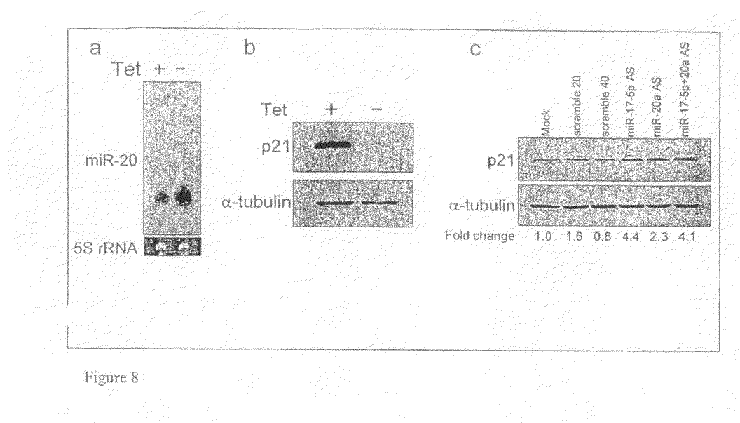

In order to determine if p21 is a bona fide target of miR-17-5p and miR-20a, the expression of this protein was measured following overexpression or inhibition of these miRNAs. To overexpress the miR-17 cluster, a novel HeLa cell line was generated in which these miRNAs were placed under the control of a tetracycline (tet)-repressible promoter. This cell line is referred to as TRE-miR17HeLa. When tet is withdrawn, the miR-17 cluster is induced ˜5-fold as demonstrated by northern blot (FIG. 8A). Upon induction of the miR-17 cluster, a dramatic reduction in p21 protein abundance was observed (FIG. 8B). miR-17-5p and miR-20a were also inhibited using 2′-O-methyl antisense oligonucleotides in native HeLa cells. This led to an ˜4-fold increase in p21 protein abundance. Transfection with increasing concentration of scrambled oligo did not affect p21 protein levels, demonstrating the specificity of this finding (FIG. 8C). These data indicate that the miR-17 cluster downregulated p21.

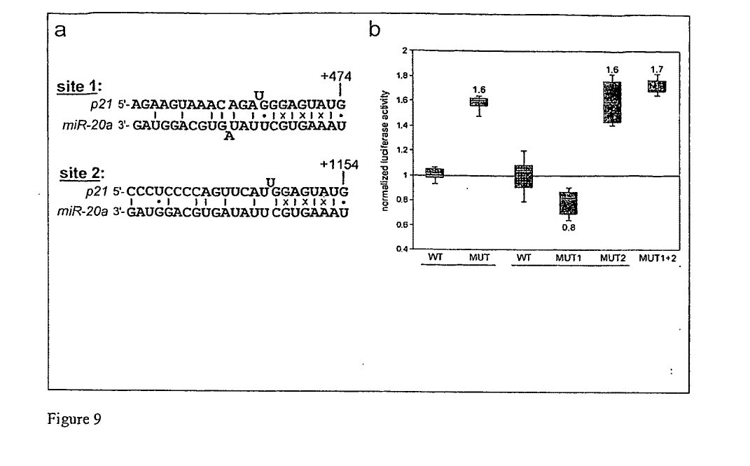

In order to demonstrate that the regulation of p21 by miR-17-5p and miR-20a is direct, luciferase reporter assays were utilized. The entire 3′ UTR of p21 was cloned into the pGL3-control plasmid (Promega), placing it downstream of the firefly luciferase open reading frame. Mutations were then introduced into the putative miRNA binding sites (FIG. 9A). If the miRNAs inhibit translation of the p21 transcript by recognizing these sites, the mutant constructs will produce higher amounts of luciferase activity compared to the wild-type construct when introduced into cells that express the miRNAs. Both wild-type and mutant plasmids were transfected into HeLa cells which naturally express the mir-17 cluster. As a control for transfection efficiency, p21 firefly luciferase reporter constructs were co-transfected with a plasmid that constitutively expresses renilla luciferase. Renilla luciferase activity was used to normalize all experimental measurements. As a positive control for these experiments, previously described reporter constructs for PTEN, a miR-19 target, was used (O'Donnell et al., Nature 435, 839-843, 2005). As shown in FIG. 9B, the p21 reporter with a mutation in site I (MUT1) did not exhibit increased luciferase activity, demonstrating that site 1 is unlikely to be a functional miRNA binding site. In contrast, mutation of site 2 (MUT2) led to a highly reproducible increase in luciferase activity. Combining a site 1 mutation with a site 2 mutation (MUT1+2) did not further increase luciferase activity. Taken together with the expression data shown in FIG. 8, these results provide compelling evidence that p2p is directly downregulated by miR-17-5p and miR-20a. These findings provide critical molecular insight into the mechanism through which the miR-17 cluster promotes tumorigenesis. Additionally, they further support the development of a therapeutic strategy involving inhibition of these miRNAs to treat diverse human cancers.

The above results were obtained using the following methods and materials.

### Tissue Culture

P493-6 cells (from D. Eick) were cultured in RPM1 1640 media supplemented with 10% fetal bovine serum (FBS) and penicillin-streptomycin (Pen-Strep). To repress c-MYC expression, cells were grown in the presence of 0.1 μg/ml tetracycline (Sigma) for 72 hours. TGR-1 cells (wild-type rat fibroblasts) and HO15.19 cells (c-MYC −/− rat fibroblast) were a gift from J. Sedivy. Rat cells, HeLa cells, and primary human fibroblasts (obtained from ATCC) were grown in Dulbecco's Modified Eagle's Medium (DMEM) with 10% FBS and Pen-Strep. For serum stimulation experiments, primary fibroblasts were grown in DMEM with 0.1% FBS for 48 hours. DMEM containing 10% FBS was then added and cells were harvested at indicated time-points.

miRNA Expression

miRNA expression arrays were generated and probed essentially as described (Krichevsky et al., RNA 9, 1274-81 (2003)) with the following modifications. Oligonucleotide probes (each a concatemerized triple repeat of sequence antisense to a mature miRNA) were synthesized and spotted at a concentration of 10 μM on GeneScreen Plus membranes (Perkin Elmer) with a 96-pin spotter (V&P Scientific). In addition to 235 miRNA probes, 25 probes containing 3 mismatches were spotted to assess hybridization specificity. These probes produced significantly less signal intensity (in general, an approximately 10-fold decrease) compared to wild-type probes. A probe complementary to tRNA^{thr} controlled for hybridization efficiency. All probe sequences are provided in Table 1 below.

TABLE 1

| miRNA | Probe sequence |
|---|---|
| let-7a | AACTATACAACCTACTACCTCAAACTATACAACCTACTACCTCAAACTATACAACCTACTACCTCA |
| (1-3) | |
| let-7b | AACCACACAACCTACTACCTCAAACCACACAACCTACTACCTCAAACCACACAACCTACTACCTCA |
| let-7c | AACCATACAACCTACTACCTCAAACCATACAACCTACTACCTCAAACCATACAACCTACTACCTCA |
| let-7d | ACTATGCAACCTACTACCTCTACTATGCAACCTACTACCTCTACTATGCAACCTACTACCTCT |
| let-7e | ACTATACAACCTCCTACCTCAACTATACAACCTCCTACCTCAACTATACAACCTCCTACCTCA |
| let-7f | AACTATACAATCTACTACCTCAAACTATACAATCTACTACCTCAAACTATACAATCTACTACCTCA |
| (1; 2) | |
| let-7g | TACTGTACAAACTACTACCTCATACTGTACAAACTACTACCTCATACTGTACAAACTACTACCTCA |
| let-7h | AACTGTACACACTACTACCTCAAACTGTACACACTACTACCTCAAACTGTACACACTACTACCTCA |
| let-7l | AGCACAAACTACTACCTCAAGCACAAACTACTACCTCAAGCACAAACTACTACCTCA |
| miR-1b | TTACATACTTCTTTACATTCCATTACATACTTCTTTACATTCCATTACATACTTCTTTACATTCCA |
| miR-1c | GTACATACTTCTTTACATTCCAGTACATACTTCTTTACATTCCAGTACATACTTCTTTACATTCCA |
| miR-1d | AATACATACTTCTTTACATTCCAAATACATACTTCTTTACATTCCAAATACATACTTCTTTACATTCCA |
| miR-7 | ACAACAAAATCACTAGTCTTCCAACAACAAAATCACTAGTCTTCCAACAACAAAATCACTAGTCTTCCA |
| miR-9 | TCATACAGCTAGATAACCAAAGATCATACAGCTAGATAACCAAAGATCATACAGCTAGATAACCAAAGA |
| miR-10a | CACAAATTCGGATCTACAGGGTACACAAATTCGGATCTACAGGGTACACAAATTCGGATCTACAGGGTA |
| miR-10b | ACACAAATTCGGTTCTACAGGGACACAAATTCGGTTCTACAGGGACACAAATTCGGTTCTACAGGG |
| miR-15a | CACAAACCATTATGTGCTGCTACACAAACCATTATGTGCTGCTACACAAACCATTATGTGCTGCTA |
| miR-15b | TGTAAACCATGATGTGCTGCTATGTAAACCATGATGTGCTGCTATGTAAACCATGATGTGCTGCTA |
| miR-16 | CGCCAATATTTACGTGCTGCTACGCCAATATTTACGTGCTGCTACGCCAATATTTACGTGCTGCTA |
| (1, 2) | |
| miR-17-3p | ACAAGTGCCTTCACTGCAGTACAAGTGCCTTCACTGCAGTACAAGTGCCTTCACTGCAGT |
| mir-17-3p | ACAAGTGCCCTCACTGCAGTACAAGTGCCCTCACTGCAGTACAAGTGCCCTCACTGCAGT |
| (mouse) | |
| miR-18 | TATCTGCACTAGATGCACCTTATATCTGCACTAGATGCACCTTATATCTGCACTAGATGCACCTTA |
| miR-19a | TCAGTTTTGCATAGATTTGCACATCAGTTTTGCATAGATTTGCACATCAGTTTTGCATAGATTTGCACA |
| miR-19b | TCAGTTTTGCATGGATTTGCACATCAGTTTTGCATGGATTTGCACATCAGTTTTGCATGGATTTGCACA |
| (1, 2) | |
| miR-20 | CTACCTGCACTATAAGCACTTTACTACCTGCACTATAAGCACTTTACTACCTGCACTATAAGCACTTTA |
| miR-21 | TCAACATCAGTCTGATAAGCTATCAACATCAGTCTGATAAGCTATCAACATCAGTCTGATAAGCTA |
| miR-22 | ACAGTTCTTCAACTGGCAGCTTACAGTTCTTCAACTGGCAGCTTACAGTTCTTCAACTGGCAGCTT |
| miR-23a | GGAAATCCCTGGCAATGTGATGGAAATCCCTGGCAATGTGATGGAAATCCCTGGCAATGTGAT |
| miR-23b | GTGGTAATCCCTGGCAATGTGATGTGGTAATCCCTGGCAATGTGATGTGGTAATCCCTGGCAATGTGAT |
| miR-24 | CTGTTCCTGCTGAACTGAGCCACTGTTCCTGCTGAACTGAGCCACTGTTCCTGCTGAACTGAGCCA |
| (1, 2) | |
| miR-25 | TCAGACCGAGACAAGTGCAATGTCAGACCGAGACAAGTGCAATGTCAGACCGAGACAAGTGCAATG |
| miR-26a | AGCCTATCCTGGATTACTTGAAAGCCTATCCTGGATTACTTGAAAGCCTATCCTGGATTACTTGAA |
| miR-26b | AACCTATCCTGAATTACTTGAAAACCTATCCTGAATTACTTGAAAACCTATCCTGAATTACTTGAA |
| miR-27a | AGCGGAACTTAGCCACTGTGAAAGCGGAACTTAGCCACTGTGAAAGCGGAACTTAGCCACTGTGAA |
| miR-27b | CAGAACTTAGCCACTGTGAACAGAACTTAGCCACTGTGAACAGAACTTAGCCACTGTGAA |
| miR-28 | CTCAATAGACTGTGAGCTCCTTCTCAATAGACTGTGAGCTCCTTCTCAATAGACTGTGAGCTCCTT |
| miR-29a | AACCGATTTCAGATGGTGCTAGAACCGATTTCAGATGGTGCTAGAACCGATTTCAGATGGTGCTAG |
| miR-29b | AACACTGATTTCAAATGGTGCTAAACACTGATTTCAAATGGTGCTAAACACTGATTTCAAATGGTGCTA |
| miR-29c | TAACCGATTTCAAATGGTGCTAgTAACCGATTTCAAATGGTGCTAgTAACCGATTTCAAATGGTGCTAg |
| miR-30a-s | GCTTCCAGTCGAGGATGTTTACAGCTTCCAGTCGAGGATGTTTACAGCTTCCAGTCGAGGATGTTTACA |
| miR-30a-as | GCTGCAAAGATCCGACTGAAAGGCTGCAAACATCCGACTGAAAGGCTGCAAACATCCGACTGAAAG |
| miR-30b | GCTGAGTGTAGGATGTTTACAGCTGAGTGTAGGATGTTTACAGCTGAGTGTAGGATGTTTACA |
| miR-30c | GCTGAGAGTGTAGGATGTTTACAGCTGAGAGTGTAGGATGTTTACAGCTGAGAGTGTAGGATGTTTACA |
| miR-30d | CTTCCAGTCGGGGATGTTTACACTTCCAGTCGGGGATGTTTACACTTCCAGTCGGGGATGTTTACA |
| miR-30e | TCCAGTCAAGGATGTTTACATCCAGTCAAGGATGTTTACATCCAGTCAAGGATGTTTACA |
| miR-31 | CAGCTATGCCAGCATCTTGCCCAGCTATGCCAGCATCTTGCCCAGCTATGCCAGCATCTTGCC |
| miR-32 | GCAACTTAGTAATGTGCAATAGCAACTTAGTAAATGTGCAATAGCAACTTAGTAATGTGCAATA |
| miR-33 | CAATGCAACTACAATGCACCAATGCAACTACAATGCACCAATGCAACTACAATGCAC |
| mir-33b | CAATGCAACAGCAATGCACCAATGCAACAGCAATGCACCAATGCAACAGCAATGCAC |
| miR-34 | ACAACCAGCTAAGACACTGCCAACAACCAGCTAAGACACTGCCAACAACCAGCTAAGACACTGCCA |
| miR-17-5p | ACTACCTGCACTGTAAGCACTTTGACTACCTGCACTGTAAGCACTTTGACTACCTGCACTGTAAGCACTTTG |
| miR-92 | ACAGGCCGGGACAAGTGCAATAACAGGCCGGGACAAGTGCAATAACAGGCCGGGACAAGTGCAATA |
| miR-93 | CTACCTGCACGAACAGCACTTTCTACCTGCACGAACAGCACTTTCTACCTGCACGAACAGCACTTT |
| miR-94 | ATCTGCACTGTCAGCACTTTATCTGCACTGTCAGCACTTTATCTGCACTGTCAGCACTTT |
| miR-95 | TGCTCAATAAATACCCGTTGAATGCTCAATAAATACCCGTTGAATGCTCAATAAATACCCGTTGAA |
| miR-96 | GCAAAAATGTGCTAGTGCCAAAGCAAAAATGTGCTAGTGCCAAAGCAAAAATGTGCTAGTGCCAAA |
| miR-98 | AACAATACAACTTACTACCTCAAACAATACAACTTACTACCTCAAACAATACAACTTACTACCTCA |
| miR-99a | ACAAGATCGGATCTACGGGTACAAGATCGGATCTACGGGTACAAGATCGGATCTACGGGT |
| miR-99b | CGCAAGGTCGGTTCTACGGGTGCGCAAGGTCGGTTCTACGGGTGCGCAAGGTCGGTTCTACGGGTG |
| miR-100 | CACAAGTTCGGATCTACGGGTTCACAAGTTCGGATCTACGGGTTCACAAGTTCGGATCTACGGGTT |
| miR-101 | TCAGTTATCACAGTACTGTATCAGTTATCACAGTACTGTATCAGTTATCACAGTACTGTA |
| miR-103 | TCATAGCCCTGTACAATGCTGCTTCATAGCCCTGTACAATGCTGCTTCATAGCCCTGTACAATGCTGCT |
| miR-104 | TAGCTTATCAGACTGATGTTGATAGCTTATCAGACTGATGTTGATAGCTTATCAGACTGATGTTGA |
| miR-105 | ACAGGAGTCTGAGCATTTGAACAGGAGTCTGAGCATTTGAACAGGAGTCTGAGCATTTGA |
| miR-106 | GCTACCTGCACTGTAAGCACTTTTGCTACCTGCACTGTAAGCACTTTTGCTACCTGCACTGTAAGCACTTTT |
| miR-107 | TGATAGCCCTGTACAATGCTGCTTGATAGCCCTGTACAATGCTGCTTGATAGCCCTGTACAATGCTGCT |
| miR-108 | AATGCCCCTAAAAATCCTTATAATGCCCCTAAAAATCCTTATAATGCCCCTAAAAATCCTTAT |
| miR-109 | GCGCAGGCCGACTCGACCAGGCGCAGGCCGACTCGACCAGGCGCAGGCCGACTCGACCAG |
| miR-110 | CGACGTCGGCCGCTCGACGACGTCGGCCGCTCGACGACGTCGGCCGCTCGA |
| miR-112 | TTCCGTGGATGTCAGGACCTTCCGTGGATGTCAGGACCTTCCGTGGATGTCAGGACC |
| miR-113 | TGGTGGGCGCACCAGGGCTCGATGGTGGGCGCACCAGGGCTCGATGGTGGGCGCACCAGGGCTCGA |
| miR-114 | CGCCAATATTTACGTGCAGCTACGCCAATATTTACGTGCAGCTACGCCAATATTTACGTGCAGCTA |
| miR-115 | TTCCAGCTCCGCTTCATTCCAGCTCCGCTTCATTCCAGCTCCGCTTCA |
| miR-116 | CAGCGTTCGTCCTGAGCCAGGATCAACAGCGTTCGTCCTGAGCCAGGATCAACAGCGTTCGTCCTGAGCCAGGATCAA |
| miR-117 | AACTGTTCCTGCTGAAAACTGTTCCTGCTGAAAACTGTTCCTGCTGAA |
| miR-118 | ATCCTACACTCAAGGCATATCCTACACTCAAGGCATATCCTACACTCAAGGCAT |
| miR-119 | ATGGTAATCCCTGGCAATATGGTAATCCCTGGCAATATGGTAATCCCTGGCAAT |
| miR-120 | ACACGCTGCGTCCCGCCACACGCTGCGTCCCGCCACACGCTGCGTCCCGCC |
| miR-121 | TTCGGGAAGGGATCAGGTGGTTCATTCGGGAAGGGATCAGGTGGTTCATTCGGGAAGGGATCAGGTGGTTCA |
| miR-122a | ACAAACACCATTGTCACACTCCAACAAACACCAATGTCACACTCCAACAAACACCATTGTCACACTCCA |
| miR-122b | TCAAACACCATTGTCACACTCCATCAAACACCATTGTCACACTCCATCAAACACCATTGTCACACTCCA |
| miR-123/ | CGCGTACCAAAAGTAATAATGCGCGTACCAAAAGTAATAATGCGCGTACCAAAAGTAATAATG |
| miR-126-s | |
| miR-126/ | GCATTATTACTCACGGTACGAGCATTATTACTCACGGTACGAGCATTATTACTCACGGTACGA |
| miR-123-as | |
| miR-124a | TGGCATTCACCGCGTGCCTTAATGGCATTCACCGCGTGCCTTAATGGCATTCACCGCGTGCCTTAA |
| miR-124b | GCATTCACCCGCGTGCCTTAAGCATTCACCCGCGTGCCTTAAGCATTCACCCGCGTGCCTTAA |
| miR-125a | CACAGGTTAAAGGGTCTCAGGGACACAGGTTAAAGGGTCTCAGGGACACAGGTTAAAGGGTCTCAGGGA |
| miR-125b | TCACAAGTTAGGGTCTCAGGGATCACAAGTTAGGGTCTCAGGGATCACAAGTTAGGGTCTCAGGGA |
| miR-127 | AGCCAAGCTCAGACGGATCCGAAGCCAAGCTCAGACGGATCCGAAGCCAAGCTCAGACGGATCCGA |
| miR-128 | AAAAGAGACCGGTTCACTGTGAAAAAGAGACCGGTTCACTGTGAAAAAGAGACCGGTTCACTGTGA |
| miR-129 | GCAAGCCCAGACCGAAAAAAGGCAAGCCCAGACCGAAAAAAGGCAAGCCCAGACCGAAAAAAG |
| miR-129b | AGCAAGCCCAGACCGCAAAAAGAGCAAGCCCAGACCGCAAAAAGAGCAAGCCCAGACCGCAAAAAG |
| miR-130 | GCCCTTTTAACATTGCACTGGCCCTTTTAACATTGCACTGGCCCTTTTAACATTGCACTG |
| mir-130b | GCCCTTTCATCATTGCACTGGCCCTTTCATCATTGCACTGGCCCTTTCATCATTGCACTG |
| miR-131 | ACTTTCGGTTATCTAGCTTTAACTTTCGGTTATCTAGCTTTAACTTTCGGTTATCTAGCTTTA |
| miR-132 | gCGACCATGGCTGTAGACTGTTAgCGACCATGGCTGTAGACTGTTAgCGACCATGGCTGTAGACTGTTA |
| miR-133 | ACAGCTGGTTGAAGGGGACCAAACAGCTGGTTGAAGGGGACCAAACAGCTGGTTGAAGGGGACCAA |
| miR-134 | TCCCTCTGGTCAACCAGTCACATCCCTCTGGTCAACCAGTCACATCCCTCTGGTCAACCAGTCACA |
| miR-134 | CCCCTCTGGTCAACCAGTCACACCCCTCTGGTCAACCAGTCACACCCCTCTGGTCAACCAGTCACA |
| (mouse) | |
| miR-135 | ATCACATAGGAATAAAAAGCCATAATCACATAGGAATAAAAAGCCATAATCACATAGGAATAAAAAGCCATA |
| miR-136 | TCCATCATCAAAACAAATGGAGTTCCATCATCAAAACAAATGGAGTTCCATCATCAAAACAAATGGAGT |
| miR-137 | CTACGCGTATTCTTAAGCAATACTACGCGTATTCTTAAGCAATACTACGCGTATTCTTAAGCAATA |
| miR-138 | GATTCACAACACCAGCTGATTCACAACACCAGCTGATTCACAACACCAGCT |
| miR-139 | AGACACGTGCACTGTAGAAGACACGTGCACTGTAGAAGACACGTGCACTGTAGA |
| miR-140s | CTACCATAGGGTAAAACCACTCTACCATAGGGTAAAACCACTCTACCATAGGGTAAAACCACT |
| miR-140as | TCCGTGGTTCTACCCTGTGGTATCCGTGGTTCTACCCTGTGGTATCCGTGGTTCTACCCTGTGGTA |
| miR-141 | CCATCTTTACCAGACAGTGTTCCATCTTTACCAGACAGTGTTCCATCTTTACCAGACAGTGTT |
| miR-142s | GTAGTGCTTTCTACTTTATGGTAGTGCTTTCTACTTTATGGTAGTGCTTTCTACTTTATG |
| miR-142as | CCATAAAGTAGGAAACACTACACCATAAAGTAGGAAACACTACACCATAAAGTAGGAAACACTACA |
| miR-143 | TAAGAGCTACAGTGCTTCATCTCATAAGAGCTACAGTGCTTCATCTCATAAGAGCTACAGTGCTTCATCTCA |
| miR-144 | CTAGTACATCATCTATACTGTACTAGTACATCATCTATACTGTACTAGTACATCATCTATACTGTA |
| miR-145 | AAGGGATTCCTGGGAAAACTGGACAAGGGATTCCTGGGAAAACTGGACAAGGGATTCCTGGGAAAACTGGAC |
| miR-146 | AAACCCATGGAATTCAGTTCTCAAAACCCATGGAATTCAGTTCTCAAAACCCATGGAATTCAGTTCTCA |
| miR-146 | TAACCCATGGAATTCAGTTCTCATAACCCATGGAATTCAGTTCTCATAACCCATGGAATTCAGTTCTCA |
| (mouse) | |
| miR-147 | GGCAGAAGCATTTCCACACACGGCAGAAGCATTTCCACACACGGCAGAAGCATTTCCACACAC |
| miR-148 | ACAAAGTTCTGTAGTGCACTGAACAAAGTTCTGTAGTGCACTGAACAAAGTTCTGTAGTGCACTGA |
| mir-148b | ACAAAGTTCTGTGATGCACTGAACAAAGTTCTGTGATGCACTGAACAAAGTTCTGTGATGCACTGA |
| miR-149 | GGAGTGAAGACACGGAGCCAGAGGAGTGAAGACACGGAGCCAGAGGAGTGAAGACACGGAGCCAGA |
| miR-150 | ACACTGGTACAAGGGTTGGGAGAACACTGGTACAAGGGTTGGGAGAACACTGGTACAAGGGTTGGGAGA |
| miR-150 | GCACTGGTACAAGGGTTGGGAGAGCACTGGTACAAGGGTTGGGAGAGCACTGGTACAAGGGTTGGGAGA |
| (mouse) | |
| miR-151 | ACCTCAAGGAGCCTCAGTCTAGACCTCAAGGAGCCTCAGTCTAGACCTCAAGGAGCCTCAGTCTAG |
| miR-152 | CCAAGTTCTGTCATGCACTGACCAAGTTCTGTCATGCACTGACCAAGTTCTGTCATGCACTGA |
| miR-153 | TCACTTTTGTGACTATGCAATCACTTTTGTGACTATGCAATCACTTTTGTGACTATGCAA |
| miR-154 | CGAAGGCAACACGGATAACCTACGAAGGCAACACGGATAACCTACGAAGGCAACACGGATAACCTA |
| miR-155 | CCCCTATCACAATTAGCATTAACCCCTATCACAATTAGCATTAACCCCTATCACAATTAGCATTAA |
| [BIC-RNA] | |
| mir-172 | AACAACCAGCTAAGACACTGCCAAACAACCAGCTAAGACACTGCCAAACAACCAGCTAAGACACTGCCA |
| miR-181 | ACTCACCGACAGCGTTGAATGTTACTCACCGACAGCGTTGAATGTTACTCACCGACAGCGTTGAATGTT |
| mir-181b | AACCCACCGACAGCAATGAATGTTAACCCACCGACAGCAATGAATGTTAACCCACCGACAGCAATGAATGTT |
| mir-181c | ACTCACCGACAGGTTGAATGTTACTCACCGACAGGTTGAATGTTACTCACCGACAGGTTGAATGTT |
| miR-182 | TGTGAGTTCTACCATTGCCAAATGTGAGTTCTACCATTGCCAAATGTGAGTTCTACCATTGCCAAA |
| miR-183 | CAGTGAATTCTACCAGTGCCATACAGTGAATTCTACCAGTGCCATACAGTGAATTCTACCAGTGCCATA |
| miR-184 | ACCCTTATCAGTTCTCCGTCCAACCCTTATCAGTTCTCCGTCCAACCCTTATCAGTTCTCCGTCCA |
| miR-185 | GAACTGCCTTTCTCTCCAGAACTGCCTTTCTCTCCAGAACTGCCTTTCTCTCCA |
| miR-186 | AAGCCCAAAAGGAGAATTCTTTGAAGCCCAAAAGGAGAATTCTTTGAAGCCCAAAAGGAGAATTCTTTG |
| miR-187 | CCGGCTGCAACACAAGACACGACCGGCTGCAACACAAGACACGACCGGCTGCAACACAAGACACGA |
| miR-188 | ACCCTCCACCATGCAAGGGATGACCCTCCACCATGCAAGGGATGACCCTCCACCATGCAAGGGATG |
| miR-189 | ACTGATATCAGCTCAGTAGGCACACTGATATCAGCTCAGTAGGCACACTGATATCAGCTCAGTAGGCAC |
| miR-190 | ACCTAATATATCAAACATATCAACCTAATATATCAAACATATCAACCTAATATATCAAACATATCA |
| miR-191 | AGCTGCTTTTGGGATTCCGTTGAGCTGCTTTTGGGATTCCGTTGAGCTGCTTTTGGGATTCCGTTG |
| miR-192 | GGCTGTCAATTCATAGGTCAGGGCTGTCAATTCATAGGTCAGGGCTGTCAATTCATAGGTCAG |
| miR-193 | CTGGGACTTTGTAGGCCAGTTCTGGGACTTTGTAGGCCAGTTCTGGGACTTTGTAGGCCAGTT |
| miR-194 | TCCACATGGAGTTGCTGTTACATCCACATGGAGTTGCTGTTACATCCACATGGAGTTGCTGTTACA |
| miR-195 | GCCAATATTTCTGTGCTGCTAGCCAATATTTCTGTGCTGCTAGCCAATATTTCTGTGCTGCTA |
| miR-196 | cCCAACAACATGAAACTACCTAcCCAACAACATGAAACTACCTAcCCAACAACATGAAACTACCTA |
| miR-197 | GCTGGGTGGAGAAGGTGGTGAAGCTGGGTGGAGAAGGTGGTGAAGCTGGGTGGAGAAGGTGGTGAA |
| miR-198 | CCTATCTCCCCTCTGGACCCCTATCTCCCCTCTGGACCCCTATCTCCCCTCTGGACC |
| miR-199-s | GAACAGGTAGTCTGAACACTGGGGAACAGGTAGTCTGAACACTGGGGAACAGGTAGTCTGAACACTGGG |
| miR-199-as | AACCAATGTGCAGACTACTGTAAACCAATGTGCAGACTACTGTAAACCAATGTGCAGACTACTGTA |
| miR-199b | GAACAGATAGTCTAAACACTGGGGAACAGATAGTCTAAACACTGGGGAACAGATAGTCTAAACACTGGG |
| miR-200b | GTCATCATTACCAGGCAGTATTAGTCATCATTACCAGGCAGTATTAGTCATCATTACCAGGCAGTATTA |
| miR-201 | AGAACAATGCCTTACTGAGTAAGAACAATGCCTTACTGAGTAAGAACAATGCCTTACTGAGTA |
| miR-202 | TCTTCCCATGCGCTATACCTCTTCTTCCCATGCGCTATACCTCTTCTTCCCATGCGCTATACCTCT |
| miR-203 | TCTAGTGGTCCTAAACATTTCATCTAGTGGTCCTAAACATTTCATCTAGTGGTCCTAAACATTTCA |
| miR-204 | CAGGCATAGGATGACAAAGGGAACAGGCATAGGATGACAAAGGGAACAGGCATAGGATGACAAAGGGAA |
| miR-205 | CAGACTCCGGTGGAATGAAGGACAGACTCCGGTGGAATGAAGGACAGACTCCGGTGGAATGAAGGA |
| miR-206 | CCACACACTTCCTTACATTCCACCACACACTTCCTTACATTCCACCACACACTTCCTTACATTCCA |
| miR-207 | GAGGGAGGAGAGCCAGGAGAAGCGAGGGAGGAGAGCCAGGAGAAGCGAGGGAGGAGAGCCAGGAGAAGC |
| miR-208 | ACAAGCTTTTTGCTCGTCTTATACAAGCTTTTTGCTCGTCTTATACAAGCTTTTTGCTCGTCTTAT |
| mir-210 | CAGCCGCTGTCACACGCACAGCAGCCGCTGTCACACGCACAGCAGCCGCTGTCACACGCACAG |
| mir-211 | AGGCGAAGGATGACAAAGGGAAAGGCGAAGGATGACAAAGGGAAAGGCGAAGGATGACAAAGGGAA |
| miR-212 | GGCCGTGACTGGAGACTGTTAGGCCGTGACTGGAGACTGTTAGGCCGTGACTGGAGACTGTTA |
| mir-213 | GGTACAATCAACGGTCGATGGTGGTACAATCAACGGTCGATGGTGGTACAATCAACGGTCGATGGT |
| mir-214 | CTGCCTGTCTGTGCCTGCTGTCTGCCTGTCTGTGCCTGCTGTCTGCCTGTCTGTGCCTGCTGT |
| mir-215 | GTCTGTCAATTCATAGGTCATGTCTGTCAATTCATAGGTCATGTCTGTCAATTCATAGGTCAT |
| mir-216 | CACAGTTGCCAGCTGAGATTACACAGTTGCCAGCTGAGATTACACAGTTGCCAGCTGAGATTA |
| mir-217 | ATCCAATCAGTTCCTGATGCAGTAATCCAATCAGTTCCTGATGCAGTAATCCAATCAGTTCCTGATGCAGTA |
| mir-217 | ATCCAGTCAGTTCCTGATGCAGTAATCCAGTCAGTTCCTGATGCAGTAATCCAGTCAGTTCCTGATGCAGTA |
| (mouse) | |
| mir-218 | CACATGGTTAGATCAAGCACAACACATGGTTAGATCAAGCACAACACATGGTTAGATCAAGCACAA |
| mir-219 | AGAATTGCGTTTGGACAATCAAGAATTGCGTTTGGACAATCAAGAATTGCGTTTGGACAATCA |
| mir-220 | AAAGTGTCAGATACGGTGTGGAAAGTGTCAGATACGGTGTGGAAAGTGTCAGATACGGTGTGG |
| miR-221 | GAAACCCAGCAGACAATGTAGCTGAAACCCAGCAGACAATGTAGCTGAAACCCAGCAGACAATGTAGCT |
| mir-222 | GAGACCCAGTAGCCAGATGTAGCTGAGACCCAGTAGCCAGATGTAGCTGAGACCCAGTAGCCAGATGTAGCT |
| mir-223 | GGGGTATTTGACAAACTGACAGGGGTATTTGACAAACTGACAGGGGTATTTGACAAACTGACA |
| mir-224 | TAAACGGAACCACTAGTGACTTGTAAACGGAACCACTAGTGACTTGTAAACGGAACCACTAGTGACTTG |
| mir-226 | CCAGCAGCACCTGGGGCAGTCCAGCAGCACCTGGGGCAGTCCAGCAGCACCTGGGGCAGT |
| mir-227 | ACACCAATGCCCTAGGGGATGCGACACCAATGCCCTAGGGGATGCGACACCAATGCCCTAGGGGATGCG |
| mir-229 | ACTGGAGGAAGGGCCCAGAGGACTGGAGGAAGGGCCCAGAGGACTGGAGGAAGGGCCCAGAGG |
| mir-231 | AAGAAAGGCAGCAGGTCGTATAGAAGAAAGGCAGCAGGTCGTATAGAAGAAAGGCAGCAGGTCGTATAG |
| mir-232 | ACGGAAGGGCAGAGAGGGCCAGACGGAAGGGCAGAGAGGGCCAGACGGAAGGGCAGAGAGGGCCAG |
| mir-233 | AAAAAGGTTAGCTGGGTGTGTTAAAAAGGTTAGCTGGGTGTGTTAAAAAGGTTAGCTGGGTGTGTT |
| mir-239 | TGTCCGTGGTTCTACCCTGTGGTATGTCCGTGGTTCTACCCTGTGGTATGTCCGTGGTTCTACCCTGTGGTA |
| mir-240 | ACATTTTTCGTTATTGCTCTTGAACATTTTTCGTTATTGCTCTTGAACATTTTTCGTTATTGCTCTTGA |
| mir-244 | TCAACAAAATCACTGATGCTGGATCAACAAAATCACTGATGCTGGATCAACAAAATCACTGATGCTGGA |
| mir-248 | GACGGGTGCGATTTCTGTGTGAGAGACGGGTGCGATTTCTGTGTGAGAGACGGGTGCGATTTCTGTGTGAGA |
| mir-250 | ACAGTCAGGCTTTGGCTAGATCAACAGTCAGGCTTTGGCTAGATCAACAGTCAGGCTTTGGCTAGATCA |
| mir-251 | GCACTGGACTAGGGGTCAGCAGCACTGGACTAGGGGTCAGCAGCACTGGACTAGGGGTCAGCA |
| mir-258 | ATGCTTTTTGGGGTAAGGGCTTATGCTTTTTGGGGTAAGGGCTTATGCTTTTTGGGGTAAGGGCTT |
| mir-290 | AAAAAGTGCCCCCATAGTTTGAGAAAAAGTGCCCCCATAGTTTGAGAAAAAGTGCCCCCATAGTTTGAG |
| mir-291-s | AGAGAGGGCCTCCACTTTGATGAGAGAGGGCCTCCACTTTGATGAGAGAGGGCCTCCACTTTGATG |
| mir-291-as | GGCACACAAAGTGGAAGCACTTTGGCACACAAAGTGGAAGCACTTTGGCACACAAAGTGGAAGCACTTT |
| mir-292-s | CAAAAGAGCCCCCAGTTTGAGTCAAAAGAGCCCCCAGTTTGAGTCAAAAGAGCCCCCAGTTTGAGT |
| mir-292-as | ACACTCAAAACCTGGCGGCACTTACACTCAAAACCTGGCGGCACTTACACTCAAAACCTGGCGGCACTT |
| mir-293 | ACACTACAAACTCTGCGGCACTACACTACAAACTCTGCGGCACTACACTACAAACTCTGCGGCACT |
| mir-294 | ACACACAAAAGGGAAGCACTTTACACACAAAAGGGAAGCACTTTACACACAAAAGGGAAGCACTTT |
| mir-295 | AGACTCAAAAGTAGTAGCACTTTAGACTCAAAAGTAGTAGCACTTTAGACTCAAAAGTAGTAGCACTTT |
| mir-296 | ACAGGATTGAGGGGGGGCCCTACAGGATTGAGGGGGGGCCCTACAGGATTGAGGGGGGGCCCT |
| mir-297 | CATGCACATGCACACATACATCATGCACATGCACACATACATCATGCACATGCACACATACAT |
| mir-298 | GGAAGAACAGCCCTCCTCTGCCGGAAGAACAGCCCTCCTCTGCCGGAAGAACAGCCCTCCTCTGCC |
| mir-299 | ATGTATGTGGGACGGTAAACCAATGTATGTGGGACGGTAAACCAATGTATGTGGGACGGTAAACCA |
| mir-300 | GAAGAGAGCTTGCCCTTGCATAGAAGAGAGCTTGCCCTTGCATAGAAGAGAGCTTGCCCTTGCATA |
| mir-301 | GCTTTGACAATACTATTGCACTGGCTTTGACAATACTATTGCACTGGCTTTGACAATACTATTGCACTG |
| mir-302 | TCACCAAAACATGGAAGCACTTATCACCAAAACATGGAAGCACTTATCACCAAAACATGGAAGCACTTA |
| mir-320 | TTCGCCCTCTCAACCCAGCTTTTTTCGCCCTCTCAACCCAGCTTTTTTCGCCCTCTCAACCCAGCTTTT |
| mir-321 | GAACCCACAATCCCTGGCTTAGAACCCACAATCCCTGGCTTAGAACCCACAATCCCTGGCTTA |
| mir-322 | TGTTGCAGCGCTTCATGTTTTGTTGCAGCGCTTCATGTTTTGTTGCAGCGCTTCATGTTT |
| (rat) | |
| mir-323 | AGAGGTCGACCGTGTAATGTGCAGAGGTCGACCGTGTAATGTGCAGAGGTCGACCGTGTAATGTGC |
| (rat) | |
| mir-324-3p | CCAGCAGCACCTGGGGCAGTGGCCAGCAGCACCTGGGGCAGTGGCCAGCAGCACCTGGGGCAGTGG |
| (rat) | |
| mir-324-5p | ACACCAATGCCCTAGGGGATGCGACACCAATGCCCTAGGGGATGCGACACCAATGCCCTAGGGGATGCG |
| (rat) | |
| mir-325 | ACACTTACTGAGCACCTACTAGGACACTTACTGAGCACCTACTAGGACACTTACTGAGCACCTACTAGG |
| (rat) | |
| mir-326 | ACTGGAGGAAGGGCCCAGAGGACTGGAGGAAGGGCCCAGAGGACTGGAGGAAGGGCCCAGAGG |
| (rat) | |
| mir-327 | ACCCTCATGCCCCTCAAGGACCCTCATGCCCCTCAAGGACCCTCATGCCCCTCAAGG |
| (rat) | |
| mir-328 | ACGGAAGGGCAGAGAGGGCCAGACGGAAGGGCAGAGAGGGCCAGACGGAAGGGCAGAGAGGGCCAG |
| (rat) | |
| mir-329 | AAAAAGGTTAGCTGGGTGTGTTAAAAAGGTTAGCTGGGTGTGTTAAAAAGGTTAGCTGGGTGTGTT |
| (rat) | |
| mir-330 | TTCTCTGCAGGCCCTGTGCTTTGCTTCTCTGCAGGCCCTGTGCTTTGCTTCTCTGCAGGCCCTGTGCTTTGC |
| (rat) | |
| mir-331 | TTCTAGGATAGGCCCAGGGGCTTCTAGGATAGGCCCAGGGGCTTCTAGGATAGGCCCAGGGGC |
| (rat) | |
| mir-332 | TGGCGACTCTGGTGGGACCTGGCGACTCTGGTGGGACCTGGCGACTCTGGTGGGACC |
| (rat) | |
| mir-333 | AAAAGTAACTAGCACACCACAAAAGTAACTAGCACACCACAAAAGTAACTAGCACACCAC |
| (rat) | |
| mir-334 | CACATCTCTGCACCGTTTACACATCTCTGCACCGTTTACACATCTCTGCACCGTTTA |
| (rat) | |
| mir-335 | ACATTTTTCGTTATTGCTCTTGAACATTTTTCGTTATTGCTCTTGAACATTTTTCGTTATTGCTCTTGA |
| (rat) | |
| mir-336 | AGACTAGATATGGAAGGGTGAAGACTAGATATGGAAGGGTGAAGACTAGATATGGAAGGGTGA |
| (rat) | |
| mir-337 | AAAGGCATCATATAGGAGCTGAAAAAGGCATCATATAGGAGCTGAAAAAGGCATCATATAGGAGCTGAA |
| (rat) | |
| mir-338 | TCAACAAAATCACTGATGCTGGATCAACAAAATCACTGATGCTGGATCAACAAAATCACTGATGCTGGA |
| (rat) | |
| mir-339 | AATGAGCTCCTGGAGGACAGGGAAATGAGCTCCTGGAGGACAGGGAAATGAGCTCCTGGAGGACAGGGA |
| (rat) | |
| mir-340 | GGCTATAAAGTAACTGAGACGGAGGCTATAAAGTAACTGAGACGGAGGCTATAAAGTAACTGAGACGGA |
| (rat) | |
| mir-341 | ACTGACCGACCGACCGATCGAACTGACCGACCGACCGATCGAACTGACCGACCGACCGATCGA |
| (rat) | |
| mir-342 | GACGGGTGCGATTTCTGTGTGAGAGACGGGTGCGATTTCTGTGTGAGAGACGGGTGCGATTTCTGTGTGAGA |
| (rat) | |
| mir-343 | AACTGGGCACACGGAGGGAGAAACTGGGCACACGGAGGGAGAAACTGGGCACACGGAGGGAGA |
| (rat) | |
| mir-344 | ACGGTCAGGCTTTGGCTAGATCAACGGTCAGGCTTTGGCTAGATCAACGGTCAGGCTTTGGCTAGATCA |
| (rat) | |
| mir-345 | GCACTGGACTAGGGGTCAGCAGCACTGGACTAGGGGTCAGCAGCACTGGACTAGGGGTCAGCA |
| (rat) | |
| mir-346 | TTAGAGGCAGGCACTCAGGCAGACATTAGAGGCAGGCACTCAGGCAGACATTAGAGGCAGGCACTCAGGCAGACA |
| (rat) | |
| mir-347 | TGGCGACCCAGAGGGACATGGCGACCCAGAGGGACATGGCGACCCAGAGGGACA |
| (rat) | |
| miR-348 | ACTGGAGTGGGGTAAAGGGTGGGCAACTGGAGTGGGGTAAAGGGTGGGCAACTGGAGTGGGGTAAAGGGTGGGCA |
| (rat) | |
| mir-349 | AGAGGTTAAGACAGCAGGGCTGAGAGGTTAAGACAGCAGGGCTGAGAGGTTAAGACAGCAGGGCTG |
| (rat) | |
| mir-350 | GTGAAAGTGTATGGGCTTTGTGAATGTGAAAGTGTATGGGCTTTGTGAATGTGAAAGTGTATGGGCTTTGTGAAT |
| (rat) | |
| mir-351 | ACAGGCTCAAAGGGCTCCTCAGGGAACAGGCTCAAAGGGCTCCTCAGGGAACAGGCTCAAAGGGCTCCTCAGGGA |
| (rat) | |
| mir-352 | TACTATGCAACCTACTACTCTTACTATGCAACCTACTACTCTTACTATGCAACCTACTACTCT |
| (rat) | |
| tRNA-thr | GACCAGTGCTCTAACCCCTGAGCTAGACCAGTGCTCTAACCCCTGAGCTAGACCAGTGCTCTAACCCCTGAGCTA |

| mismatch | |
|---|---|
| probes | (mismatched nucleotides in lower case) |
| miR-101-mm | TCAcTTATaACAGTAaTGTATCAaTTATaACAGTAaTGTATCAcTTATaACAGTAaTGTA |
| miR-103-mm | TCATAcCCCTGTAaAATGCTcCTTCATAcCCCTGTAaAATGCTcCTTCATAcCCCTGTAaAATGCTcCT |
| miR-117-mm | AAaTGTTaCTGCTcAAAAaTGTTaCTGCTcAAAAaTGTTaCTGCTcAA |
| miR-118-mm | ATCaTACAaTCAAcGCATATCaTACAaTCAAcGCATATCaTACAaTCAAcGCAT |
| miR-128-mm | AAAAcAGACCcGTTCAaTGTGAAAAAcAGACCcGTTCAaTGTGAAAAAcAGACCcGTTCAaTGTGA |
| miR-129-mm | GCAAcCCCAGAaCcAAAAAAGGCAAcCCCAGAaCcAAAAAAGGCAAcCCCAGAaCcAAAAAAG |
| miR-139-mm | AGAaACGTcCAaTGTAGAAGAaACGTcCAaTGTAGAAGAaACGTcCAaTGTAGA |
| miR-140s-mm | CTACaATAGcGTAAAAaCACTCTACaATAGcGTAAAAaCACTCTACaATAGcGTAAAAaCACT |
| miR-149-mm | GGAcTGAAGAaACGGAGaCAGAGGAcTGAAGAaACGGAGaCAGAGGAcTGAAGAaACGGAGaCAGA |
| miR-150-mm | ACAaTGGTACAAcGGTTGGcAGAACAaTGGTACAAcGGTTGGcAGAACAaTGGTACAAcGGTTGGcAGA |
| miR-184-mm | ACCaTTATCAcTTCTCCGTaCAACCaTTATCAcTTCTCCGTaCAACCaTTATCAcTTCTCCGTaCA |
| miR-185-mm | GAACTcCCTTTaTCTaCAGAACTcCCTTTaTCTaCAGAACTcCCTTTaTCTaCA |
| miR-198-mm | CCTATaTCCCaTCTGcACCCCTATaTCCCaTCTGcACCCCTATaTCCCaTCTGcACC |
| miR-199-s-mm | GAAaAGGTAcTCTGAACAaTGGGGAAaAGGTAcTCTGAACAaTGGGGAAaAGGTAcTCTGAACAaTGGG |
| mir-211-mm | AGGaGAAGcATGACAAAcGGAAAGGaGAAGcATGACAAAcGGAAAGGaGAAGcATGACAAAcGGAA |
| mir-212-mm | GGCaGTGACTcGAGAaTGTTAGGCaGTGACTcGAGAaTGTTAGGCaGTGACTcGAGAaTGTTA |
| mir-224-mm | TAAAaGGAACaACTAGTcACTTGTAAAaGGAACaACTAGTcACTTGTAAAaGGAACaACTAGTcACTTG |
| mir-258-mm | ATGaTTTTTGGcGTAAGcGCTTATGaTTTTTGGcGTAAGcGCTTATGaTTTTTGGcGTAAGcGCTT |
| mir-290-mm | AAAAAcTGCCaCCATAcTTTGAGAAAAAcTGCCaCCATAcTTTGAGAAAAAcTGCCaCCATAcTTTGAG |
| mir-301-mm | GCTTTcACAATAaTATTGaACTGGCTTTcACAATAaTATTGaACTGGCTTTcACAATAaTATTGaACTG |
| mir-302-mm | TCACaAAAACATcGAAGCAaTTATCACaAAAACATcGAAGCAaTTATCACaAAAACATcGAAGCAaTTA |
| mir-331-mm | TTaTAGGATAcGCCCAGcGGCTTaTAGGATAcGCCCAGcGGCTTaTAGGATAcGCCCAGcGGC |
| mir-332-mm | TGGaGACTaTGGTGGcACCTGGaGACTaTGGTGGcACCTGGaGACTaTGGTGGcACC |
| mir-345-mm | GCAaTGGAaTAGGGcTCAGCAGCAaTGGAaTAGGGcTCAGCAGCAaTGGAaTAGGGcTCAGCA |
| mir-346-mm | TTAGAcGCAGGaACTCAGGaAGACATTAGAcGCAGGaACTCAGGaAGACATTAGAcGCAGGaACTCAGGaAGACA |
| tRNA-thr-mm | GACaAGTGCTCTAAaCCCTcAGCTAGACaAGTGCTCTAAaCCCTcAGCTAGACaAGTGCTCTAAaCCCTcAGCTA |

10 μg of total RNA, isolated with Trizol Reagent (Invitrogen), was spun through a Microcon YM-100 column (Amicon) to enrich for low molecular weight RNA and end-labelled with t ³²P-ATP using the KinaseMax kit (Ambion). After removing unincorporated radionucleotide with Microspin G-25 columns (Amersham), labelled RNA was hybridized to membranes with MicroHyb buffer (Invitrogen). Signals were quantified using a Personal FX phosphoimager (Bio-Rad).

### Northern Blot Analysis

For miRNA northerns, 20 μg of total RNA was separated on 15% denaturing polyacrylamide gels, electrotransferred to GeneScreen Plus membranes, and hybridized with UltraHyb-Oligo buffer (Ambion). Oligonucleotides complementary to mature miRNAs, end-labelled with T4 Kinase (Invitrogen), were used as probes. Probe sequences were as follows:

| | |
|---|---|
| miR-17-5p, | 5′-ACTACCTGCACTGTAAGCACTTTG-3′; |
| miR-18a, | 5′-TATCTGCACTAGATGCACCTTA-3′; |
| miR-19a, | 5′-TCAGTTTTGCATAGATTTGCACA-3′; |
| miR-20a, | 5′-CTACCTGCACTATAAGCACTTTA-3′; |
| miR-92, | 5′-ACAGGCCGGGACAAGTGCAATA-3′, |
| miR-30c, | 5′-GCTGAGAGTGTAGGATGTTTACA-3′; |
| miR-16, | 5′-CGCCAATATTTACGTGCTGCTA-3′. |

For conventional northern blotting, 20 μg of total RNA was separated on 1.2% formaldehyde-agarose gels, transferred to GeneScreen Plus, and hybridized in Ultrahyb buffer (Ambion) with randomly-primed labelled probes. Probes were generated by PCR with the following primers:

miR-17 cluster probe,

| | |
|---|---|
| sense | 5′-ACATGGACTAAATTGCCTTTAAATG-3′, |
| antisense | 5′-AATCTTCAGTTTTACAAGGTGATG-3′; |

miR-106a cluster probe,

| | |
|---|---|
| sense | 5′-CATCCTGGGTTTTACATGCTCC-3′, |
| antisense | 5′-CAAAATTTTAAGTCTTCCAGGAGC-3′; |

7SK RNA probe,

| | |
|---|---|
| sense | 5′-GACATCTGTCACCCCATTGATC-3′, |
| antisense | 5′-TCTGCAGTCTTGGAAGCTTGAC-3′, |

E2F1 probe,

| | |
|---|---|
| sense | 5′-TGTGTGCATGAGTCCATGTGTG-3′, |
| antisense | 5′-GCAAATCAAAGTGCAGATTGGAG-3′. |

### Western Blot Analysis

Antibodies for immunoblotting were as follows: anti-c-Myc mouse monoclonal clone 9E10 (Zymed), anti-E2F1 mouse monoclonal clones KH20 and KH95 (Upstate), anti-α-tubulin mouse monoclonal (Calbiochem). Scanned images were quantified using Quantity One software (Bio-Rad).

Chromatin Immunoprecipitation and Real-Time PCR

Cells were cross-linked with formaldehyde and chromatin was immunoprecipitated as previously described (Boyd et al., Proc Natl Acad Sci USA 95, 13887-92 (1998)).

Rabbit polyclonal c-Myc (sc-764, Santa Cruz Biotechnology) and human hepatocyte growth factor (HGF) antibody (sc-7949, Santa Cruz) were used to immunoprecipitate chromatin fragments. Real-time PCR was performed on an ABI 7700 Sequence Detection System with the SYBR Green PCR core reagent kit (Perkin Elmer Applied Biosystems). Sequences of primers used to amplify ChIP samples are provided in Table 2.

TABLE 2

| | | |
|---|---|---|
| Amplicon | Forward Primer | Reverse Primer |

| | | |
|---|---|---|
| 1 | AAACGTTCTGAATGTTCTGGATTGT | CACAGCCTTCTCAAGTCAGCTAAA |
| 2 | ACCTCGGAAACCCACCAAG | TCTCCCTGGGACTCGACG |
| 3 | AAAGGCAGGCTCGTCGTTG | CGGGATAAAGAGTTGTTTCTCCAA |
| 4 | CTCGACTCTTACTCTCACAAATGG | GCTACTGGTGCAGTTAGGTCC |
| 5 | TTTAAACAGGATATTTACGTTCTGC | GAGGAAATCTTCACATCCACG |
| 6 | CCAAGCTGAAGTACAGGCAAACT | TGGGTGGTCTAACCTAGTGTTATGG |
| 7 | TTGTCACTACAGATGGTCTAAAGGTTACTT | TCCTTGTCTCCACTTCCCCA |
| B23 | GCTACATCCGGGACTCACC | GCTGCCATCACAGTACATGC |

For quantitation of the C13orf25 transcript by real-time PCR, amplicon 5 primers were used. Reactions lacking reverse-transcriptase were performed in parallel to ensure that amplified fragments were derived from cDNA.

### 2′-O-methyl Oligoribonucleotides, Sensor Plasmid Construction, and Luciferase Assays

Oligoribonucleotides (scramble, 5′-AAAACCUUUUGACCGAGCGUGUU-3′; miR-17-5p AS, 5′-ACUACCUGCACUGUAAGCACUUUG-3′; miR-20a AS, 5′-CUACCUGCACUAUAAGCACUUUA-3′) were synthesized by Integrated DNA Technologies. Sensor and control luciferase constructs were made by ligating oligonucleotides containing two sites with perfect complementarity to miR-17-5p or miR-20a into the XbaI site of pGL3-control (Promega). Twenty-four hours prior to transfection, HeLa cells were plated at 150,000 cells per well of a 24-well plate. 200 ng sensor or control plasmid plus 80 ng phRL-SV40 (Promega) were transfected alone or in combination with 20 or 40 pmol 2′-O-methyl oligoribonucleotides using Lipofectamine 2000 (Invitrogen). Luciferase assays were performed 24 hours after transfection using the Dual Luciferase Reporter Assay System (Promega). Firefly luciferase activity was normalized to renilla luciferase activity for each transfected well. 2 independent plasmid preps were each transfected at least three times (on different days). Each transfected well was assayed in triplicate.

For analysis of E2F1 mRNA and protein levels, 200 pmol 2′-O-methyl oligoribonucleotides were transfected into HeLa cells growing in 6-well dishes (plated at 170,000 cells per well 24 hours prior to transfection) using oligofectamine (Invitrogen). RNA and protein was harvested 72 hours after transfection.

Overexpression of the miR-17 Cluster

The miR-17 cluster was amplified from genomic DNA and cloned into pcDNA3.1/V5-His-TOPO (Invitrogen). The following primers were used: sense 5′-CTAAATGGACCTCATATCTTTGAG-3′, antisense 5′-GAAAACAAGACAAGATGTATTTACAC-3′. The correct sequence of the amplified product was confirmed by sequencing. The expression plasmid was transfected into HeLa cells using HeLa Monster (Mirus).

### Construction of E2F1 and PTEN Luciferase Reporter Plasmids

Luciferase reporter constructs containing portions of the E2F1 and PTEN 3′ UTRs were generated by amplifying the 3′ UTR segments from HeLa cDNA. XbaI sites were incorporated into primer sequences and XbaI-digested PCR products were ligated into the XbaI site of pGL3-control. For the mutant constructs, primers were used that introduced the desired mutations during PCR. Primer sequences were as follows (positions of mutations in lower case):

PTEN-wt sense
5′-GGCTAGTCTAGAGGCTAAAGAGCTTTGTGATATAC-3′,
PTEN-wt antisense
5′-GGCTAGTCTAGAAAAAAATGTGCAAAACTGCAAAATTC-3′,
PTEN-mut sense
5′-GGCTAGTCTAGAGGCTAAAGAGCTTTGTGATATACTGGTTCACATCC
TACCCCTgTtCtCTTGTGGCAACAG-3′,
PTEN-mut antisense
5′-GGCTAGTCTAGAAAAAAATGaGaAcAACTGCAAAATTCATTGTAATA
GAATGTG-3′;
E2F1-wt sense
5′-GGCTAGTCTAGATGTGTGCATGAGTCCATGTGTG-3′,
E2F1-wt antisense
5′-GGCTAGTCTAGAGCAAATCAAAGTGCAGATTGGAG-3′,
E2F1-mut sense
5′-GGCTAGTCTAGATGTGTGCATGAGTCCATGTGTGCGCGTGGGGGGGC
TCTAACTGgAgTgTCGGCCCTTTTGCTC-3′,
E2F1-mut antisense
5′-GGCTAGTCTAGAGCAAATCAcAcTcCAGATTGGAGGGTGGGGCA
G-3′.

To make these plasmids, the following sequences were cloned into the XbaI site of the plasmid pGL3-control (made by Promega):

E2F1 WT:
TGTGTGCATGAGTCCATGTGTGCGCGTGGGGGGGCTCTAACTGCACTTTC
GGCCCTTTTGCTCTGGGGGTCCCACAAGGCCCAGGGCAGTGCCTGCTCCC
AGAATCTGGTGCTCTGACCAGGCCAGGTGGGGAGGCTTTGGCTGGCTGGG
CGTGTAGGACGGTGAGAGCACTTCTGTCTTAAAGGTTTTTTCTGATTGAA
GCTTTAATGGAGCGTTATTTATTTATCGAGGCCTCTTTGGTGAGCCTGGG
GAATCAGCAAAGGGGAGGAGGGGTGTGGGGTTGATACCCCAACTCCCTCT
ACCCTTGAGCAAGGGCAGGGGTCCCTGAGCTGTTCTTCTGCCCCATACTG
AAGGAACTGAGGCCTGGGTGATTTATTTATTGGGAAAGTGAGGGAGGGAG
ACAGACTGACTGACAGCCATGGGTGGTCAGATGGTGGGGTGGGCCCTCTC
CAGGGGGCCAGTTCAGGGCCCCAGCTGCCCCCCAGGATGGATATGAGATG
GGAGAGGTGAGTGGGGGACCTTCACTGATGTGGGCAGGAGGGGTGGTGAA
GGCCTCCCCCAGCCCAGACCCTGTGGTCCCTCCTGCAGTGTCTGAAGCGC
CTGCCTCCCCACTGCTCTGCCCCACCCTCCAATCTGCACTTTGATTTGC
E2F1 Mut:
TGTGTGCATGAGTCCATGTGTGCGCGTGGGGGGGCTCTAACTGgAgTgTC
GGCCCTTTTGCTCTGGGGGTCCCACAAGGCCCAGGGCAGTGCCTGCTCCC
AGAATCTGGTGCTCTGACCAGGCCAGGTGGGGAGGCTTTGGCTGGCTGGG
CGTGTAGGACGGTGAGAGCACTTCTGTCTTAAAGGTTTTTTCTGATTGAA
GCTTTAATGGAGCGTTATTTATTTATCGAGGCCTCTTTGGTGAGCCTGGG
GAATCAGCAAAGGGGAGGAGGGGTGTGGGGTTGATACCCCAACTCCCTCT
ACCCTTGAGCAAGGGCAGGGGTCCCTGAGCTGTTCTTCTGCCCCATACTG
AAGGAACTGAGGCCTGGGTGATTTATTTATTGGGAAAGTGAGGGAGGGAG
ACAGACTGACTGACAGCCATGGGTGGTCAGATGGTGGGGTGGGCCCTCTC
CAGGGGGCCAGTTCAGGGCCCCAGCTGCCCCCCAGGATGGATATGAGATG
GGAGAGGTGAGTGGGGGACCTTCACTGATGTGGGCAGGAGGGGTGGTGAA
GGCCTCCCCCAGCCCAGACCCTGTGGTCCCTCCTGCAGTGTCTGAAGCGC
CTGCCTCCCCACTGCTCTGCCCCACCCTCCAATCTGgAgTgTGATTTGC

### Luciferase Assays

Twenty-four hours prior to transfection, HeLa cells were plated at 150,000 cells per well of a 24-well plate. 100 ng pGL3-E2F1 or PTEN 3′ UTR construct plus 80 ng phRL-SV40 (Promega) were transfected using Lipofectamine 2000 (Invitrogen). For the E2F1 construct, 3 independent plasmid preps were used and for the PTEN construct, 2 independent plasmid preps were used (each transfected independently at least twice and assayed multiple times for a total number of 10-12 data points). Luciferase assays were performed 24 hours after transfection using the Dual Luciferase Reporter Assay System (Promega). Firefly luciferase activity was normalized to renilla luciferase activity for each transfected well.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An inhibitory nucleic acid molecule that is complementary to a microRNA encoded by the miR-17 cluster, wherein the inhibitory nucleic acid molecule decreases the expression of the microRNA in a cell.

2. The inhibitory nucleic acid molecule of claim 1, wherein the microRNA is selected from the group consisting of mir-17-5p, mir-18a, mir-19a, mir-20a, mir-19b-1, and mir-92-1.

3. The inhibitory nucleic acid molecule of claim 1, wherein the nucleic acid molecule is an antisense nucleic acid molecule.

4. The inhibitory nucleic acid molecule of claim 3, wherein the microRNA is mir-17-5p or mir-20a.

5. The inhibitory nucleic acid molecule of claim 4, wherein the antisense nucleic acid molecule has at least 85% sequence identity to the following nucleic acid sequences:

| | |
|---|---|
| miR-17-5p AS, | |
| 5′-ACUACCUGCACUGUAAGCACUUUG-3′; | (SEQ ID NO: 1) |
| or | |
| miR-20a AS, | |
| 5′-CUACCUGCACUAUAAGCACUUUA-3′. | (SEQ ID NO: 2) |

6. An inhibitory nucleic acid molecule that corresponds to a microRNA encoded by the miR-17 cluster, wherein the inhibitory nucleic acid molecule decreases the expression of the microRNA in a cell, and wherein the inhibitory nucleic acid molecule is an shRNA or an siRNA.

7-8. (canceled)

9. An antisense nucleic acid molecule that is complementary to a mir-17-Sp or mir-20a nucleic acid molecule and comprises a phosphorothioate backbone and a 2′-OMe sugar modification.

10. The antisense nucleic acid molecule of claim 9, wherein the antisense nucleic acid molecule is conjugated to cholesterol.

11. An expression vector encoding an inhibitory nucleic acid molecule of claim 1.

12-13. (canceled)

14. A cell comprising the vector of claim 11 or an inhibitory nucleic acid molecule of claim 1.

15. (canceled)

16. A vector comprising a nucleic acid sequence encoding a reporter gene, wherein the vector further comprises a nucleic acid sequence complementary to a microRNA selected from the group consisting of mir-7-5p, mir-8a, mir-19a, mir-20a, mir-19b-1, and mir-92-1, wherein the complementary sequence is positioned to regulate expression of the reporter gene.

17. (canceled)

18. A vector comprising a nucleic acid sequence encoding a reporter gene, wherein the vector further comprises a 3, untranslated region of an E2F1 gene positioned to regulate expression of the reporter gene.

19. The vector of claim 18, wherein the 3′untranslated region comprises one of the following nucleic acid sequences:
E2F1 WT:
(SEQ ID NO: 307)
TGTGTGCATGAGTCCATGTGTGCGCGTGGGGGGGCTCTAACTGCACTTTC
GGCCCTTTTGCTCTGGGGGTCCCACAAGGCCCAGGGCAGTGCCTGCTCCC
AGAATCTGGTGCTCTGACCAGGCCAGGTGGGGAGGCTTTGGCTGGCTGGG
CGTGTAGGACGGTGAGAGCACTTCTGTCTTAAAGGTTTTTTCTGATTGAA
GCTTTAATGGAGCGTTATTTATTTATCGAGGCCTCTTTGGTGAGCCTGGG
GAATCAGCAAAGGGGAGGAGGGGTGTGGGGTTGATACCCCAACTCCCTCT
ACCCTTGAGCAAGGGCAGGGGTCCCTGAGCTGTTCTTCTGCCCCATACTG
AAGGAACTGAGGCCTGGGTGATTTATTTATTGGGAAAGTGAGGGAGGGAG
ACAGACTGACTGACAGCCATGGGTGGTCAGATGGTGGGGTGGGCCCTCTC
CAGGGGGCCAGTTCAGGGCCCCAGCTGCCCCCCAGGATGGATATGAGATG
GGAGAGGTGAGTGGGGGACCTTCACTGATGTGGGCAGGAGGGGTGGTGAA
GGCCTCCCCCAGCCCAGACCCTGTGGTCCCTCCTGCAGTGTCTGAAGCGC
CTGCCTCCCCACTGCTCTGCCCCACCCTCCAATCTGCACTTTGATTTGC
E2F1 Mut:
(SEQ ID NO: 308)
TGTGTGCATGAGTCCATGTGTGCGCGTGGGGGGGCTCTAACTGgAgTgTC
GGCCCTTTTGCTCTGGGGGTCCCACAAGGCCCAGGGCAGTGCCTGCTCCC
AGAATCTGGTGCTCTGACCAGGCCAGGTGGGGAGGCTTTGGCTGGCTGGG
CGTGTAGGACGGTGAGAGCACTTCTGTCTTAAAGGTTTTTTCTGATTGAA
GCTTTAATGGAGCGTTATTTATTTATCGAGGCCTCTTTGGTGAGCCTGGG
GAATCAGCAAAGGGGAGGAGGGGTGTGGGGTTGATACCCCAACTCCCTCT
ACCCTTGAGCAAGGGCAGGGGTCCCTGAGCTGTTCTTCTGCCCCATACTG
AAGGAACTGAGGCCTGGGTGATTTATTTATTGGGAAAGTGAGGGAGGGAG
ACAGACTGACTGACAGCCATGGGTGGTCAGATGGTGGGGTGGGCCCTCTC
CAGGGGGCCAGTTCAGGGCCCCAGCTGCCCCCCAGGATGGATATGAGATG
GGAGAGGTGAGTGGGGGACCTTCACTGATGTGGGCAGGAGGGGTGGTGAA
GGCCTCCCCCAGCCCAGACCCTGTGGTCCCTCCTGCAGTGTCTGAAGCGC
CTGCCTCCCCACTGCTCTGCCCCACCCTCCAATCTGgAgTGTGATTTGC.

20. A method of decreasing expression of a microRNA of the mir-17 cluster in a cell, the method comprising contacting the cell with an effective amount of an inhibitory nucleic acid molecule complementary to at least a portion of a microRNA nucleic acid molecule selected from the group consisting of mir-17-5p, mir-18a, mir-19a, mir-20a, mir-19b-1, and mir-92-1, wherein the inhibitory nucleic acid molecule decreases expression of a microRNA of the mir-17 cluster in the cell.

21-25. (canceled)

26. A method of treating a subject having a neoplasm, the method comprising administering to the subject an effective amount of an inhibitory nucleic acid molecule complementary to a microRNA of the mir-17 cluster, wherein the inhibitory nucleic acid molecule reduces expression of at least one microRNA selected from the group consisting of mir-17-5p, mir-18a, mir-19a, mir-20a, mir-11b-1, and mir-92-1 thereby treating the neoplasm.

27. (canceled)

28. The method of claim 26 wherein an effective amount of two inhibitory nucleic acid molecules each of which is complementary to a different microRNA of the mir-17 cluster are administered to the subject simultaneously or within 14 days of each other in amounts sufficient to treat a neoplasm.

29-33. (canceled)

34. A method of identifying an agent that treats or prevents a neoplasm, the method comprising
(a) contacting a cell that expresses a microRNA of the mir-17 cluster with an agent, and
(b) comparing the level of microRNA expression in the cell contacted by the agent with the level of expression in a control cell, wherein an agent that decreases microRNA expression thereby treats or prevents a neoplasm.

35. (canceled)

36. A method for diagnosing a subject as having or having a propensity to develop a neoplasia, the method comprising
(a) measuring the level of a marker selected from the group consisting of mir-17-5p, mir-18a, mir-19a, mir-20a, mir-9b-1, and mir-92-1, c-Myc, E2F1, and p21 in a biological sample from the subject, and
(b) detecting an alteration in the level of the marker in the sample relative to the level in a control sample, wherein detection of an alteration in the marker level indicates the subject has or has a propensity to develop a neoplasia.

37-60. (canceled)


SEQUENCE LISTING (324 sequences)

SEQ ID NO: 1 - RNA, 24 nt, Artificial Sequence
  Description of Artificial Sequence Synthetic oligonucleotide
acuaccugcacuguaagcacuuug

SEQ ID NO: 2 - RNA, 23 nt, Artificial Sequence
  Description of Artificial Sequence Synthetic oligonucleotide
cuaccugcacuauaagcacuuua

SEQ ID NO: 3 - DNA, 25 bp, Artificial Sequence
  Description of Artificial Sequence Synthetic oligonucleotide
acatggactaaattgcctttaaatg

SEQ ID NO: 4 - DNA, 24 bp, Artificial Sequence
  Description of Artificial Sequence Synthetic oligonucleotide
aatcttcagttttacaaggtgatg

SEQ ID NO: 5 - DNA, 22 bp, Artificial Sequence
  Description of Artificial Sequence Synthetic oligonucleotide
catcctgggttttacatgctcc

SEQ ID NO: 6 - DNA, 24 bp, Artificial Sequence
  Description of Artificial Sequence Synthetic oligonucleotide
caaaattttaagtcttccaggagc

SEQ ID NO: 7 - DNA, 66 bp, Artificial Sequence
  Description of Artificial Sequence Synthetic oligonucleotide
aactatacaacctactacctcaaactatacaacctactacctcaaactatacaacctactacctca

SEQ ID NO: 8 - DNA, 66 bp, Artificial Sequence
  Description of Artificial Sequence Synthetic oligonucleotide
aaccacacaacctactacctcaaaccacacaacctactacctcaaaccacacaacctactacctca

SEQ ID NO: 9 - DNA, 66 bp, Artificial Sequence
  Description of Artificial Sequence Synthetic oligonucleotide
aaccatacaacctactacctcaaaccatacaacctactacctcaaaccatacaacctactacctca

SEQ ID NO: 10 - DNA, 63 bp, Artificial Sequence
  Description of Artificial Sequence Synthetic oligonucleotide
actatgcaacctactacctctactatgcaacctactacctctactatgcaacctactacctct

SEQ ID NO: 11 - DNA, 63 bp, Artificial Sequence
  Description of Artificial Sequence Synthetic oligonucleotide
actatacaacctcctacctcaactatacaacctcctacctcaactatacaacctcctacctca

SEQ ID NO: 12 - DNA, 66 bp, Artificial Sequence
  Description of Artificial Sequence Synthetic oligonucleotide
aactatacaatctactacctcaaactatacaatctactacctcaaactatacaatctactacctca

SEQ ID NO: 13 - DNA, 66 bp, Artificial Sequence
  Description of Artificial Sequence Synthetic oligonucleotide
tactgtacaaactactacctcatactgtacaaactactacctcatactgtacaaactactacctca

SEQ ID NO: 14 - DNA, 66 bp, Artificial Sequence
  Description of Artificial Sequence Synthetic oligonucleotide
aactgtacacactactacctcaaactgtacacactactacctcaaactgtacacactactacctca

SEQ ID NO: 15 - DNA, 57 bp, Artificial Sequence
  Description of Artificial Sequence Synthetic oligonucleotide
agcacaaactactacctcaagcacaaactactacctcaagcacaaactactacctca

SEQ ID NO: 16 - DNA, 66 bp, Artificial Sequence
  Description of Artificial Sequence Synthetic oligonucleotide
ttacatacttctttacattccattacatacttctttacattccattacatacttctttacattcca

SEQ ID NO: 17 - DNA, 66 bp, Artificial Sequence
  Description of Artificial Sequence Synthetic oligonucleotide
gtacatacttctttacattccagtacatacttctttacattccagtacatacttctttacattcca

SEQ ID NO: 18 - DNA, 69 bp, Artificial Sequence
  Description of Artificial Sequence Synthetic oligonucleotide
aatacatacttctttacattccaaatacatacttctttacattccaaatacatacttctttacattcca

SEQ ID NO: 19 - DNA, 69 bp, Artificial Sequence
  Description of Artificial Sequence Synthetic oligonucleotide
acaacaaaatcactagtcttccaacaacaaaatcactagtcttccaacaacaaaatcactagtcttcca

SEQ ID NO: 20 - DNA, 69 bp, Artificial Sequence
  Description of Artificial Sequence Synthetic oligonucleotide
tcatacagctagataaccaaagatcatacagctagataaccaaagatcatacagctagataaccaaaga

SEQ ID NO: 21 - DNA, 69 bp, Artificial Sequence
  Description of Artificial Sequence Synthetic oligonucleotide
cacaaattcggatctacagggtacacaaattcggatctacagggtacacaaattcggatctacagggta

SEQ ID NO: 22 - DNA, 66 bp, Artificial Sequence
  Description of Artificial Sequence Synthetic oligonucleotide
acacaaattcggttctacagggacacaaattcggttctacagggacacaaattcggttctacaggg

SEQ ID NO: 23 - DNA, 66 bp, Artificial Sequence
  Description of Artificial Sequence Synthetic oligonucleotide
cacaaaccattatgtgctgctacacaaaccattatgtgctgctacacaaaccattatgtgctgcta

SEQ ID NO: 24 - DNA, 66 bp, Artificial Sequence
  Description of Artificial Sequence Synthetic oligonucleotide
tgtaaaccatgatgtgctgctatgtaaaccatgatgtgctgctatgtaaaccatgatgtgctgcta

SEQ ID NO: 25 - DNA, 66 bp, Artificial Sequence
  Description of Artificial Sequence Synthetic oligonucleotide
cgccaatatttacgtgctgctacgccaatatttacgtgctgctacgccaatatttacgtgctgcta

SEQ ID NO: 26 - DNA, 60 bp, Artificial Sequence
  Description of Artificial Sequence Synthetic oligonucleotide
acaagtgccttcactgcagtacaagtgccttcactgcagtacaagtgccttcactgcagt

SEQ ID NO: 27 - DNA, 60 bp, Artificial Sequence
  Description of Artificial Sequence Synthetic oligonucleotide
acaagtgccctcactgcagtacaagtgccctcactgcagtacaagtgccctcactgcagt

SEQ ID NO: 28 - DNA, 66 bp, Artificial Sequence
  Description of Artificial Sequence Synthetic oligonucleotide
tatctgcactagatgcaccttatatctgcactagatgcaccttatatctgcactagatgcacctta

SEQ ID NO: 29 - DNA, 69 bp, Artificial Sequence
  Description of Artificial Sequence Synthetic oligonucleotide
tcagttttgcatagatttgcacatcagttttgcatagatttgcacatcagttttgcatagatttgcaca

SEQ ID NO: 30 - DNA, 69 bp, Artificial Sequence
  Description of Artificial Sequence Synthetic oligonucleotide
tcagttttgcatggatttgcacatcagttttgcatggatttgcacatcagttttgcatggatttgcaca

SEQ ID NO: 31 - DNA, 69 bp, Artificial Sequence
  Description of Artificial Sequence Synthetic oligonucleotide
ctacctgcactataagcactttactacctgcactataagcactttactacctgcactataagcacttta

SEQ ID NO: 32 - DNA, 66 bp, Artificial Sequence
  Description of Artificial Sequence Synthetic oligonucleotide
tcaacatcagtctgataagctatcaacatcagtctgataagctatcaacatcagtctgataagcta

SEQ ID NO: 33 - DNA, 66 bp, Artificial Sequence
  Description of Artificial Sequence Synthetic oligonucleotide
acagttcttcaactggcagcttacagttcttcaactggcagcttacagttcttcaactggcagctt

SEQ ID NO: 34 - DNA, 63 bp, Artificial Sequence
  Description of Artificial Sequence Synthetic oligonucleotide
ggaaatccctggcaatgtgatggaaatccctggcaatgtgatggaaatccctggcaatgtgat

SEQ ID NO: 35 - DNA, 69 bp, Artificial Sequence
  Description of Artificial Sequence Synthetic oligonucleotide
gtggtaatccctggcaatgtgatgtggtaatccctggcaatgtgatgtggtaatccctggcaatgtgat

SEQ ID NO: 36 - DNA, 66 bp, Artificial Sequence
  Description of Artificial Sequence Synthetic oligonucleotide
ctgttcctgctgaactgagccactgttcctgctgaactgagccactgttcctgctgaactgagcca

SEQ ID NO: 37 - DNA, 66 bp, Artificial Sequence
  Description of Artificial Sequence Synthetic oligonucleotide
tcagaccgagacaagtgcaatgtcagaccgagacaagtgcaatgtcagaccgagacaagtgcaatg

SEQ ID NO: 38 - DNA, 66 bp, Artificial Sequence
  Description of Artificial Sequence Synthetic oligonucleotide
agcctatcctggattacttgaaagcctatcctggattacttgaaagcctatcctggattacttgaa

SEQ ID NO: 39 - DNA, 66 bp, Artificial Sequence
  Description of Artificial Sequence Synthetic oligonucleotide
aacctatcctgaattacttgaaaacctatcctgaattacttgaaaacctatcctgaattacttgaa

SEQ ID NO: 40 - DNA, 66 bp, Artificial Sequence
  Description of Artificial Sequence Synthetic oligonucleotide
agcggaacttagccactgtgaaagcggaacttagccactgtgaaagcggaacttagccactgtgaa

SEQ ID NO: 41 - DNA, 60 bp, Artificial Sequence
  Description of Artificial Sequence Synthetic oligonucleotide
cagaacttagccactgtgaacagaacttagccactgtgaacagaacttagccactgtgaa

SEQ ID NO: 42 - DNA, 66 bp, Artificial Sequence
  Description of Artificial Sequence Synthetic oligonucleotide
ctcaatagactgtgagctccttctcaatagactgtgagctccttctcaatagactgtgagctcctt

SEQ ID NO: 43 - DNA, 66 bp, Artificial Sequence
  Description of Artificial Sequence Synthetic oligonucleotide
aaccgatttcagatggtgctagaaccgatttcagatggtgctagaaccgatttcagatggtgctag

SEQ ID NO: 44 - DNA, 69 bp, Artificial Sequence
  Description of Artificial Sequence Synthetic oligonucleotide
aacactgatttcaaatggtgctaaacactgatttcaaatggtgctaaacactgatttcaaatggtgcta

SEQ ID NO: 45 - DNA, 69 bp, Artificial Sequence
  Description of Artificial Sequence Synthetic oligonucleotide
taaccgatttcaaatggtgctagtaaccgatttcaaatggtgctagtaaccgatttcaaatggtgctag

SEQ ID NO: 46 - DNA, 69 bp, Artificial Sequence
  Description of Artificial Sequence Synthetic oligonucleotide
gcttccagtcgaggatgtttacagcttccagtcgaggatgtttacagcttccagtcgaggatgtttaca

SEQ ID NO: 47 - DNA, 66 bp, Artificial Sequence
  Description of Artificial Sequence Synthetic oligonucleotide
gctgcaaacatccgactgaaaggctgcaaacatccgactgaaaggctgcaaacatccgactgaaag

SEQ ID NO: 48 - DNA, 63 bp, Artificial Sequence
  Description of Artificial Sequence Synthetic oligonucleotide
gctgagtgtaggatgtttacagctgagtgtaggatgtttacagctgagtgtaggatgtttaca

SEQ ID NO: 49 - DNA, 69 bp, Artificial Sequence
  Description of Artificial Sequence Synthetic oligonucleotide
gctgagagtgtaggatgtttacagctgagagtgtaggatgtttacagctgagagtgtaggatgtttaca

SEQ ID NO: 50 - DNA, 66 bp, Artificial Sequence
  Description of Artificial Sequence Synthetic oligonucleotide
cttccagtcggggatgtttacacttccagtcggggatgtttacacttccagtcggggatgtttaca

SEQ ID NO: 51 - DNA, 60 bp, Artificial Sequence
  Description of Artificial Sequence Synthetic oligonucleotide
tccagtcaaggatgtttacatccagtcaaggatgtttacatccagtcaaggatgtttaca

SEQ ID NO: 52 - DNA, 63 bp, Artificial Sequence
  Description of Artificial Sequence Synthetic oligonucleotide
cagctatgccagcatcttgcccagctatgccagcatcttgcccagctatgccagcatcttgcc

SEQ ID NO: 53 - DNA, 63 bp, Artificial Sequence
  Description of Artificial Sequence Synthetic oligonucleotide
gcaacttagtaatgtgcaatagcaacttagtaatgtgcaatagcaacttagtaatgtgcaata

SEQ ID NO: 54 - DNA, 57 bp, Artificial Sequence
  Description of Artificial Sequence Synthetic oligonucleotide
caatgcaactacaatgcaccaatgcaactacaatgcaccaatgcaactacaatgcac

SEQ ID NO: 55 - DNA, 57 bp, Artificial Sequence
  Description of Artificial Sequence Synthetic oligonucleotide
caatgcaacagcaatgcaccaatgcaacagcaatgcaccaatgcaacagcaatgcac

SEQ ID NO: 56 - DNA, 66 bp, Artificial Sequence
  Description of Artificial Sequence Synthetic oligonucleotide
acaaccagctaagacactgccaacaaccagctaagacactgccaacaaccagctaagacactgcca

SEQ ID NO: 57 - DNA, 72 bp, Artificial Sequence
  Description of Artificial Sequence Synthetic oligonucleotide
actacctgcactgtaagcactttgactacctgcactgtaagcactttgactacctgcactgtaagcactttg

SEQ ID NO: 58 - DNA, 66 bp, Artificial Sequence
  Description of Artificial Sequence Synthetic oligonucleotide
acaggccgggacaagtgcaataacaggccgggacaagtgcaataacaggccgggacaagtgcaata

SEQ ID NO: 59 - DNA, 66 bp, Artificial Sequence
  Description of Artificial Sequence Synthetic oligonucleotide
ctacctgcacgaacagcactttctacctgcacgaacagcactttctacctgcacgaacagcacttt

SEQ ID NO: 60 - DNA, 60 bp, Artificial Sequence
  Description of Artificial Sequence Synthetic oligonucleotide
atctgcactgtcagcactttatctgcactgtcagcactttatctgcactgtcagcacttt

SEQ ID NO: 61 - DNA, 66 bp, Artificial Sequence
  Description of Artificial Sequence Synthetic oligonucleotide
tgctcaataaatacccgttgaatgctcaataaatacccgttgaatgctcaataaatacccgttgaa

SEQ ID NO: 62 - DNA, 66 bp, Artificial Sequence
  Description of Artificial Sequence Synthetic oligonucleotide
gcaaaaatgtgctagtgccaaagcaaaaatgtgctagtgccaaagcaaaaatgtgctagtgccaaa

SEQ ID NO: 63 - DNA, 66 bp, Artificial Sequence
  Description of Artificial Sequence Synthetic oligonucleotide
aacaatacaacttactacctcaaacaatacaacttactacctcaaacaatacaacttactacctca

SEQ ID NO: 64 - DNA, 60 bp, Artificial Sequence
  Description of Artificial Sequence Synthetic oligonucleotide
acaagatcggatctacgggtacaagatcggatctacgggtacaagatcggatctacgggt

SEQ ID NO: 65 - DNA, 66 bp, Artificial Sequence
  Description of Artificial Sequence Synthetic oligonucleotide
cgcaaggtcggttctacgggtgcgcaaggtcggttctacgggtgcgcaaggtcggttctacgggtg

SEQ ID NO: 66 - DNA, 66 bp, Artificial Sequence
  Description of Artificial Sequence Synthetic oligonucleotide
cacaagttcggatctacgggttcacaagttcggatctacgggttcacaagttcggatctacgggtt

SEQ ID NO: 67 - DNA, 60 bp, Artificial Sequence
  Description of Artificial Sequence Synthetic oligonucleotide
tcagttatcacagtactgtatcagttatcacagtactgtatcagttatcacagtactgta

SEQ ID NO: 68 - DNA, 69 bp, Artificial Sequence
  Description of Artificial Sequence Synthetic oligonucleotide
tcatagccctgtacaatgctgcttcatagccctgtacaatgctgcttcatagccctgtacaatgctgct

SEQ ID NO: 69 - DNA, 66 bp, Artificial Sequence
  Description of Artificial Sequence Synthetic oligonucleotide
tagcttatcagactgatgttgatagcttatcagactgatgttgatagcttatcagactgatgttga

SEQ ID NO: 70 - DNA, 60 bp, Artificial Sequence
  Description of Artificial Sequence Synthetic oligonucleotide
acaggagtctgagcatttgaacaggagtctgagcatttgaacaggagtctgagcatttga

SEQ ID NO: 71 - DNA, 72 bp, Artificial Sequence
  Description of Artificial Sequence Synthetic oligonucleotide
gctacctgcactgtaagcacttttgctacctgcactgtaagcacttttgctacctgcactgtaagcactttt

SEQ ID NO: 72 - DNA, 69 bp, Artificial Sequence
  Description of Artificial Sequence Synthetic oligonucleotide
tgatagccctgtacaatgctgcttgatagccctgtacaatgctgcttgatagccctgtacaatgctgct

SEQ ID NO: 73 - DNA, 63 bp, Artificial Sequence
  Description of Artificial Sequence Synthetic oligonucleotide
aatgcccctaaaaatccttataatgcccctaaaaatccttataatgcccctaaaaatccttat

SEQ ID NO: 74 - DNA, 60 bp, Artificial Sequence
  Description of Artificial Sequence Synthetic oligonucleotide
gcgcaggccgactcgaccaggcgcaggccgactcgaccaggcgcaggccgactcgaccag

SEQ ID NO: 75 - DNA, 51 bp, Artificial Sequence
  Description of Artificial Sequence Synthetic oligonucleotide
cgacgtcggccgctcgacgacgtcggccgctcgacgacgtcggccgctcga

SEQ ID NO: 76 - DNA, 57 bp, Artificial Sequence
  Description of Artificial Sequence Synthetic oligonucleotide
ttccgtggatgtcaggaccttccgtggatgtcaggaccttccgtggatgtcaggacc

SEQ ID NO: 77 - DNA, 66 bp, Artificial Sequence
  Description of Artificial Sequence Synthetic oligonucleotide
tggtgggcgcaccagggctcgatggtgggcgcaccagggctcgatggtgggcgcaccagggctcga

SEQ ID NO: 78 - DNA, 66 bp, Artificial Sequence
  Description of Artificial Sequence Synthetic oligonucleotide
cgccaatatttacgtgcagctacgccaatatttacgtgcagctacgccaatatttacgtgcagcta

SEQ ID NO: 79 - DNA, 48 bp, Artificial Sequence
  Description of Artificial Sequence Synthetic oligonucleotide
ttccagctccgcttcattccagctccgcttcattccagctccgcttca

SEQ ID NO: 80 - DNA, 78 bp, Artificial Sequence
  Description of Artificial Sequence Synthetic oligonucleotide
cagcgttcgtcctgagccaggatcaacagcgttcgtcctgagccaggatcaacagcgttcgtcctgagccaggatcaa

SEQ ID NO: 81 - DNA, 48 bp, Artificial Sequence
  Description of Artificial Sequence Synthetic oligonucleotide
aactgttcctgctgaaaactgttcctgctgaaaactgttcctgctgaa

SEQ ID NO: 82 - DNA, 54 bp, Artificial Sequence
  Description of Artificial Sequence Synthetic oligonucleotide
atcctacactcaaggcatatcctacactcaaggcatatcctacactcaaggcat

SEQ ID NO: 83 - DNA, 54 bp, Artificial Sequence
  Description of Artificial Sequence Synthetic oligonucleotide
atggtaatccctggcaatatggtaatccctggcaatatggtaatccctggcaat

SEQ ID NO: 84 - DNA, 51 bp, Artificial Sequence
  Description of Artificial Sequence Synthetic oligonucleotide
acacgctgcgtcccgccacacgctgcgtcccgccacacgctgcgtcccgcc

SEQ ID NO: 85 - DNA, 72 bp, Artificial Sequence
  Description of Artificial Sequence Synthetic oligonucleotide
ttcgggaagggatcaggtggttcattcgggaagggatcaggtggttcattcgggaagggatcaggtggttca

SEQ ID NO: 86 - DNA, 69 bp, Artificial Sequence
  Description of Artificial Sequence Synthetic oligonucleotide
acaaacaccattgtcacactccaacaaacaccattgtcacactccaacaaacaccattgtcacactcca

SEQ ID NO: 87 - DNA, 69 bp, Artificial Sequence
  Description of Artificial Sequence Synthetic oligonucleotide
tcaaacaccattgtcacactccatcaaacaccattgtcacactccatcaaacaccattgtcacactcca

SEQ ID NO: 88 - DNA, 63 bp, Artificial Sequence
  Description of Artificial Sequence Synthetic oligonucleotide
cgcgtaccaaaagtaataatgcgcgtaccaaaagtaataatgcgcgtaccaaaagtaataatg

SEQ ID NO: 89 - DNA, 63 bp, Artificial Sequence
  Description of Artificial Sequence Synthetic oligonucleotide
gcattattactcacggtacgagcattattactcacggtacgagcattattactcacggtacga

SEQ ID NO: 90 - DNA, 66 bp, Artificial Sequence
  Description of Artificial Sequence Synthetic oligonucleotide
tggcattcaccgcgtgccttaatggcattcaccgcgtgccttaatggcattcaccgcgtgccttaa

SEQ ID NO: 91 - DNA, 63 bp, Artificial Sequence
  Description of Artificial Sequence Synthetic oligonucleotide
gcattcacccgcgtgccttaagcattcacccgcgtgccttaagcattcacccgcgtgccttaa

SEQ ID NO: 92 - DNA, 69 bp, Artificial Sequence
  Description of Artificial Sequence Synthetic oligonucleotide
cacaggttaaagggtctcagggacacaggttaaagggtctcagggacacaggttaaagggtctcaggga

SEQ ID NO: 93 - DNA, 66 bp, Artificial Sequence
  Description of Artificial Sequence Synthetic oligonucleotide
tcacaagttagggtctcagggatcacaagttagggtctcagggatcacaagttagggtctcaggga

SEQ ID NO: 94 - DNA, 66 bp, Artificial Sequence
  Description of Artificial Sequence Synthetic oligonucleotide
agccaagctcagacggatccgaagccaagctcagacggatccgaagccaagctcagacggatccga

SEQ ID NO: 95 - DNA, 66 bp, Artificial Sequence
  Description of Artificial Sequence Synthetic oligonucleotide
aaaagagaccggttcactgtgaaaaagagaccggttcactgtgaaaaagagaccggttcactgtga

SEQ ID NO: 96 - DNA, 63 bp, Artificial Sequence
  Description of Artificial Sequence Synthetic oligonucleotide
gcaagcccagaccgaaaaaaggcaagcccagaccgaaaaaaggcaagcccagaccgaaaaaag

SEQ ID NO: 97 - DNA, 66 bp, Artificial Sequence
  Description of Artificial Sequence Synthetic oligonucleotide
agcaagcccagaccgcaaaaagagcaagcccagaccgcaaaaagagcaagcccagaccgcaaaaag

SEQ ID NO: 98 - DNA, 60 bp, Artificial Sequence
  Description of Artificial Sequence Synthetic oligonucleotide
gcccttttaacattgcactggcccttttaacattgcactggcccttttaacattgcactg

SEQ ID NO: 99 - DNA, 60 bp, Artificial Sequence
  Description of Artificial Sequence Synthetic oligonucleotide
gccctttcatcattgcactggccctttcatcattgcactggccctttcatcattgcactg

SEQ ID NO: 100 - DNA, 63 bp, Artificial Sequence
  Description of Artificial Sequence Synthetic oligonucleotide
actttcggttatctagctttaactttcggttatctagctttaactttcggttatctagcttta

SEQ ID NO: 101 - DNA, 69 bp, Artificial Sequence
  Description of Artificial Sequence Synthetic oligonucleotide
gcgaccatggctgtagactgttagcgaccatggctgtagactgttagcgaccatggctgtagactgtta

SEQ ID NO: 102 - DNA, 66 bp, Artificial Sequence
  Description of Artificial Sequence Synthetic oligonucleotide
acagctggttgaaggggaccaaacagctggttgaaggggaccaaacagctggttgaaggggaccaa

SEQ ID NO: 103 - DNA, 66 bp, Artificial Sequence
  Description of Artificial Sequence Synthetic oligonucleotide
tccctctggtcaaccagtcacatccctctggtcaaccagtcacatccctctggtcaaccagtcaca

SEQ ID NO: 104 - DNA, 66 bp, Artificial Sequence
  Description of Artificial Sequence Synthetic oligonucleotide
cccctctggtcaaccagtcacacccctctggtcaaccagtcacacccctctggtcaaccagtcaca

SEQ ID NO: 105 - DNA, 72 bp, Artificial Sequence
  Description of Artificial Sequence Synthetic oligonucleotide
atcacataggaataaaaagccataatcacataggaataaaaagccataatcacataggaataaaaagccata

SEQ ID NO: 106 - DNA, 69 bp, Artificial Sequence
  Description of Artificial Sequence Synthetic oligonucleotide
tccatcatcaaaacaaatggagttccatcatcaaaacaaatggagttccatcatcaaaacaaatggagt

SEQ ID NO: 107 - DNA, 66 bp, Artificial Sequence
  Description of Artificial Sequence Synthetic oligonucleotide
ctacgcgtattcttaagcaatactacgcgtattcttaagcaatactacgcgtattcttaagcaata

SEQ ID NO: 108 - DNA, 51 bp, Artificial Sequence
  Description of Artificial Sequence Synthetic oligonucleotide
gattcacaacaccagctgattcacaacaccagctgattcacaacaccagct

SEQ ID NO: 109 - DNA, 54 bp, Artificial Sequence
  Description of Artificial Sequence Synthetic oligonucleotide
agacacgtgcactgtagaagacacgtgcactgtagaagacacgtgcactgtaga

SEQ ID NO: 110 - DNA, 63 bp, Artificial Sequence
  Description of Artificial Sequence Synthetic oligonucleotide
ctaccatagggtaaaaccactctaccatagggtaaaaccactctaccatagggtaaaaccact

SEQ ID NO: 111 - DNA, 66 bp, Artificial Sequence
  Description of Artificial Sequence Synthetic oligonucleotide
tccgtggttctaccctgtggtatccgtggttctaccctgtggtatccgtggttctaccctgtggta

SEQ ID NO: 112 - DNA, 63 bp, Artificial Sequence
  Description of Artificial Sequence Synthetic oligonucleotide
ccatctttaccagacagtgttccatctttaccagacagtgttccatctttaccagacagtgtt

SEQ ID NO: 113 - DNA, 60 bp, Artificial Sequence
  Description of Artificial Sequence Synthetic oligonucleotide
gtagtgctttctactttatggtagtgctttctactttatggtagtgctttctactttatg

SEQ ID NO: 114 - DNA, 66 bp, Artificial Sequence
  Description of Artificial Sequence Synthetic oligonucleotide
ccataaagtaggaaacactacaccataaagtaggaaacactacaccataaagtaggaaacactaca

SEQ ID NO: 115 - DNA, 72 bp, Artificial Sequence
  Description of Artificial Sequence Synthetic oligonucleotide
taagagctacagtgcttcatctcataagagctacagtgcttcatctcataagagctacagtgcttcatctca

SEQ ID NO: 116 - DNA, 66 bp, Artificial Sequence
  Description of Artificial Sequence Synthetic oligonucleotide
ctagtacatcatctatactgtactagtacatcatctatactgtactagtacatcatctatactgta

SEQ ID NO: 117 - DNA, 72 bp, Artificial Sequence
  Description of Artificial Sequence Synthetic oligonucleotide
aagggattcctgggaaaactggacaagggattcctgggaaaactggacaagggattcctgggaaaactggac

SEQ ID NO: 118 - DNA, 69 bp, Artificial Sequence
  Description of Artificial Sequence Synthetic oligonucleotide
aaacccatggaattcagttctcaaaacccatggaattcagttctcaaaacccatggaattcagttctca

SEQ ID NO: 119 - DNA, 69 bp, Artificial Sequence
  Description of Artificial Sequence Synthetic oligonucleotide
taacccatggaattcagttctcataacccatggaattcagttctcataacccatggaattcagttctca

SEQ ID NO: 120 - DNA, 63 bp, Artificial Sequence
  Description of Artificial Sequence Synthetic oligonucleotide
ggcagaagcatttccacacacggcagaagcatttccacacacggcagaagcatttccacacac

SEQ ID NO: 121 - DNA, 66 bp, Artificial Sequence
  Description of Artificial Sequence Synthetic oligonucleotide
acaaagttctgtagtgcactgaacaaagttctgtagtgcactgaacaaagttctgtagtgcactga

SEQ ID NO: 122 - DNA, 66 bp, Artificial Sequence
  Description of Artificial Sequence Synthetic oligonucleotide
acaaagttctgtgatgcactgaacaaagttctgtgatgcactgaacaaagttctgtgatgcactga

SEQ ID NO: 123 - DNA, 66 bp, Artificial Sequence
  Description of Artificial Sequence Synthetic oligonucleotide
ggagtgaagacacggagccagaggagtgaagacacggagccagaggagtgaagacacggagccaga

SEQ ID NO: 124 - DNA, 69 bp, Artificial Sequence
  Description of Artificial Sequence Synthetic oligonucleotide
acactggtacaagggttgggagaacactggtacaagggttgggagaacactggtacaagggttgggaga

SEQ ID NO: 125 - DNA, 69 bp, Artificial Sequence
  Description of Artificial Sequence Synthetic oligonucleotide
gcactggtacaagggttgggagagcactggtacaagggttgggagagcactggtacaagggttgggaga

SEQ ID NO: 126 - DNA, 66 bp, Artificial Sequence
  Description of Artificial Sequence Synthetic oligonucleotide
acctcaaggagcctcagtctagacctcaaggagcctcagtctagacctcaaggagcctcagtctag

SEQ ID NO: 127 - DNA, 63 bp, Artificial Sequence
  Description of Artificial Sequence Synthetic oligonucleotide
ccaagttctgtcatgcactgaccaagttctgtcatgcactgaccaagttctgtcatgcactga

SEQ ID NO: 128 - DNA, 60 bp, Artificial Sequence
  Description of Artificial Sequence Synthetic oligonucleotide
tcacttttgtgactatgcaatcacttttgtgactatgcaatcacttttgtgactatgcaa

SEQ ID NO: 129 - DNA, 66 bp, Artificial Sequence
  Description of Artificial Sequence Synthetic oligonucleotide
cgaaggcaacacggataacctacgaaggcaacacggataacctacgaaggcaacacggataaccta

SEQ ID NO: 130 - DNA, 66 bp, Artificial Sequence
  Description of Artificial Sequence Synthetic oligonucleotide
cccctatcacaattagcattaacccctatcacaattagcattaacccctatcacaattagcattaa

SEQ ID NO: 131 - DNA, 69 bp, Artificial Sequence
  Description of Artificial Sequence Synthetic oligonucleotide
aacaaccagctaagacactgccaaacaaccagctaagacactgccaaacaaccagctaagacactgcca

SEQ ID NO: 132 - DNA, 69 bp, Artificial Sequence
  Description of Artificial Sequence Synthetic oligonucleotide
actcaccgacagcgttgaatgttactcaccgacagcgttgaatgttactcaccgacagcgttgaatgtt

SEQ ID NO: 133 - DNA, 72 bp, Artificial Sequence
  Description of Artificial Sequence Synthetic oligonucleotide
aacccaccgacagcaatgaatgttaacccaccgacagcaatgaatgttaacccaccgacagcaatgaatgtt

SEQ ID NO: 134 - DNA, 66 bp, Artificial Sequence
  Description of Artificial Sequence Synthetic oligonucleotide
actcaccgacaggttgaatgttactcaccgacaggttgaatgttactcaccgacaggttgaatgtt

SEQ ID NO: 135 - DNA, 66 bp, Artificial Sequence
  Description of Artificial Sequence Synthetic oligonucleotide
tgtgagttctaccattgccaaatgtgagttctaccattgccaaatgtgagttctaccattgccaaa

SEQ ID NO: 136 - DNA, 69 bp, Artificial Sequence
  Description of Artificial Sequence Synthetic oligonucleotide
cagtgaattctaccagtgccatacagtgaattctaccagtgccatacagtgaattctaccagtgccata

SEQ ID NO: 137 - DNA, 66 bp, Artificial Sequence
  Description of Artificial Sequence Synthetic oligonucleotide
acccttatcagttctccgtccaacccttatcagttctccgtccaacccttatcagttctccgtcca

SEQ ID NO: 138 - DNA, 54 bp, Artificial Sequence
  Description of Artificial Sequence Synthetic oligonucleotide
gaactgcctttctctccagaactgcctttctctccagaactgcctttctctcca

SEQ ID NO: 139 - DNA, 69 bp, Artificial Sequence
  Description of Artificial Sequence Synthetic oligonucleotide
aagcccaaaaggagaattctttgaagcccaaaaggagaattctttgaagcccaaaaggagaattctttg

SEQ ID NO: 140 - DNA, 66 bp, Artificial Sequence
  Description of Artificial Sequence Synthetic oligonucleotide
ccggctgcaacacaagacacgaccggctgcaacacaagacacgaccggctgcaacacaagacacga

SEQ ID NO: 141 - DNA, 66 bp, Artificial Sequence
  Description of Artificial Sequence Synthetic oligonucleotide
accctccaccatgcaagggatgaccctccaccatgcaagggatgaccctccaccatgcaagggatg

SEQ ID NO: 142 - DNA, 69 bp, Artificial Sequence
  Description of Artificial Sequence Synthetic oligonucleotide
actgatatcagctcagtaggcacactgatatcagctcagtaggcacactgatatcagctcagtaggcac

SEQ ID NO: 143 - DNA, 66 bp, Artificial Sequence
  Description of Artificial Sequence Synthetic oligonucleotide
acctaatatatcaaacatatcaacctaatatatcaaacatatcaacctaatatatcaaacatatca

SEQ ID NO: 144 - DNA, 66 bp, Artificial Sequence
  Description of Artificial Sequence Synthetic oligonucleotide
agctgcttttgggattccgttgagctgcttttgggattccgttgagctgcttttgggattccgttg

SEQ ID NO: 145 - DNA, 63 bp, Artificial Sequence
  Description of Artificial Sequence Synthetic oligonucleotide
ggctgtcaattcataggtcagggctgtcaattcataggtcagggctgtcaattcataggtcag

SEQ ID NO: 146 - DNA, 63 bp, Artificial Sequence
  Description of Artificial Sequence Synthetic oligonucleotide
ctgggactttgtaggccagttctgggactttgtaggccagttctgggactttgtaggccagtt

SEQ ID NO: 147 - DNA, 66 bp, Artificial Sequence
  Description of Artificial Sequence Synthetic oligonucleotide
tccacatggagttgctgttacatccacatggagttgctgttacatccacatggagttgctgttaca

SEQ ID NO: 148 - DNA, 63 bp, Artificial Sequence
  Description of Artificial Sequence Synthetic oligonucleotide
gccaatatttctgtgctgctagccaatatttctgtgctgctagccaatatttctgtgctgcta

SEQ ID NO: 149 - DNA, 66 bp, Artificial Sequence
  Description of Artificial Sequence Synthetic oligonucleotide
cccaacaacatgaaactacctacccaacaacatgaaactacctacccaacaacatgaaactaccta

SEQ ID NO: 150 - DNA, 66 bp, Artificial Sequence
  Description of Artificial Sequence Synthetic oligonucleotide
gctgggtggagaaggtggtgaagctgggtggagaaggtggtgaagctgggtggagaaggtggtgaa

SEQ ID NO: 151 - DNA, 57 bp, Artificial Sequence
  Description of Artificial Sequence Synthetic oligonucleotide
cctatctcccctctggacccctatctcccctctggacccctatctcccctctggacc

SEQ ID NO: 152 - DNA, 69 bp, Artificial Sequence
  Description of Artificial Sequence Synthetic oligonucleotide
gaacaggtagtctgaacactggggaacaggtagtctgaacactggggaacaggtagtctgaacactggg

SEQ ID NO: 153 - DNA, 66 bp, Artificial Sequence
  Description of Artificial Sequence Synthetic oligonucleotide
aaccaatgtgcagactactgtaaaccaatgtgcagactactgtaaaccaatgtgcagactactgta

SEQ ID NO: 154 - DNA, 69 bp, Artificial Sequence
  Description of Artificial Sequence Synthetic oligonucleotide
gaacagatagtctaaacactggggaacagatagtctaaacactggggaacagatagtctaaacactggg

SEQ ID NO: 155 - DNA, 69 bp, Artificial Sequence
  Description of Artificial Sequence Synthetic oligonucleotide
gtcatcattaccaggcagtattagtcatcattaccaggcagtattagtcatcattaccaggcagtatta

SEQ ID NO: 156 - DNA, 63 bp, Artificial Sequence
  Description of Artificial Sequence Synthetic oligonucleotide
agaacaatgccttactgagtaagaacaatgccttactgagtaagaacaatgccttactgagta

SEQ ID NO: 157 - DNA, 66 bp, Artificial Sequence
  Description of Artificial Sequence Synthetic oligonucleotide
tcttcccatgcgctatacctcttcttcccatgcgctatacctcttcttcccatgcgctatacctct

SEQ ID NO: 158 - DNA, 66 bp, Artificial Sequence
  Description of Artificial Sequence Synthetic oligonucleotide
tctagtggtcctaaacatttcatctagtggtcctaaacatttcatctagtggtcctaaacatttca

SEQ ID NO: 159 - DNA, 69 bp, Artificial Sequence
  Description of Artificial Sequence Synthetic oligonucleotide
caggcataggatgacaaagggaacaggcataggatgacaaagggaacaggcataggatgacaaagggaa

SEQ ID NO: 160 - DNA, 66 bp, Artificial Sequence
  Description of Artificial Sequence Synthetic oligonucleotide
cagactccggtggaatgaaggacagactccggtggaatgaaggacagactccggtggaatgaagga

SEQ ID NO: 161 - DNA, 66 bp, Artificial Sequence
  Description of Artificial Sequence Synthetic oligonucleotide
ccacacacttccttacattccaccacacacttccttacattccaccacacacttccttacattcca

SEQ ID NO: 162 - DNA, 69 bp, Artificial Sequence
  Description of Artificial Sequence Synthetic oligonucleotide
gagggaggagagccaggagaagcgagggaggagagccaggagaagcgagggaggagagccaggagaagc

SEQ ID NO: 163 - DNA, 66 bp, Artificial Sequence
  Description of Artificial Sequence Synthetic oligonucleotide
acaagctttttgctcgtcttatacaagctttttgctcgtcttatacaagctttttgctcgtcttat

SEQ ID NO: 164 - DNA, 63 bp, Artificial Sequence
  Description of Artificial Sequence Synthetic oligonucleotide
cagccgctgtcacacgcacagcagccgctgtcacacgcacagcagccgctgtcacacgcacag

SEQ ID NO: 165 - DNA, 66 bp, Artificial Sequence
  Description of Artificial Sequence Synthetic oligonucleotide
aggcgaaggatgacaaagggaaaggcgaaggatgacaaagggaaaggcgaaggatgacaaagggaa

SEQ ID NO: 166 - DNA, 63 bp, Artificial Sequence
  Description of Artificial Sequence Synthetic oligonucleotide
ggccgtgactggagactgttaggccgtgactggagactgttaggccgtgactggagactgtta

SEQ ID NO: 167 - DNA, 66 bp, Artificial Sequence
  Description of Artificial Sequence Synthetic oligonucleotide
ggtacaatcaacggtcgatggtggtacaatcaacggtcgatggtggtacaatcaacggtcgatggt

SEQ ID NO: 168 - DNA, 63 bp, Artificial Sequence
  Description of Artificial Sequence Synthetic oligonucleotide
ctgcctgtctgtgcctgctgtctgcctgtctgtgcctgctgtctgcctgtctgtgcctgctgt

SEQ ID NO: 169 - DNA, 63 bp, Artificial Sequence
  Description of Artificial Sequence Synthetic oligonucleotide
gtctgtcaattcataggtcatgtctgtcaattcataggtcatgtctgtcaattcataggtcat

SEQ ID NO: 170 - DNA, 63 bp, Artificial Sequence
  Description of Artificial Sequence Synthetic oligonucleotide
cacagttgccagctgagattacacagttgccagctgagattacacagttgccagctgagatta

SEQ ID NO: 171 - DNA, 72 bp, Artificial Sequence
  Description of Artificial Sequence Synthetic oligonucleotide
atccaatcagttcctgatgcagtaatccaatcagttcctgatgcagtaatccaatcagttcctgatgcagta

SEQ ID NO: 172 - DNA, 72 bp, Artificial Sequence
  Description of Artificial Sequence Synthetic oligonucleotide
atccagtcagttcctgatgcagtaatccagtcagttcctgatgcagtaatccagtcagttcctgatgcagta

SEQ ID NO: 173 - DNA, 66 bp, Artificial Sequence
  Description of Artificial Sequence Synthetic oligonucleotide
cacatggttagatcaagcacaacacatggttagatcaagcacaacacatggttagatcaagcacaa

SEQ ID NO: 174 - DNA, 63 bp, Artificial Sequence
  Description of Artificial Sequence Synthetic oligonucleotide
agaattgcgtttggacaatcaagaattgcgtttggacaatcaagaattgcgtttggacaatca

SEQ ID NO: 175 - DNA, 63 bp, Artificial Sequence
  Description of Artificial Sequence Synthetic oligonucleotide
aaagtgtcagatacggtgtggaaagtgtcagatacggtgtggaaagtgtcagatacggtgtgg

SEQ ID NO: 176 - DNA, 69 bp, Artificial Sequence
  Description of Artificial Sequence Synthetic oligonucleotide
gaaacccagcagacaatgtagctgaaacccagcagacaatgtagctgaaacccagcagacaatgtagct

SEQ ID NO: 177 - DNA, 72 bp, Artificial Sequence
  Description of Artificial Sequence Synthetic oligonucleotide
gagacccagtagccagatgtagctgagacccagtagccagatgtagctgagacccagtagccagatgtagct

SEQ ID NO: 178 - DNA, 63 bp, Artificial Sequence
  Description of Artificial Sequence Synthetic oligonucleotide
ggggtatttgacaaactgacaggggtatttgacaaactgacaggggtatttgacaaactgaca

SEQ ID NO: 179 - DNA, 69 bp, Artificial Sequence
  Description of Artificial Sequence Synthetic oligonucleotide
taaacggaaccactagtgacttgtaaacggaaccactagtgacttgtaaacggaaccactagtgacttg

SEQ ID NO: 180 - DNA, 60 bp, Artificial Sequence
  Description of Artificial Sequence Synthetic oligonucleotide
ccagcagcacctggggcagtccagcagcacctggggcagtccagcagcacctggggcagt

SEQ ID NO: 181 - DNA, 69 bp, Artificial Sequence
  Description of Artificial Sequence Synthetic oligonucleotide
acaccaatgccctaggggatgcgacaccaatgccctaggggatgcgacaccaatgccctaggggatgcg

SEQ ID NO: 182 - DNA, 63 bp, Artificial Sequence
  Description of Artificial Sequence Synthetic oligonucleotide
actggaggaagggcccagaggactggaggaagggcccagaggactggaggaagggcccagagg

SEQ ID NO: 183 - DNA, 69 bp, Artificial Sequence
  Description of Artificial Sequence Synthetic oligonucleotide
aagaaaggcagcaggtcgtatagaagaaaggcagcaggtcgtatagaagaaaggcagcaggtcgtatag

SEQ ID NO: 184 - DNA, 66 bp, Artificial Sequence
  Description of Artificial Sequence Synthetic oligonucleotide
acggaagggcagagagggccagacggaagggcagagagggccagacggaagggcagagagggccag

SEQ ID NO: 185 - DNA, 66 bp, Artificial Sequence
  Description of Artificial Sequence Synthetic oligonucleotide
aaaaaggttagctgggtgtgttaaaaaggttagctgggtgtgttaaaaaggttagctgggtgtgtt

SEQ ID NO: 186 - DNA, 72 bp, Artificial Sequence
  Description of Artificial Sequence Synthetic oligonucleotide
tgtccgtggttctaccctgtggtatgtccgtggttctaccctgtggtatgtccgtggttctaccctgtggta

SEQ ID NO: 187 - DNA, 69 bp, Artificial Sequence
  Description of Artificial Sequence Synthetic oligonucleotide
acatttttcgttattgctcttgaacatttttcgttattgctcttgaacatttttcgttattgctcttga

SEQ ID NO: 188 - DNA, 69 bp, Artificial Sequence
  Description of Artificial Sequence Synthetic oligonucleotide
tcaacaaaatcactgatgctggatcaacaaaatcactgatgctggatcaacaaaatcactgatgctgga

SEQ ID NO: 189 - DNA, 72 bp, Artificial Sequence
  Description of Artificial Sequence Synthetic oligonucleotide
gacgggtgcgatttctgtgtgagagacgggtgcgatttctgtgtgagagacgggtgcgatttctgtgtgaga

SEQ ID NO: 190 - DNA, 69 bp, Artificial Sequence
  Description of Artificial Sequence Synthetic oligonucleotide
acagtcaggctttggctagatcaacagtcaggctttggctagatcaacagtcaggctttggctagatca

SEQ ID NO: 191 - DNA, 63 bp, Artificial Sequence
  Description of Artificial Sequence Synthetic oligonucleotide
gcactggactaggggtcagcagcactggactaggggtcagcagcactggactaggggtcagca

SEQ ID NO: 192 - DNA, 66 bp, Artificial Sequence
  Description of Artificial Sequence Synthetic oligonucleotide
atgctttttggggtaagggcttatgctttttggggtaagggcttatgctttttggggtaagggctt

SEQ ID NO: 193 - DNA, 69 bp, Artificial Sequence
  Description of Artificial Sequence Synthetic oligonucleotide
aaaaagtgcccccatagtttgagaaaaagtgcccccatagtttgagaaaaagtgcccccatagtttgag

SEQ ID NO: 194 - DNA, 66 bp, Artificial Sequence
  Description of Artificial Sequence Synthetic oligonucleotide
agagagggcctccactttgatgagagagggcctccactttgatgagagagggcctccactttgatg

SEQ ID NO: 195 - DNA, 69 bp, Artificial Sequence
  Description of Artificial Sequence Synthetic oligonucleotide
ggcacacaaagtggaagcactttggcacacaaagtggaagcactttggcacacaaagtggaagcacttt

SEQ ID NO: 196 - DNA, 66 bp, Artificial Sequence
  Description of Artificial Sequence Synthetic oligonucleotide
caaaagagcccccagtttgagtcaaaagagcccccagtttgagtcaaaagagcccccagtttgagt

SEQ ID NO: 197 - DNA, 69 bp, Artificial Sequence
  Description of Artificial Sequence Synthetic oligonucleotide
acactcaaaacctggcggcacttacactcaaaacctggcggcacttacactcaaaacctggcggcactt

SEQ ID NO: 198 - DNA, 66 bp, Artificial Sequence
  Description of Artificial Sequence Synthetic oligonucleotide
acactacaaactctgcggcactacactacaaactctgcggcactacactacaaactctgcggcact

SEQ ID NO: 199 - DNA, 66 bp, Artificial Sequence
  Description of Artificial Sequence Synthetic oligonucleotide
acacacaaaagggaagcactttacacacaaaagggaagcactttacacacaaaagggaagcacttt

SEQ ID NO: 200 - DNA, 69 bp, Artificial Sequence
  Description of Artificial Sequence Synthetic oligonucleotide
agactcaaaagtagtagcactttagactcaaaagtagtagcactttagactcaaaagtagtagcacttt

SEQ ID NO: 201 - DNA, 63 bp, Artificial Sequence
  Description of Artificial Sequence Synthetic oligonucleotide
acaggattgagggggggccctacaggattgagggggggccctacaggattgagggggggccct

SEQ ID NO: 202 - DNA, 63 bp, Artificial Sequence
  Description of Artificial Sequence Synthetic oligonucleotide
catgcacatgcacacatacatcatgcacatgcacacatacatcatgcacatgcacacatacat

SEQ ID NO: 203 - DNA, 66 bp, Artificial Sequence
  Description of Artificial Sequence Synthetic oligonucleotide
ggaagaacagccctcctctgccggaagaacagccctcctctgccggaagaacagccctcctctgcc

SEQ ID NO: 204 - DNA, 66 bp, Artificial Sequence
  Description of Artificial Sequence Synthetic oligonucleotide
atgtatgtgggacggtaaaccaatgtatgtgggacggtaaaccaatgtatgtgggacggtaaacca

SEQ ID NO: 205 - DNA, 66 bp, Artificial Sequence
  Description of Artificial Sequence Synthetic oligonucleotide
gaagagagcttgcccttgcatagaagagagcttgcccttgcatagaagagagcttgcccttgcata

SEQ ID NO: 206 - DNA, 69 bp, Artificial Sequence
  Description of Artificial Sequence Synthetic oligonucleotide
gctttgacaatactattgcactggctttgacaatactattgcactggctttgacaatactattgcactg

SEQ ID NO: 207 - DNA, 69 bp, Artificial Sequence
  Description of Artificial Sequence Synthetic oligonucleotide
tcaccaaaacatggaagcacttatcaccaaaacatggaagcacttatcaccaaaacatggaagcactta

SEQ ID NO: 208 - DNA, 69 bp, Artificial Sequence
  Description of Artificial Sequence Synthetic oligonucleotide
ttcgccctctcaacccagcttttttcgccctctcaacccagcttttttcgccctctcaacccagctttt

SEQ ID NO: 209 - DNA, 63 bp, Artificial Sequence
  Description of Artificial Sequence Synthetic oligonucleotide
gaacccacaatccctggcttagaacccacaatccctggcttagaacccacaatccctggctta

SEQ ID NO: 210 - DNA, 60 bp, Artificial Sequence
  Description of Artificial Sequence Synthetic oligonucleotide
tgttgcagcgcttcatgttttgttgcagcgcttcatgttttgttgcagcgcttcatgttt

SEQ ID NO: 211 - DNA, 66 bp, Artificial Sequence
  Description of Artificial Sequence Synthetic oligonucleotide
agaggtcgaccgtgtaatgtgcagaggtcgaccgtgtaatgtgcagaggtcgaccgtgtaatgtgc

SEQ ID NO: 212 - DNA, 66 bp, Artificial Sequence
  Description of Artificial Sequence Synthetic oligonucleotide
ccagcagcacctggggcagtggccagcagcacctggggcagtggccagcagcacctggggcagtgg

SEQ ID NO: 213 - DNA, 69 bp, Artificial Sequence; identical to SEQ ID NO: 181
  Description of Artificial Sequence Synthetic oligonucleotide

SEQ ID NO: 214 - DNA, 69 bp, Artificial Sequence
  Description of Artificial Sequence Synthetic oligonucleotide
acacttactgagcacctactaggacacttactgagcacctactaggacacttactgagcacctactagg

SEQ ID NO: 215 - DNA, 63 bp, Artificial Sequence; identical to SEQ ID NO: 182
  Description of Artificial Sequence Synthetic oligonucleotide

SEQ ID NO: 216 - DNA, 57 bp, Artificial Sequence
  Description of Artificial Sequence Synthetic oligonucleotide
accctcatgcccctcaaggaccctcatgcccctcaaggaccctcatgcccctcaagg

SEQ ID NO: 217 - DNA, 66 bp, Artificial Sequence; identical to SEQ ID NO: 184
  Description of Artificial Sequence Synthetic oligonucleotide

SEQ ID NO: 218 - DNA, 66 bp, Artificial Sequence; identical to SEQ ID NO: 185
  Description of Artificial Sequence Synthetic oligonucleotide

SEQ ID NO: 219 - DNA, 72 bp, Artificial Sequence
  Description of Artificial Sequence Synthetic oligonucleotide
ttctctgcaggccctgtgctttgcttctctgcaggccctgtgctttgcttctctgcaggccctgtgctttgc

SEQ ID NO: 220 - DNA, 63 bp, Artificial Sequence
  Description of Artificial Sequence Synthetic oligonucleotide
ttctaggataggcccaggggcttctaggataggcccaggggcttctaggataggcccaggggc

SEQ ID NO: 221 - DNA, 57 bp, Artificial Sequence
  Description of Artificial Sequence Synthetic oligonucleotide
tggcgactctggtgggacctggcgactctggtgggacctggcgactctggtgggacc

SEQ ID NO: 222 - DNA, 60 bp, Artificial Sequence
  Description of Artificial Sequence Synthetic oligonucleotide
aaaagtaactagcacaccacaaaagtaactagcacaccacaaaagtaactagcacaccac

SEQ ID NO: 223 - DNA, 57 bp, Artificial Sequence
  Description of Artificial Sequence Synthetic oligonucleotide
cacatctctgcaccgtttacacatctctgcaccgtttacacatctctgcaccgttta

SEQ ID NO: 224 - DNA, 69 bp, Artificial Sequence; identical to SEQ ID NO: 187
  Description of Artificial Sequence Synthetic oligonucleotide

SEQ ID NO: 225 - DNA, 63 bp, Artificial Sequence
  Description of Artificial Sequence Synthetic oligonucleotide
agactagatatggaagggtgaagactagatatggaagggtgaagactagatatggaagggtga

SEQ ID NO: 226 - DNA, 69 bp, Artificial Sequence
  Description of Artificial Sequence Synthetic oligonucleotide
aaaggcatcatataggagctgaaaaaggcatcatataggagctgaaaaaggcatcatataggagctgaa

SEQ ID NO: 227 - DNA, 69 bp, Artificial Sequence; identical to SEQ ID NO: 188
  Description of Artificial Sequence Synthetic oligonucleotide

SEQ ID NO: 228 - DNA, 69 bp, Artificial Sequence
  Description of Artificial Sequence Synthetic oligonucleotide
aatgagctcctggaggacagggaaatgagctcctggaggacagggaaatgagctcctggaggacaggga

SEQ ID NO: 229 - DNA, 69 bp, Artificial Sequence
  Description of Artificial Sequence Synthetic oligonucleotide
ggctataaagtaactgagacggaggctataaagtaactgagacggaggctataaagtaactgagacgga

SEQ ID NO: 230 - DNA, 63 bp, Artificial Sequence
  Description of Artificial Sequence Synthetic oligonucleotide
actgaccgaccgaccgatcgaactgaccgaccgaccgatcgaactgaccgaccgaccgatcga

SEQ ID NO: 231 - DNA, 72 bp, Artificial Sequence; identical to SEQ ID NO: 189
  Description of Artificial Sequence Synthetic oligonucleotide

SEQ ID NO: 232 - DNA, 63 bp, Artificial Sequence
  Description of Artificial Sequence Synthetic oligonucleotide
aactgggcacacggagggagaaactgggcacacggagggagaaactgggcacacggagggaga

SEQ ID NO: 233 - DNA, 69 bp, Artificial Sequence
  Description of Artificial Sequence Synthetic oligonucleotide
acggtcaggctttggctagatcaacggtcaggctttggctagatcaacggtcaggctttggctagatca

SEQ ID NO: 234 - DNA, 63 bp, Artificial Sequence; identical to SEQ ID NO: 191
  Description of Artificial Sequence Synthetic oligonucleotide

SEQ ID NO: 235 - DNA, 75 bp, Artificial Sequence
  Description of Artificial Sequence Synthetic oligonucleotide
ttagaggcaggcactcaggcagacattagaggcaggcactcaggcagacattagaggcaggcactcaggcagaca

SEQ ID NO: 236 - DNA, 54 bp, Artificial Sequence
  Description of Artificial Sequence Synthetic oligonucleotide
tggcgacccagagggacatggcgacccagagggacatggcgacccagagggaca

SEQ ID NO: 237 - DNA, 75 bp, Artificial Sequence
  Description of Artificial Sequence Synthetic oligonucleotide
actggagtggggtaaagggtgggcaactggagtggggtaaagggtgggcaactggagtggggtaaagggtgggca

SEQ ID NO: 238 - DNA, 66 bp, Artificial Sequence
  Description of Artificial Sequence Synthetic oligonucleotide
agaggttaagacagcagggctgagaggttaagacagcagggctgagaggttaagacagcagggctg

SEQ ID NO: 239 - DNA, 75 bp, Artificial Sequence
  Description of Artificial Sequence Synthetic oligonucleotide
gtgaaagtgtatgggctttgtgaatgtgaaagtgtatgggctttgtgaatgtgaaagtgtatgggctttgtgaat

SEQ ID NO: 240 - DNA, 75 bp, Artificial Sequence
  Description of Artificial Sequence Synthetic oligonucleotide
acaggctcaaagggctcctcagggaacaggctcaaagggctcctcagggaacaggctcaaagggctcctcaggga

SEQ ID NO: 241 - DNA, 63 bp, Artificial Sequence
  Description of Artificial Sequence Synthetic oligonucleotide
tactatgcaacctactactcttactatgcaacctactactcttactatgcaacctactactct

SEQ ID NO: 242 - DNA, 75 bp, Artificial Sequence
  Description of Artificial Sequence Synthetic oligonucleotide
gaccagtgctctaacccctgagctagaccagtgctctaacccctgagctagaccagtgctctaacccctgagcta

SEQ ID NO: 243 - DNA, 60 bp, Artificial Sequence
  Description of Artificial Sequence Synthetic oligonucleotide
tcacttataacagtaatgtatcacttataacagtaatgtatcacttataacagtaatgta

SEQ ID NO: 244 - DNA, 69 bp, Artificial Sequence
  Description of Artificial Sequence Synthetic oligonucleotide
tcatacccctgtaaaatgctccttcatacccctgtaaaatgctccttcatacccctgtaaaatgctcct

SEQ ID NO: 245 - DNA, 48 bp, Artificial Sequence
  Description of Artificial Sequence Synthetic oligonucleotide
aaatgttactgctcaaaaatgttactgctcaaaaatgttactgctcaa

SEQ ID NO: 246 - DNA, 54 bp, Artificial Sequence
  Description of Artificial Sequence Synthetic oligonucleotide
atcatacaatcaacgcatatcatacaatcaacgcatatcatacaatcaacgcat

SEQ ID NO: 247 - DNA, 66 bp, Artificial Sequence
  Description of Artificial Sequence Synthetic oligonucleotide
aaaacagacccgttcaatgtgaaaaacagacccgttcaatgtgaaaaacagacccgttcaatgtga

SEQ ID NO: 248 - DNA, 63 bp, Artificial Sequence
  Description of Artificial Sequence Synthetic oligonucleotide
gcaaccccagaaccaaaaaaggcaaccccagaaccaaaaaaggcaaccccagaaccaaaaaag

SEQ ID NO: 249 - DNA, 54 bp, Artificial Sequence
  Description of Artificial Sequence Synthetic oligonucleotide
agaaacgtccaatgtagaagaaacgtccaatgtagaagaaacgtccaatgtaga

SEQ ID NO: 250 - DNA, 63 bp, Artificial Sequence
  Description of Artificial Sequence Synthetic oligonucleotide
ctacaatagcgtaaaaacactctacaatagcgtaaaaacactctacaatagcgtaaaaacact

SEQ ID NO: 251 - DNA, 66 bp, Artificial Sequence
  Description of Artificial Sequence Synthetic oligonucleotide
ggactgaagaaacggagacagaggactgaagaaacggagacagaggactgaagaaacggagacaga

SEQ ID NO: 252 - DNA, 69 bp, Artificial Sequence
  Description of Artificial Sequence Synthetic oligonucleotide
acaatggtacaacggttggcagaacaatggtacaacggttggcagaacaatggtacaacggttggcaga

SEQ ID NO: 253 - DNA, 66 bp, Artificial Sequence
  Description of Artificial Sequence Synthetic oligonucleotide
accattatcacttctccgtacaaccattatcacttctccgtacaaccattatcacttctccgtaca

SEQ ID NO: 254 - DNA, 54 bp, Artificial Sequence
  Description of Artificial Sequence Synthetic oligonucleotide
gaactccctttatctacagaactccctttatctacagaactccctttatctaca

SEQ ID NO: 255 - DNA, 57 bp, Artificial Sequence
  Description of Artificial Sequence Synthetic oligonucleotide
cctatatcccatctgcacccctatatcccatctgcacccctatatcccatctgcacc

SEQ ID NO: 256 - DNA, 69 bp, Artificial Sequence
  Description of Artificial Sequence Synthetic oligonucleotide
gaaaaggtactctgaacaatggggaaaaggtactctgaacaatggggaaaaggtactctgaacaatggg

SEQ ID NO: 257 - DNA, 66 bp, Artificial Sequence
  Description of Artificial Sequence Synthetic oligonucleotide
aggagaagcatgacaaacggaaaggagaagcatgacaaacggaaaggagaagcatgacaaacggaa

SEQ ID NO: 258 - DNA, 63 bp, Artificial Sequence
  Description of Artificial Sequence Synthetic oligonucleotide
ggcagtgactcgagaatgttaggcagtgactcgagaatgttaggcagtgactcgagaatgtta

SEQ ID NO: 259 - DNA, 69 bp, Artificial Sequence
  Description of Artificial Sequence Synthetic oligonucleotide
taaaaggaacaactagtcacttgtaaaaggaacaactagtcacttgtaaaaggaacaactagtcacttg

SEQ ID NO: 260 - DNA, 66 bp, Artificial Sequence
  Description of Artificial Sequence Synthetic oligonucleotide
atgatttttggcgtaagcgcttatgatttttggcgtaagcgcttatgatttttggcgtaagcgctt

SEQ ID NO: 261 - DNA, 69 bp, Artificial Sequence
  Description of Artificial Sequence Synthetic oligonucleotide
aaaaactgccaccatactttgagaaaaactgccaccatactttgagaaaaactgccaccatactttgag

SEQ ID NO: 262 - DNA, 69 bp, Artificial Sequence
  Description of Artificial Sequence Synthetic oligonucleotide
gctttcacaataatattgaactggctttcacaataatattgaactggctttcacaataatattgaactg

SEQ ID NO: 263 - DNA, 69 bp, Artificial Sequence
  Description of Artificial Sequence Synthetic oligonucleotide
tcacaaaaacatcgaagcaattatcacaaaaacatcgaagcaattatcacaaaaacatcgaagcaatta

SEQ ID NO: 264 - DNA, 63 bp, Artificial Sequence
  Description of Artificial Sequence Synthetic oligonucleotide
ttataggatacgcccagcggcttataggatacgcccagcggcttataggatacgcccagcggc

SEQ ID NO: 265 - DNA, 57 bp, Artificial Sequence
  Description of Artificial Sequence Synthetic oligonucleotide
tggagactatggtggcacctggagactatggtggcacctggagactatggtggcacc

SEQ ID NO: 266 - DNA, 63 bp, Artificial Sequence
  Description of Artificial Sequence Synthetic oligonucleotide
gcaatggaatagggctcagcagcaatggaatagggctcagcagcaatggaatagggctcagca

SEQ ID NO: 267 - DNA, 75 bp, Artificial Sequence
  Description of Artificial Sequence Synthetic oligonucleotide
ttagacgcaggaactcaggaagacattagacgcaggaactcaggaagacattagacgcaggaactcaggaagaca

SEQ ID NO: 268 - DNA, 75 bp, Artificial Sequence
  Description of Artificial Sequence Synthetic oligonucleotide
gacaagtgctctaaaccctcagctagacaagtgctctaaaccctcagctagacaagtgctctaaaccctcagcta

SEQ ID NO: 269 - DNA, 24 bp, Artificial Sequence
  Description of Artificial Sequence Synthetic oligonucleotide
actacctgcactgtaagcactttg

SEQ ID NO: 270 - DNA, 22 bp, Artificial Sequence
  Description of Artificial Sequence Synthetic oligonucleotide
tatctgcactagatgcacctta

SEQ ID NO: 271 - DNA, 23 bp, Artificial Sequence
  Description of Artificial Sequence Synthetic oligonucleotide
tcagttttgcatagatttgcaca

SEQ ID NO: 272 - DNA, 23 bp, Artificial Sequence
  Description of Artificial Sequence Synthetic oligonucleotide
ctacctgcactataagcacttta

SEQ ID NO: 273 - DNA, 22 bp, Artificial Sequence
  Description of Artificial Sequence Synthetic oligonucleotide
acaggccgggacaagtgcaata

SEQ ID NO: 274 - DNA, 23 bp, Artificial Sequence
  Description of Artificial Sequence Synthetic oligonucleotide
gctgagagtgtaggatgtttaca

SEQ ID NO: 275 - DNA, 22 bp, Artificial Sequence
  Description of Artificial Sequence Synthetic oligonucleotide
cgccaatatttacgtgctgcta

SEQ ID NO: 276 - DNA, 22 bp, Artificial Sequence
  Description of Artificial Sequence Synthetic oligonucleotide
gacatctgtcaccccattgatc

SEQ ID NO: 277 - DNA, 22 bp, Artificial Sequence
  Description of Artificial Sequence Synthetic oligonucleotide
tctgcagtcttggaagcttgac

SEQ ID NO: 278 - DNA, 22 bp, Artificial Sequence
  Description of Artificial Sequence Synthetic oligonucleotide
tgtgtgcatgagtccatgtgtg

SEQ ID NO: 279 - DNA, 23 bp, Artificial Sequence
  Description of Artificial Sequence Synthetic oligonucleotide
gcaaatcaaagtgcagattggag

SEQ ID NO: 280 - DNA, 25 bp, Artificial Sequence
  Description of Artificial Sequence Synthetic oligonucleotide
aaacgttctgaatgttctggattgt

SEQ ID NO: 281 - DNA, 19 bp, Artificial Sequence
  Description of Artificial Sequence Synthetic oligonucleotide
acctcggaaacccaccaag

SEQ ID NO: 282 - DNA, 19 bp, Artificial Sequence
  Description of Artificial Sequence Synthetic oligonucleotide
aaaggcaggctcgtcgttg

SEQ ID NO: 283 - DNA, 24 bp, Artificial Sequence
  Description of Artificial Sequence Synthetic oligonucleotide
ctcgactcttactctcacaaatgg

SEQ ID NO: 284 - DNA, 25 bp, Artificial Sequence
  Description of Artificial Sequence Synthetic oligonucleotide
tttaaacaggatatttacgttctgc

SEQ ID NO: 285 - DNA, 23 bp, Artificial Sequence
  Description of Artificial Sequence Synthetic oligonucleotide
ccaagctgaagtacaggcaaact

SEQ ID NO: 286 - DNA, 30 bp, Artificial Sequence
  Description of Artificial Sequence Synthetic oligonucleotide
ttgtcactacagatggtctaaaggttactt

SEQ ID NO: 287 - DNA, 19 bp, Artificial Sequence
  Description of Artificial Sequence Synthetic oligonucleotide
gctacatccgggactcacc

SEQ ID NO: 288 - DNA, 24 bp, Artificial Sequence
  Description of Artificial Sequence Synthetic oligonucleotide
cacagccttctcaagtcagctaaa

SEQ ID NO: 289 - DNA, 18 bp, Artificial Sequence
  Description of Artificial Sequence Synthetic oligonucleotide
tctccctgggactcgacg

SEQ ID NO: 290 - DNA, 24 bp, Artificial Sequence
  Description of Artificial Sequence Synthetic oligonucleotide
cgggataaagagttgtttctccaa

SEQ ID NO: 291 - DNA, 21 bp, Artificial Sequence
  Description of Artificial Sequence Synthetic oligonucleotide
gctactggtgcagttaggtcc

SEQ ID NO: 292 - DNA, 21 bp, Artificial Sequence
  Description of Artificial Sequence Synthetic oligonucleotide
gaggaaatcttcacatccacg

SEQ ID NO: 293 - DNA, 25 bp, Artificial Sequence
  Description of Artificial Sequence Synthetic oligonucleotide
tgggtggtctaacctagtgttatgg

SEQ ID NO: 294 - DNA, 20 bp, Artificial Sequence
  Description of Artificial Sequence Synthetic oligonucleotide
tccttgtctccacttcccca

SEQ ID NO: 295 - DNA, 20 bp, Artificial Sequence
  Description of Artificial Sequence Synthetic oligonucleotide
gctgccatcacagtacatgc

SEQ ID NO: 296 - RNA, 23 nt, Artificial Sequence
  Description of Artificial Sequence Synthetic oligonucleotide
aaaaccuuuugaccgagcguguu

SEQ ID NO: 297 - DNA, 24 bp, Artificial Sequence
  Description of Artificial Sequence Synthetic oligonucleotide
ctaaatggacctcatatctttgag

SEQ ID NO: 298 - DNA, 26 bp, Artificial Sequence
  Description of Artificial Sequence Synthetic oligonucleotide
gaaaacaagacaagatgtatttacac

SEQ ID NO: 299 - DNA, 35 bp, Artificial Sequence
  Description of Artificial Sequence Synthetic oligonucleotide
ggctagtctagaggctaaagagctttgtgatatac

SEQ ID NO: 300 - DNA, 38 bp, Artificial Sequence
  Description of Artificial Sequence Synthetic oligonucleotide
ggctagtctagaaaaaaatgtgcaaaactgcaaaattc

SEQ ID NO: 301 - DNA, 72 bp, Artificial Sequence
  Description of Artificial Sequence Synthetic oligonucleotide
ggctagtctagaggctaaagagctttgtgatatactggttcacatcctacccctgttctcttgtggcaacag

SEQ ID NO: 302 - DNA, 54 bp, Artificial Sequence
  Description of Artificial Sequence Synthetic oligonucleotide
ggctagtctagaaaaaaatgagaacaactgcaaaattcattgtaatagaatgtg

SEQ ID NO: 303 - DNA, 34 bp, Artificial Sequence
  Description of Artificial Sequence Synthetic oligonucleotide
ggctagtctagatgtgtgcatgagtccatgtgtg

SEQ ID NO: 304 - DNA, 35 bp, Artificial Sequence
  Description of Artificial Sequence Synthetic oligonucleotide
ggctagtctagagcaaatcaaagtgcagattggag

SEQ ID NO: 305 - DNA, 75 bp, Artificial Sequence
  Description of Artificial Sequence Synthetic oligonucleotide
ggctagtctagatgtgtgcatgagtccatgtgtgcgcgtgggggggctctaactggagtgtcggcccttttgctc

SEQ ID NO: 306 - DNA, 45 bp, Artificial Sequence
  Description of Artificial Sequence Synthetic oligonucleotide
ggctagtctagagcaaatcacactccagattggagggtggggcag

SEQ ID NO: 307 - DNA, 649 bp, Homo sapiens
tgtgtgcatgagtccatgtgtgcgcgtgggggggctctaactgcactttcggcccttttgctctgggggtcccacaaggcccagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggccccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctgcactttgatttgc

SEQ ID NO: 308 - DNA, 649 bp, Homo sapiens
tgtgtgcatgagtccatgtgtgcgcgtgggggggctctaactggagtgtcggcccttttgctctgggggtcccacaaggcccagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggccccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctggagtgtgatttgc

SEQ ID NO: 309 - RNA, 23 nt, Artificial Sequence
  Description of Artificial Sequence Synthetic oligonucleotide
caaaugcuuacagugcagguagu

SEQ ID NO: 310 - RNA, 22 nt, Artificial Sequence
  Description of Artificial Sequence Synthetic oligonucleotide
uaaaugcuuauagugcagguag

SEQ ID NO: 311 - RNA, 31 nt, Homo sapiens
ugggggggcucuaacugcacuuucggcccuu

SEQ ID NO: 312 - RNA, 31 nt, Pan troglodytes
ucggggggcucuaacugcacuuucggcccuu

SEQ ID NO: 313 - RNA, 39 nt, Mus sp.
uuggggugggggugggcucuaacugcacuuuuggugucc

SEQ ID NO: 314 - RNA, 38 nt, Rattus sp.
uugggguggggugggcucuaacagcacuuuuggugucc

SEQ ID NO: 315 - RNA, 32 nt, Canis familiaris
uggugggggcucuaacugcacuuuuggucucc

SEQ ID NO: 316 - RNA, 45 nt, Homo sapiens
ccugccuccccacugcucugccccacccuccaaucugcacuuuga

SEQ ID NO: 317 - RNA, 45 nt, Pan troglodytes; identical to SEQ ID NO: 316

SEQ ID NO: 318 - RNA, 47 nt, Mus sp.
uggagugcuccucugcuguccuccccacccuccagucugcacuuuga

SEQ ID NO: 319 - RNA, 44 nt, Rattus sp.
uggagcgcuccacugcccuucccacuggccagucugcacuuugg

SEQ ID NO: 320 - RNA, 45 nt, Canis familiaris
cugccccacucguugcucugccccacccuccaaucugcacuuuga

SEQ ID NO: 321 - RNA, 23 nt, Artificial Sequence
  Description of Artificial Sequence Synthetic oligonucleotide
agaaguaaacagauggcacuuug

SEQ ID NO: 322 - RNA, 24 nt, Artificial Sequence
  Description of Artificial Sequence Synthetic oligonucleotide
cccuccccaguucauugcacuuug

SEQ ID NO: 323 - RNA, 23 nt, Artificial Sequence
  Description of Artificial Sequence Synthetic oligonucleotide
agaaguaaacagaugggaguaug

SEQ ID NO: 324 - RNA, 24 nt, Artificial Sequence
  Description of Artificial Sequence Synthetic oligonucleotide
cccuccccaguucauuggaguaug